SEQUENCE LISTING (115 sequences)

SEQ ID NO: 1 - protein, 707 aa, Artificial Sequence
  Synthetic Construct
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 2 - protein, 757 aa, Artificial Sequence
  Synthetic Construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 3 - protein, 766 aa, Artificial Sequence
  Synthetic Construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 4 - protein, 765 aa, Artificial Sequence
  Synthetic Construct
MYSLEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 5 - protein, 793 aa, Artificial Sequence
  Synthetic Construct
MSSLPTPLELLKQKHADLFKGLQFSSKDNKMAGKVLKKDGEEAALAFLSERGVSRGELPNFRPPAKTLVVAQSRPFEEFPIYRVSEAIQLYVYSLSVKELETVPSGSSTKKEHQRFFQDSSVPDFGYTSVQGLNKIFGLARGIYLGVITRGENQLQKAKSKHEALNKKRRASGEAETEFDPTPYEYMTPERKLAKPPGVNHSIMCYVDISVDEFDFRNPDGIVLPSEYAGYCREINTAIEKGTVDRLGHLKGGPGYIPGHQRKESTTEGPKINFRKGRIRRSYTALYAKRDSRRVRQGKLALPSYRHHMMRLNSNAESAILAVIFFGKDWVVFDLRGLLRNVRWRNLFVDGSTPSTLLGMFGDPVIDPKRGVVAFCYKEQIVPVVSKSITKMVKAPELLNKLYLKSEDPLVLVAIDLGQTNPVGVGVYRVMNASLDYEVVTRFALESELLREIESYRQRTNAFEAQIRAETFDAMTSEEQEEITRVRAFSASKAKENVCHRFGMPVDAVDWATMGSNTIHIAKWVMRHGDPSLVEVLEYRKDNEIKLDKNGVPKKVKLTDKRIANLTSIRLRFSQETSKHYNDTMWELRRKHPVYQKLSKSKADFSRRVVNSIIRRVNHLVPRARIVFIIEDLKNLGKVFHGSGKRELGWDSYFEPKSENRWFIQVLHKAFSETGKHKGYYIIECWPNWTSCTCPKCSCCDSENRHGEVFRCLACGYTCNTDFGTAPDNLVKIATTGKGLPGPKKRCKGSSKGKNPKIARSSETGVSVTESGAPKVKKSSPTQTSQSSSQSAP

SEQ ID NO: 6 - protein, 441 aa, Artificial Sequence
  Synthetic Construct
MNKIEKEKTPLAKLMNENFAGLRFPFAIIKQAGKKLLKEGELKTIEYMTGKGSIEPLPNFKPPVKCLIVAKRRDLKYFPICKASCEIQSYVYSLNYKDFMDYFSTPMTSQKQHEEFFKKSGLNIEYQNVAGLNLIFNNVKNTYNGVILKVKNRNEKLKKKAIKNNYEFEEIKTFNDDGCLINKPGINNVIYCFQSISPKILKNITHLPKEYNDYDCSVDRNIIQKYVSRLDIPESQPGHVPEWQRKLPEFNNTNNPRRRRKWYSNGRNISKGYSVDQVNQAKIEDSLLAQIKIGEDWIILDIRGLLRDLNRRELISYKNKLTIKDVLGFFSDYPIIDIKKNLVTFCYKEGVIQVVSQKSIGNKKSKQLLEKLIENKPIALVSIDLGQTNPVSVKISKLNKINNKISIESFTYRFLNEEILKEIEKYRKDYDKLELKLINEA

SEQ ID NO: 7 - protein, 772 aa, Artificial Sequence
  Synthetic Construct
MSNTAVSTREHMSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 8 - protein, 717 aa, Artificial Sequence
  Synthetic Construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 9 - protein, 793 aa, Artificial Sequence
  Synthetic Construct
MRSSREIGDKILMRQPAEKTAFQVFRQEVIGTQKLSGGDAKTAGRLYKQGKMEAAREWLLKGARDDVPPNFQPPAKCLVVAVSHPFEEWDISKTNHDVQAYIYAQPLQAEGHLNGLSEKWEDTSADQHKLWFEKTGVPDRGLPVQAINKIAKAAVNRAFGVVRKVENRNEKRRSRDNRIAEHNRENGLTEVVREAPEVATNADGFLLHPPGIDPSILSYASVSPVPYNSSKHSFVRLPEEYQAYNVEPDAPIPQFVVEDRFAIPPGQPGYVPEWQRLKCSTNKHRRMRQWSNQDYKPKAGRRAKPLEFQAHLTRERAKGALLVVMRIKEDWVVFDVRGLLRNVEWRKVLSEEAREKLTLKGLLDLFTGDPVIDTKRGIVTFLYKAEITKILSKRTVKTKNARDLLLRLTEPGEDGLRREVGLVAVDLGQTHPIAAAIYRIGRTSAGALESTVLHRQGLREDQKEKLKEYRKRHTALDSRLRKEAFETLSVEQQKEIVTVSGSGAQITKDKVCNYLGVDPSTLPWEKMGSYTHFISDDFLRRGGDPNIVHFDRQPKKGKVSKKSQRIKRSDSQWVGRMRPRLSQETAKARMEADWAAQNENEEYKRLARSKQELARWCVNTLLQNTRCITQCDEIVVVIEDLNVKSLHGKGAREPGWDNFFTPKTENRWFIQILHKTFSELPKHRGEHVIEGCPLRTSITCPACSYCDKNSRNGEKFVCVACGATFHADFEVATYNLVRLATTGMPMPKSLERQGGGEKAGGARKARKKAKQVEKIVVQANANVTMNGASLHSP

SEQ ID NO: 10 - protein, 812 aa, Artificial Sequence
  Synthetic Construct
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 11 - DNA, 5131 bp, Arabidopsis thaliana
ctttggtgggcaaaaacatattagctgagaggtcaatttctttttcccctaaaccaaattacgttgagatgcatggtctctctctactcaattaaccaaataaggaaaagaatcatatggtcatcaattcgtaaatcaaaattttaatttgtgtggtatttaatccatctacatgtttcgtaagcaacaaaagagcttggtctgaaaaccaaacaagaccatatgggcactcgaatactccattttgttatcggctacttccactagcctcctccttcgctgcgtctcctgtttctctacttcacgattactcgctaggtaaaagataagatactaaacaactactatagcccctttaattcaagtccgtttttttttgttcttcttcttttcaattatgtgttaaagatacaaacttttgtctgatttgcttccaccggtttcacctaagatactcaattttcttactttttgtgtgttttgtaattctaattcttttatagcttcaatttttagattcattgaagcagttgtgagttaagttggagaaaatggttgtgtttgggaatgtttctgcggcgaatttgccttatcaaaacgggtttttggaggcactttcatctggaggttgtgaactaatgggacatagctttagggttcccacttctcaagcgcttaagacaagaacaaggaggaggagtactgctggtcctttgcaggtatctacctttctggtttttaacttggagttctcgaaatttcatctagtgtataagtgtaattttgattaaagagaggaaattgcaggtagtttgtgtggatattccaaggccagagctagagaacactgtcaatttcttggaagctgctagtttatctgcatccttccgtagtgctcctcgtcctgctaagcctttgaaagttgtaattgctggtgctggtatgtctacaacttcatttcttactgctttgtttgggatctgatggaattaatgttcatgtgatgaagttcttttggctctcaaataattgcaggattggctggattgtcaactgcaaagtacctggctgatgcaggccacaaacctctgttgcttgaagcaagagatgttcttggtggaaaggtactttgattggtctcactttccgaattacttattgttgtttgcatttctcattcttatatgatttgctgattcccaaaggctgtaccaattatgtttgtgagactacaaggtataactagtgtttagagtgttacagctcctaggtacccaatgtatctgtcctgtttgttgtattagtcttgtactgcccaatttgttttttattatgttctaatgtcttcagtcttgtgctacacaccgcacaaccctttccagtttctaggcttaatgtgaatcaaatatagagtagtttactggaagatatcttttggatatatattttgttaagttgttattgtgttctctggttcttaggttaggttcttgtttgttccgttttaacaatttacctatcttattggagagcgatatttatgatgtgatatttgtcttcctccagatagctgcatggaaggatgaagatggggactggtatgagactggtttacatattttctgtaagtccaaaactcataccctctctgttgctcccccaatatatttgttacatatatcttatcagctctaccatcaatttgaagaaagttgcaaaacactggttaaattatattgtatcttgttttccctgttattacttttcgttactgatactctcttcatgttcttgttggaagtcggtgcttatccgaatgtgcagaatttatttggagaacttgggatcaatgatcggttgcagtggaaggaacactccatgatttttgctatgccaagtaaacctggagaatttagtagatttgacttcccagatgtcctaccagcacccttaaatggtgagagaataaacttatcgcatattcttacttgctagtcagctgttataatattactgcttcaagtaattaaacttttaaactatctgatgaaaacagtggttgaactattagttagtggcgccatgtatttggtttgataaaattggattaatgtgcacacacttgttgaatgtaaaacaaagtatgatgtaattgagtaaaaaagtgaggttagtaactatgatgcatgtatttatttgtattgatcaattcaagctaattatagattttgacatttaatagtaatcaaagaactgatgaatgtttgaggatatgaagtatctccatctaatttagacgtcaggaagaacatggtcatttgtctttccaaagtaagaaattctagattttgagcttccccatactttaggatgggtaatcaaactgtaaaatattgttactggtcaaggcaagacgatataactgaactccgttgtagcattagcgctgaaatgttctgtggttgaattaatttgtttctggcaaataattaaagttgttgctgttggatttacgttgtaggtatttgggctattttgcggaacaacgagatgctgacatggccagagaaaataaagtttgctattggacttttgccagccatggtcggcggtcaggcttatgttgaggcccaagatggtttatcagtcaaagaatggatggaaaagcaggcaagtaggctattgagttcacagtttctgtgagattctcgtaagcatgttgatgagcttaacttggtagagtagttagcatgctgaatcatgtagtgttgatttgtcagctttcttatggatagctctgacttttctagtccatgtggttgttgtggtttcattgtcgtgcatgtgctttcactcagcagataataatacattctgattaattgtatacttgtccagggagtacctgagcgcgtgaccgacgaggtgtttattgccatgtcaaaggcgctaaactttataaaccctgatgaactgtcaatgcaatgcattttgatagctttgaaccggtttcttcaggtttgacactctagccctttatttctctttgttcttccttttgcctgtctttttgtgaatctttatctcactggcaatcatagttaccattcacatggtcgacatgaaaactataatagaatttttttacccaaacaatacttgagtttgatgttgattaacttgtactacctcatcccaataatcagatgtttctcattttgaaacttattgtgtgtcataagctgcaacagatggttaggatattgtgaatttgcaaatgcaaactaatatgaactacattgacaggaaaaacatggttccaagatggcattcttggatggtaatcctccggaaaggctttgtatgccagtagtggatcatattcgatcactaggtggggaagtgcaacttaattctaggataaagaaaattgagctcaatgacgatggcacggttaagagtttcttactcactaatggaagcactgtcgaaggagacgcttatgtgtttgccgctccaggtttactaccatgctgtcttgtgggtttaacaattcatctggtatcctgcttattaatatgttgtttatgtttttcttgtagtcgatatcctgaagctccttttaccagatccctggaaagaaataccgtacttcaagaaattggataaattagttggagtaccagttattaatgttcatatatggttagtgaaaccgttatctcccttgttttcttttggccattatgtgagaatcgagattacatgcaggtcttaactgctgtgaactaatattgcaggtttgatcgaaaactgaagaacacatatgatcacctactctttagcaggtgaatttcttatttatgcagttttgaggaattatgtcttatagaatacttagttggatacagtgttggaaaaaaagaatacaagtgttttgtcttgaatcagcaacagttaaggcttgttcttggttaaacatttgttgtatattcaacaatcttgttttgttgttttttgcagaagtaaccttctgagcgtgtatgccgacatgtccttaacttgtaaggtaagaacgcggcttgtttttctaaacgtgttcctccacttgtttatctatcataaattggtgctctgtggatcttctcataatttttgatggcttatcttgattccgaatctgattcaacaaagcgttttctttctatgataaactgtaggaatattacgatcctaaccggtcaatgctggagctagtatttgcaccagcagaggaatggatatcacggactgattctgacatcatagatgcaacaatgaaagaacttgagaaactcttccctgatgaaatctcagctgaccaaagcaaagctaaaattctgaagtaccatgtcgttaagactccaaggttagtgaagttgtaccatttatttagcttcaaatgagggaccaggctttacattgataaattcaacatctttctctattgttgcatgatcagatctgtgtacaagaccatcccaaactgtgaaccatgtcgtcctctacaaagatcacctattgaaggattctacttagctggagattacacaaaacagaagtacttagcttccatggaaggcgctgtcctctctggcaaattctgctctcagtctattgttcaggtaaacacaagcttgccattcaaatagttaaccagtaaccttgttctctgtttcttgttggaatatatcgccaataaccggtcggtttacctgattttgtctcactattatactgttccggttatgtagagaaattgctaaagtcagaatttactggttatacaggattacgagctactggctgcgtctggaccaagaaagttgtcggaggcaacagtatcatcatcatgagaagaggacaaaacttaaagatgatttgcttgtaagcattattatttgtgtataaatctcattgcaatccaaacttaaccttactctcttcagtaaatgaatctcacagatttgacatctcacgtttctgtcaattttataatttttaaaaagtaattactgtcgaccttttgtaatcatagtgatttatcattatgtctctctttttaaaaccttttctggtacaaattataaaa

SEQ ID NO: 12 - DNA, 1701 bp, Arabidopsis thaliana
atggttgtgtttgggaatgtttctgcggcgaatttgccttatcaaaacgggtttttggaggcactttcatctggaggttgtgaactaatgggacatagctttagggttcccacttctcaagcgcttaagacaagaacaaggaggaggagtactgctggtcctttgcaggtagtttgtgtggatattccaaggccagagctagagaacactgtcaatttcttggaagctgctagtttatctgcatccttccgtagtgctcctcgtcctgctaagcctttgaaagttgtaattgctggtgctggattggctggattgtcaactgcaaagtacctggctgatgcaggccacaaacctctgttgcttgaagcaagagatgttcttggtggaaagatagctgcatggaaggatgaagatggggactggtatgagactggtttacatattttcttcggtgcttatccgaatgtgcagaatttatttggagaacttgggatcaatgatcggttgcagtggaaggaacactccatgatttttgctatgccaagtaaacctggagaatttagtagatttgacttcccagatgtcctaccagcacccttaaatggtatttgggctattttgcggaacaacgagatgctgacatggccagagaaaataaagtttgctattggacttttgccagccatggtcggcggtcaggcttatgttgaggcccaagatggtttatcagtcaaagaatggatggaaaagcagggagtacctgagcgcgtgaccgacgaggtgtttattgccatgtcaaaggcgctaaactttataaaccctgatgaactgtcaatgcaatgcattttgatagctttgaaccggtttcttcaggaaaaacatggttccaagatggcattcttggatggtaatcctccggaaaggctttgtatgccagtagtggatcatattcgatcactaggtggggaagtgcaacttaattctaggataaagaaaattgagctcaatgacgatggcacggttaagagtttcttactcactaatggaagcactgtcgaaggagacgcttatgtgtttgccgctccagtcgatatcctgaagctccttttaccagatccctggaaagaaataccgtacttcaagaaattggataaattagttggagtaccagttattaatgttcatatatggtttgatcgaaaactgaagaacacatatgatcacctactctttagcagaagtaaccttctgagcgtgtatgccgacatgtccttaacttgtaaggaatattacgatcctaaccggtcaatgctggagctagtatttgcaccagcagaggaatggatatcacggactgattctgacatcatagatgcaacaatgaaagaacttgagaaactcttccctgatgaaatctcagctgaccaaagcaaagctaaaattctgaagtaccatgtcgttaagactccaagatctgtgtacaagaccatcccaaactgtgaaccatgtcgtcctctacaaagatcacctattgaaggattctacttagctggagattacacaaaacagaagtacttagcttccatggaaggcgctgtcctctctggcaaattctgctctcagtctattgttcaggattacgagctactggctgcgtctggaccaagaaagttgtcggaggcaacagtatcatcatcatga

SEQ ID NO: 13 - DNA, 14796 bp, Artificial Sequence
  Synthetic Construct
gtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagatcactagtgatcagatgcagagagactaacctcagataggcgacttcaaaaccccccgaaaatttgacaatggagcttgaaaagaatggcaaaaaaaagtgcctccaaaaacccgtttgtcccctcgtgaggggcaatcgttgagcgttccgaccaatcactacttcgactctagctgtatataaactcagcttcgttttcttatctaagcgatgtgggacttttgaagattgttttcaacttaaatgggcctatataagaaatactattgttctttcccatataaatgggcctgcttctcttctttcagattcccaggggccttttgaagattatcttcatatcttaagaatgaagatgttttattcaatcaaattcttgaaggttcgatgcctaatcattctaatcctgggacaaactatgaaacaagatacaaaaactccgaatggaaagttaaaaagaagaaaacgaaagctacggttcaagaaaatgtaagctgataaacaaaaaaaaactgtatgaacgaagaagaagaaaaaaagctaagaagaaatgatgtattgtgcggaaggcaagtcgagtttccgttgttcaacgaagcttctagtatcctaggaaggtaccagtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatggattacaaggatgatgatgataaggattacaaggatgatgatgataagatggctccaaagaagaagagaaaggttggaatccacggagttccagctgctatgccgaagcccgccgtcgaatcagagttttccaaagtcctcaagaaacactttcctggggagcgttttaggtctagctatatgaagagggggggtaaaattctggcagcacaaggcgaggaagctgtagtggcgtacttgcagggaaagagtgaggaggaaccgccgaattttcagccgccggcgaagtgccacgtggtcaccaaaagcagggatttcgcagaatggcccataatgaaagcctctgaagccatacagaggtacatctatgcgctcagcactacagagcgagctgcctgcaaaccgggtaagagctcagaaagtcacgcggcctggttcgcggctacaggggtgagcaatcacggctattctcatgtacaaggtcttaacctgatctttgaccacacgctaggacgatacgatggcgttttaaagaaagtacagcttcgaaatgagaaggcccgagctagattggaaagcataaacgcctcacgagctgatgaaggattgccggaaatcaaggccgaggaggaggaggtagctacaaatgaaacaggtcatctactacagccgccaggcataaacccatcattctacgtctaccagaccatatctccgcaggcttaccgaccaagggacgaaatagtgttaccacccgagtacgccggttacgtcagggatccgaacgctccgattccactgggcgtggtcaggaaccgttgtgacatacagaagggttgcccgggatatatacccgagtggcagagggaagctggtacggcaattagtcccaagacaggaaaagcagtgacggttccaggacttagcccgaagaagaataaacgtatgcgtaggtactggaggtcagaaaaggagaaggctcaagatgcacttctcgtaactgtaaggataggtaccgattgggtagtcatagacgtaagaggattactaagaaacgccaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggaggactattgctcccaaggacataagtttaaatgcacttctagatttatttaccggtgatccagtcatcgatgtcagacgaaacatcgtgaccttcacctataccttggacgcttgcggaacttatgctagaaaatggactctcaagggaaaacagacaaaagcaaccttagataaactgacagcgacacaaactgtggccttagttgctatagatctgggacaaacaaacccaattagcgcgggtatcagtcgtgtcacacaggagaacggggccctccagtgcgaaccgcttgatcgttttacattgcctgatgaccttttgaaagatatttctgcgtaccgaattgcatgggaccgtaacgaggaggaactcagggccagatccgttgaggcactcccagaggcacaacaagcagaggtcagagctctggacggggtctccaaagagaccgcgcgtacacagttgtgcgcggacttcggtctggacccaaagcgactaccgtgggataaaatgagtagcaataccacgttcataagcgaggcgctcctttccaacagcgtatcccgtgaccaagtattctttaccccggccccaaagaaaggagccaagaagaaagcaccggtggaagtgatgcgaaaagacaggacatgggcgcgagcgtacaaaccacgactctcagtagaagcacaaaagttgaaaaatgaggctctttgggctttgaagcgtacctctccagaatatctaaagttgtcacgacgtaaagaagaattgtgtaggaggtccattaattacgtgatagaaaaaactagaaggcgaacccaatgccaaattgtgatccccgttatagaagatctaaatgtccgatttttccacgggtctggcaaacgactcccgggctgggataacttcttcacggcaaagaaggaaaaccgatggtttatccaagggctacataaggcgttttctgacttaaggacccaccgatccttctacgtgttcgaggtgcgtccagagagaacatcaatcacctgtccgaaatgcgggcactgtgaagtggggaacagggatggagaagcctttcagtgtctcagttgcggtaagacatgcaacgcagatctggacgtagcgacacataacttgactcaagtggcgctcaccggcaaaacaatgccgaagagagaggagcctagagatgcacaagggacagcccccgcgagaaagacgaagaaagctagcaagtcaaaggcacccccggctgaacgtgaggatcagactcccgctcaagagccgtcccaaacgtccaagagaccagctgctaccaagaaggctggacaggctaagaagaagaagtgaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacgaattc

SEQ ID NO: 14 - DNA, 14769 bp, Artificial Sequence
  Synthetic Construct
gtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagatcactagtgatcagatgcagagagactaacctcagataggcgacttcaaaaccccccgaaaatttgacaatggagcttgaaaagaatggcaaaaaaaagtgcctccaaaaacccgtttgtcccctcgtgaggggcaatcgttgagcgttccgaccaatcactacttcgactctagctgtatataaactcagcttcgttttcttatctaagcgatgtgggacttttgaagattgttttcaacttaaatgggcctatataagaaatactattgttctttcccatataaatgggcctgcttctcttctttcagattcccaggggccttttgaagattatcttcatatcttaagaatgaagatgttttattcaatcaaattcttgaaggttcgatgcctaatcattctaatcctgggacaaactatgaaacaagatacaaaaactccgaatggaaagttaaaaagaagaaaacgaaagctacggttcaagaaaatgtaagctgataaacaaaaaaaaactgtatgaacgaagaagaagaaaaaaagctaagaagaaatgatgtattgtgcggaaggcaagtcgagtttccgttgttcaacgaagcttctagtatcctaggaaggtaccagtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgccgaagcccgccgtcgaatcagagttttccaaagtcctcaagaaacactttcctggggagcgttttaggtctagctatatgaagagggggggtaaaattctggcagcacaaggcgaggaagctgtagtggcgtacttgcagggaaagagtgaggaggaaccgccgaattttcagccgccggcgaagtgccacgtggtcaccaaaagcagggatttcgcagaatggcccataatgaaagcctctgaagccatacagaggtacatctatgcgctcagcactacagagcgagctgcctgcaaaccgggtaagagctcagaaagtcacgcggcctggttcgcggctacaggggtgagcaatcacggctattctcatgtacaaggtcttaacctgatctttgaccacacgctaggacgatacgatggcgttttaaagaaagtacagcttcgaaatgagaaggcccgagctagattggaaagcataaacgcctcacgagctgatgaaggattgccggaaatcaaggccgaggaggaggaggtagctacaaatgaaacaggtcatctactacagccgccaggcataaacccatcattctacgtctaccagaccatatctccgcaggcttaccgaccaagggacgaaatagtgttaccacccgagtacgccggttacgtcagggatccgaacgctccgattccactgggcgtggtcaggaaccgttgtgacatacagaagggttgcccgggatatatacccgagtggcagagggaagctggtacggcaattagtcccaagacaggaaaagcagtgacggttccaggacttagcccgaagaagaataaacgtatgcgtaggtactggaggtcagaaaaggagaaggctcaagatgcacttctcgtaactgtaaggataggtaccgattgggtagtcatagacgtaagaggattactaagaaacgccaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggaggactattgctcccaaggacataagtttaaatgcacttctagatttatttaccggtgatccagtcatcgatgtcagacgaaacatcgtgaccttcacctataccttggacgcttgcggaacttatgctagaaaatggactctcaagggaaaacagacaaaagcaaccttagataaactgacagcgacacaaactgtggccttagttgctatagatctgggacaaacaaacccaattagcgcgggtatcagtcgtgtcacacaggagaacggggccctccagtgcgaaccgcttgatcgttttacattgcctgatgaccttttgaaagatatttctgcgtaccgaattgcatgggaccgtaacgaggaggaactcagggccagatccgttgaggcactcccagaggcacaacaagcagaggtcagagctctggacggggtctccaaagagaccgcgcgtacacagttgtgcgcggacttcggtctggacccaaagcgactaccgtgggataaaatgagtagcaataccacgttcataagcgaggcgctcctttccaacagcgtatcccgtgaccaagtattctttaccccggccccaaagaaaggagccaagaagaaagcaccggtggaagtgatgcgaaaagacaggacatgggcgcgagcgtacaaaccacgactctcagtagaagcacaaaagttgaaaaatgaggctctttgggctttgaagcgtacctctccagaatatctaaagttgtcacgacgtaaagaagaattgtgtaggaggtccattaattacgtgatagaaaaaactagaaggcgaacccaatgccaaattgtgatccccgttatagaagatctaaatgtccgatttttccacgggtctggcaaacgactcccgggctgggataacttcttcacggcaaagaaggaaaaccgatggtttatccaagggctacataaggcgttttctgacttaaggacccaccgatccttctacgtgttcgaggtgcgtccagagagaacatcaatcacctgtccgaaatgcgggcactgtgaagtggggaacagggatggagaagcctttcagtgtctcagttgcggtaagacatgcaacgcagatctggacgtagcgacacataacttgactcaagtggcgctcaccggcaaaacaatgccgaagagagaggagcctagagatgcacaagggacagcccccgcgagaaagacgaagaaagctagcaagtcaaaggcacccccggctgaacgtgaggatcagactcccgctcaagagccgtcccaaacgtccggatccggaccgaagaaaaagcgaaaggtagaggatcctaaaaagaagcgtaaagtctccttgggttctggctccgactataaggatgacgatgacaaagactataaggatgacgacgacaagtgaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacgaattc

SEQ ID NO: 15 - DNA, 189 bp, Artificial Sequence
  Synthetic Construct
gtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcag

SEQ ID NO: 16 - DNA, 36 bp, Artificial Sequence
  Synthetic Construct
gtcccctcgtgaggggcaatcgttgagcgttccgac

SEQ ID NO: 17 - protein, 807 aa, Artificial Sequence
  Synthetic Construct
MDYKDDDDKDYKDDDDKMAPKKKRKVGIHGVPAAMPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTSKRPAATKKAGQAKKKK

SEQ ID NO: 18 - protein, 8 aa, Artificial Sequence
  Synthetic Construct
DYKDDDDK

SEQ ID NO: 19 - protein, 7 aa, Artificial Sequence
  Synthetic Construct
PKKKRKV

SEQ ID NO: 20 - protein, 16 aa, Artificial Sequence
  Synthetic Construct
KRPAATKKAGQAKKKK

SEQ ID NO: 21 - protein, 798 aa, Artificial Sequence
  Synthetic Construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTSGSGPKKKRKVEDPKKKRKVSLGSGSDYKDDDDKDYKDDDDK

SEQ ID NO: 22 - DNA, 1018 bp, Artificial Sequence
  Synthetic Construct
gatgggaaattcattgaaaaccctaaacccaaatcaacagctgcaattcaaaaggggactaattgacaaacaaaaattgataacaaatagaggtagggggagagtttcgtacgcgacaatgagattgagctcttgaggacttgtgaagttgccaacgcacgagtgagtgacactggtcggtttgtgagccgtaacaacgtagttccatgagctcatcttcctcttctttgtctccagggaatttgagttcgactttctacgcgagggccctcgaggaagcttctagatttctgaatcgagctttcggaattttaacatagagaagttagagagagaatgaaaagccaaaggaggcgaaaatcgaacaaggaagaagaaagacaactttcgacaaagactggtcggtcggttttggtagacaattgaaattagatggatggtccggttcggtatactataagattaaaaacagttttaaattcagctaaaccgaactcatttgattttattaaaccggaatcatccgattcgagtttgtaaaaaataccgaaattgaaaacactaaacaaaaactgtattaaactgttactgaaataagagaatctcccaattcggtttacgtactactcttcagaaatcagaaccaaaaattcagaaatcggattgaaccaaacttaaattgacggtccggttagtcttcggctctacaaattaaaggcccaagtttctgctttaaaagaacgaaatagttaatgggctcaaaccatagaccaggtaagtcatgggcttggttagtccgggtcaacccggtagacccgattcctgaagaaaacctagtggaaggtttaaagttgtaaactttccgaccaaataaacaaaatcgttttccagcttcttccgtcgccactaaaccctgaggctaaacctagacgagtcaaagtgtaaaatcgttaaaccctaagagggagtgagagagagaagaatgaagtacaacaacgagaagaagaaaggagtcgag

SEQ ID NO: 23 - DNA, 1994 bp, Artificial Sequence
  Synthetic Construct
atgcatatgagtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaaca

SEQ ID NO: 24 - DNA, 449 bp, Artificial Sequence
  Synthetic Construct
aagctttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgatt

SEQ ID NO: 25 - DNA, 13498 bp, Artificial Sequence
  Synthetic Construct
gtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagatcactagtgatcagatgcagagagactaacctcagataggcgacttcaaaaccccccgaaaatttgacaatggagcttgaaaagaatggcaaaaaaaaattggctggattgtcaactggtcccctcgtgaggggcaatcgttgagcgttccgaccaatcactacttcgactctagctgtatataaactcagcttcgttttcttatctaagcgatgtgggacttttgaagattgttttcaacttaaatgggcctatataagaaatactattgttctttcccatataaatgggcctgcttctcttctttcagattcccaggggccttttgaagattatcttcatatcttaagaatgaagatgttttattcaatcaaattcttgaaggttcgatgcctaatcattctaatcctgggacaaactatgaaacaagatacaaaaactccgaatggaaagttaaaaagaagaaaacgaaagctacggttcaagaaaatgtaagctgataaacaaaaaaaaactgtatgaacgaagaagaagaaaaaaagctaagaagaaatgatgtattgtgcggaaggcaagtcgagtttccgttgttcaacgaagcttctagtatcctaggaaggtaccgggccccccctcgacgatgggaaattcattgaaaaccctaaacccaaatcaacagctgcaattcaaaaggggactaattgacaaacaaaaattgataacaaatagaggtagggggagagtttcgtacgcgacaatgagattgagctcttgaggacttgtgaagttgccaacgcacgagtgagtgacactggtcggtttgtgagccgtaacaacgtagttccatgagctcatcttcctcttctttgtctccagggaatttgagttcgactttctacgcgagggccctcgaggaagcttctagatttctgaatcgagctttcggaattttaacatagagaagttagagagagaatgaaaagccaaaggaggcgaaaatcgaacaaggaagaagaaagacaactttcgacaaagactggtcggtcggttttggtagacaattgaaattagatggatggtccggttcggtatactataagattaaaaacagttttaaattcagctaaaccgaactcatttgattttattaaaccggaatcatccgattcgagtttgtaaaaaataccgaaattgaaaacactaaacaaaaactgtattaaactgttactgaaataagagaatctcccaattcggtttacgtactactcttcagaaatcagaaccaaaaattcagaaatcggattgaaccaaacttaaattgacggtccggttagtcttcggctctacaaattaaaggcccaagtttctgctttaaaagaacgaaatagttaatgggctcaaaccatagaccaggtaagtcatgggcttggttagtccgggtcaacccggtagacccgattcctgaagaaaacctagtggaaggtttaaagttgtaaactttccgaccaaataaacaaaatcgttttccagcttcttccgtcgccactaaaccctgaggctaaacctagacgagtcaaagtgtaaaatcgttaaaccctaagagggagtgagagagagaagaatgaagtacaacaacgagaagaagaaaggagtcgagatggattacaaggatgatgatgataaggattacaaggatgatgatgataagatggctccaaagaagaagagaaaggttggaatccacggagttccagctgctatgccgaagcccgccgtcgaatcagagttttccaaagtcctcaagaaacactttcctggggagcgttttaggtctagctatatgaagagggggggtaaaattctggcagcacaaggcgaggaagctgtagtggcgtacttgcagggaaagagtgaggaggaaccgccgaattttcagccgccggcgaagtgccacgtggtcaccaaaagcagggatttcgcagaatggcccataatgaaagcctctgaagccatacagaggtacatctatgcgctcagcactacagagcgagctgcctgcaaaccgggtaagagctcagaaagtcacgcggcctggttcgcggctacaggggtgagcaatcacggctattctcatgtacaaggtcttaacctgatctttgaccacacgctaggacgatacgatggcgttttaaagaaagtacagcttcgaaatgagaaggcccgagctagattggaaagcataaacgcctcacgagctgatgaaggattgccggaaatcaaggccgaggaggaggaggtagctacaaatgaaacaggtcatctactacagccgccaggcataaacccatcattctacgtctaccagaccatatctccgcaggcttaccgaccaagggacgaaatagtgttaccacccgagtacgccggttacgtcagggatccgaacgctccgattccactgggcgtggtcaggaaccgttgtgacatacagaagggttgcccgggatatatacccgagtggcagagggaagctggtacggcaattagtcccaagacaggaaaagcagtgacggttccaggacttagcccgaagaagaataaacgtatgcgtaggtactggaggtcagaaaaggagaaggctcaagatgcacttctcgtaactgtaaggataggtaccgattgggtagtcatagacgtaagaggattactaagaaacgccaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggaggactattgctcccaaggacataagtttaaatgcacttctagatttatttaccggtgatccagtcatcgatgtcagacgaaacatcgtgaccttcacctataccttggacgcttgcggaacttatgctagaaaatggactctcaagggaaaacagacaaaagcaaccttagataaactgacagcgacacaaactgtggccttagttgctatagatctgggacaaacaaacccaattagcgcgggtatcagtcgtgtcacacaggagaacggggccctccagtgcgaaccgcttgatcgttttacattgcctgatgaccttttgaaagatatttctgcgtaccgaattgcatgggaccgtaacgaggaggaactcagggccagatccgttgaggcactcccagaggcacaacaagcagaggtcagagctctggacggggtctccaaagagaccgcgcgtacacagttgtgcgcggacttcggtctggacccaaagcgactaccgtgggataaaatgagtagcaataccacgttcataagcgaggcgctcctttccaacagcgtatcccgtgaccaagtattctttaccccggccccaaagaaaggagccaagaagaaagcaccggtggaagtgatgcgaaaagacaggacatgggcgcgagcgtacaaaccacgactctcagtagaagcacaaaagttgaaaaatgaggctctttgggctttgaagcgtacctctccagaatatctaaagttgtcacgacgtaaagaagaattgtgtaggaggtccattaattacgtgatagaaaaaactagaaggcgaacccaatgccaaattgtgatccccgttatagaagatctaaatgtccgatttttccacgggtctggcaaacgactcccgggctgggataacttcttcacggcaaagaaggaaaaccgatggtttatccaagggctacataaggcgttttctgacttaaggacccaccgatccttctacgtgttcgaggtgcgtccagagagaacatcaatcacctgtccgaaatgcgggcactgtgaagtggggaacagggatggagaagcctttcagtgtctcagttgcggtaagacatgcaacgcagatctggacgtagcgacacataacttgactcaagtggcgctcaccggcaaaacaatgccgaagagagaggagcctagagatgcacaagggacagcccccgcgagaaagacgaagaaagctagcaagtcaaaggcacccccggctgaacgtgaggatcagactcccgctcaagagccgtcccaaacgtccaagagaccagctgctaccaagaaggctggacaggctaagaagaagaagtgaggatcctgattgatcgatagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattc

SEQ ID NO: 26 - DNA, 13471 bp, Artificial Sequence
  Synthetic Construct
gtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagatcactagtgatcagatgcagagagactaacctcagataggcgacttcaaaaccccccgaaaatttgacaatggagcttgaaaagaatggcaaaaaaaaattggctggattgtcaactggtcccctcgtgaggggcaatcgttgagcgttccgaccaatcactacttcgactctagctgtatataaactcagcttcgttttcttatctaagcgatgtgggacttttgaagattgttttcaacttaaatgggcctatataagaaatactattgttctttcccatataaatgggcctgcttctcttctttcagattcccaggggccttttgaagattatcttcatatcttaagaatgaagatgttttattcaatcaaattcttgaaggttcgatgcctaatcattctaatcctgggacaaactatgaaacaagatacaaaaactccgaatggaaagttaaaaagaagaaaacgaaagctacggttcaagaaaatgtaagctgataaacaaaaaaaaactgtatgaacgaagaagaagaaaaaaagctaagaagaaatgatgtattgtgcggaaggcaagtcgagtttccgttgttcaacgaagcttctagtatcctaggaaggtaccgggccccccctcgacgatgggaaattcattgaaaaccctaaacccaaatcaacagctgcaattcaaaaggggactaattgacaaacaaaaattgataacaaatagaggtagggggagagtttcgtacgcgacaatgagattgagctcttgaggacttgtgaagttgccaacgcacgagtgagtgacactggtcggtttgtgagccgtaacaacgtagttccatgagctcatcttcctcttctttgtctccagggaatttgagttcgactttctacgcgagggccctcgaggaagcttctagatttctgaatcgagctttcggaattttaacatagagaagttagagagagaatgaaaagccaaaggaggcgaaaatcgaacaaggaagaagaaagacaactttcgacaaagactggtcggtcggttttggtagacaattgaaattagatggatggtccggttcggtatactataagattaaaaacagttttaaattcagctaaaccgaactcatttgattttattaaaccggaatcatccgattcgagtttgtaaaaaataccgaaattgaaaacactaaacaaaaactgtattaaactgttactgaaataagagaatctcccaattcggtttacgtactactcttcagaaatcagaaccaaaaattcagaaatcggattgaaccaaacttaaattgacggtccggttagtcttcggctctacaaattaaaggcccaagtttctgctttaaaagaacgaaatagttaatgggctcaaaccatagaccaggtaagtcatgggcttggttagtccgggtcaacccggtagacccgattcctgaagaaaacctagtggaaggtttaaagttgtaaactttccgaccaaataaacaaaatcgttttccagcttcttccgtcgccactaaaccctgaggctaaacctagacgagtcaaagtgtaaaatcgttaaaccctaagagggagtgagagagagaagaatgaagtacaacaacgagaagaagaaaggagtcgagatgccgaagcccgccgtcgaatcagagttttccaaagtcctcaagaaacactttcctggggagcgttttaggtctagctatatgaagagggggggtaaaattctggcagcacaaggcgaggaagctgtagtggcgtacttgcagggaaagagtgaggaggaaccgccgaattttcagccgccggcgaagtgccacgtggtcaccaaaagcagggatttcgcagaatggcccataatgaaagcctctgaagccatacagaggtacatctatgcgctcagcactacagagcgagctgcctgcaaaccgggtaagagctcagaaagtcacgcggcctggttcgcggctacaggggtgagcaatcacggctattctcatgtacaaggtcttaacctgatctttgaccacacgctaggacgatacgatggcgttttaaagaaagtacagcttcgaaatgagaaggcccgagctagattggaaagcataaacgcctcacgagctgatgaaggattgccggaaatcaaggccgaggaggaggaggtagctacaaatgaaacaggtcatctactacagccgccaggcataaacccatcattctacgtctaccagaccatatctccgcaggcttaccgaccaagggacgaaatagtgttaccacccgagtacgccggttacgtcagggatccgaacgctccgattccactgggcgtggtcaggaaccgttgtgacatacagaagggttgcccgggatatatacccgagtggcagagggaagctggtacggcaattagtcccaagacaggaaaagcagtgacggttccaggacttagcccgaagaagaataaacgtatgcgtaggtactggaggtcagaaaaggagaaggctcaagatgcacttctcgtaactgtaaggataggtaccgattgggtagtcatagacgtaagaggattactaagaaacgccaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggaggactattgctcccaaggacataagtttaaatgcacttctagatttatttaccggtgatccagtcatcgatgtcagacgaaacatcgtgaccttcacctataccttggacgcttgcggaacttatgctagaaaatggactctcaagggaaaacagacaaaagcaaccttagataaactgacagcgacacaaactgtggccttagttgctatagatctgggacaaacaaacccaattagcgcgggtatcagtcgtgtcacacaggagaacggggccctccagtgcgaaccgcttgatcgttttacattgcctgatgaccttttgaaagatatttctgcgtaccgaattgcatgggaccgtaacgaggaggaactcagggccagatccgttgaggcactcccagaggcacaacaagcagaggtcagagctctggacggggtctccaaagagaccgcgcgtacacagttgtgcgcggacttcggtctggacccaaagcgactaccgtgggataaaatgagtagcaataccacgttcataagcgaggcgctcctttccaacagcgtatcccgtgaccaagtattctttaccccggccccaaagaaaggagccaagaagaaagcaccggtggaagtgatgcgaaaagacaggacatgggcgcgagcgtacaaaccacgactctcagtagaagcacaaaagttgaaaaatgaggctctttgggctttgaagcgtacctctccagaatatctaaagttgtcacgacgtaaagaagaattgtgtaggaggtccattaattacgtgatagaaaaaactagaaggcgaacccaatgccaaattgtgatccccgttatagaagatctaaatgtccgatttttccacgggtctggcaaacgactcccgggctgggataacttcttcacggcaaagaaggaaaaccgatggtttatccaagggctacataaggcgttttctgacttaaggacccaccgatccttctacgtgttcgaggtgcgtccagagagaacatcaatcacctgtccgaaatgcgggcactgtgaagtggggaacagggatggagaagcctttcagtgtctcagttgcggtaagacatgcaacgcagatctggacgtagcgacacataacttgactcaagtggcgctcaccggcaaaacaatgccgaagagagaggagcctagagatgcacaagggacagcccccgcgagaaagacgaagaaagctagcaagtcaaaggcacccccggctgaacgtgaggatcagactcccgctcaagagccgtcccaaacgtccggatccggaccgaagaaaaagcgaaaggtagaggatcctaaaaagaagcgtaaagtctccttgggttctggctccgactataaggatgacgatgacaaagactataaggatgacgacgacaagtgaggatcctgattgatcgatagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattc

SEQ ID NO: 27 - DNA, 5105 bp, Artificial Sequence
  Synthetic Construct
aatgagtatccctatatatatatatatatattaaacttcttttttcggtcaatacaattttataatctttcattttttctatcatttcatatcattgtaactataaattttcgtaaatagacctttagtgttaatacaatagatttttattaattttatatcggattttgtttaaaaaagaaaaaccataggatggatgatgattggtacttataagattgtaattgggtatttttggattgttaccaccattacaaagctattaacagagattgaagatatcacacaatgagagcgccacagcttcagcaacgtcccatgcagctgatgtgccttcgcctttctcttcctcatctgcgcttataaataaggcaaagcaactagaaaagattaaaaccaaaaccaaaacaaaaaactagttaagaccctgattttgtttcataggtacatacacttttcaacattgatttttgttgttaaaaataaaatccatgtgaaggttctcatcatataccgaaagaatgggaaatttgaaaattccatacttttttaaaaagacaatttgttttatcactttagtttttttatatattcagcgtctaccaaatctacacttttttttctttctcgatttagttaatcttcgttcttgtgtcatgtaatagattactatttcaaaacatagatatttagttatctaaataaaactaggccatccatggatggtttcaattttttttttcatatgaaagaaaagttaaatttcatttcacaataaccattgattactaaatttagtaaagaatcaattgggtttagtgtttacttgtttaaggttttttttttttcttttgttatggttctatactaatatcaaagagttatgggccgaagcccatacatctttccgtcgagaatctcatatattctttatcgaagcccatacatctttccgtcgagaatctcatatataccttatcccattcaacattcatacgagcgccgctctagggtttttgcttttcgccattggtccaagtgctatttggttgtttaaggttgcttttagcacacaactttaatattatttttatgtttttcttcttacgatttatcgatttgtgggatactgacaatcagattattgttgttttttccagccaaatatcagatcttgcgccgctctttatcccattcaacattcatacgagcaccgctttacggtttttgcttttcgacattggtcgaagtgctatttggttgtttaaggttgcttttagcacacaactttaatattatttttatgttttcttcttacgatttatcgatttgtaggatactgacaatcagatttttgttgtttttttcagccaaaaatcagatttttttacattttgtttagagaatgattttggttcccgatttgtctgtttttcgcttatgtgtaaagtactttgaaaaatattgtgttaactctacaatgggtatcccaaatttttgagttcttttgtcttgttcgttgtcgagacactagaaatgttaatttaattctcttcttccaaaaagaaccatttactggttcattctctacttgaattttattctggttgtatttcttttccagtataaagcagattgttttttgttatttttcagtttagattggctttgtctcttttgagttgttgcaattgtcaaactttggaatgaaatagtaaataatcttaggttggtagtaaatcttaacattgtgtttttggggcataatttatcgataaaatcttcagcattaaaaccaaaaagaaaaaactttttaagtcttttttgtttggtggttaatataaagtttatacgtgtattaatttgatcacactcactatatgtccagggagctaaacctctatatcgagtactaatagtatgcaatgtccaggttattgcattgagggaaaatgaatggacaaggtgatttggatgcggttggaaacattccaaaaccaggtgaagctgaaggcgatgagattgatatgattaatgatatgtctggtgttaatgatcaagatggtggaaggatgagaagaacccataggcgcactgcttatcaaactcaagaacttgaaaagtttgttcactttcttcttcatttcatcatcatgcaacatttcctattattttttttatttttttattttgagtttggaatgtttctctttactttgctctttactttaaaatgagtgtagtttctacatggaaaatcctcatcccactgaagaacagaggtacgagcttggacaaaggcttaatatgggtgtcaatcaagtcaagaattggttccagaataaaagaaatctcgagaaggtaccaaaaaatacagaagtgtatacatgcatgtggctgtgtcttttcttattctttgcacgtaccgtattatggaccatgtttctgccctatgttaaaaacaaatactgtatatagtttaccacctctctctctcatcctcaaaatctgccttgattatggtataattattgagtgtggttttttgggtaagcagatcaataatgaccaccttgagaatgtaactcttagagaagagcatgacagattgctagcaactcaggatcagcttagaagcgcaatgctacgtagtttatgcaacatttgtggtaaggcaactaattgtggagacactgaatatgaggtgcaaaaacttatggctgagaatgctaacttggagcgggagatagaccagttcaattccagatacctttctcatcccaaacaaaggatggtcagtacatccgaacaggcgccttcttcttcctctaatccaggaataaatgcaacaccagtacttgattttagtggtggaactaggacgtctgagaaggagacatcaatttttctgaatcttgccattacggctttgagagagttgattacattgggagaagtggactgtccattttggatgatagatccaatcgttagatccaaaggagtatcaaagatctatgagaagtatagaagttctttcaacaatgtcacaaaacctcccgggcaaattgtggaggcttcaagagctaaaggtttagttcccatgacttgcgtgactctggtcaagactcttatggacacagtaataacctttaaaccaccacccattttttagtgaaatgttgttacatgaaatgttaacaagttttttttttttttttttaaacagggcaaatgggtcaacgtgtttgcacctatagtccctgtggcatcaacccataaagtgctatctaccggttctggtggaaccaaaagtggctcactccaacaggtataatatatatagttttctgagtcgtctatattggttgataatccaagagaagtagaaacatttcaaatttgtgatcatataattttggcagattcaagcagaatttcaagtaatttctccgctggtaccaaagagaaaagtaacgtttattagatactgcaaagagatcagacagggcttatgggtggtcgtcgacgttactcctactcaaaatccgactttgctgccctatggttgttctaagaggctaccctcaggccttatcatagacgacctgtccaatgggtactcccaggttacttctgaatcctgttctttgaacaatatatgttggttcacttgttattactcgcaatcataattaatgatctatcagagcatcttgtactgtggagacataaataaatggttaatggttattattatattcaccacatgtataacacatagagatttgatttcacaggttacatggattgaacaagcggaatataatgagagtcacatccaccaactctaccagcctttgattggctatgggatcgggctaggtgcaaagagatggctcgcgacgctgcagagacactgcgaaagcctctcgaccctttcatctaccaacttgactgaaattagtccaggtgaaagctatttaaattatagaataaatctcattacaactactttttaggtcattttcggttctccactaaatttggacaggccttgattgtgatatatgttttcgatataggattgtctgcaaaaggtgcaactgaaatagtgaagctagcacagcgaatgactctcaactactacagaggtattacgagtccttcggtggacaagtggcagaaaattcaggtggagaatgtggcacaaaacatgagtttcatgatccgaaagaacgtgaatgagcctggtgagctaactgggattgtgctgagtgcatccacttctgtttggctcccagtgaaccagcatacactctttgctttcattagccacctgagtttcagacacgagtgggatatcttgaccaatgatactaccatggaagaaacaatccggattcaaaaagcaaaacgccatggaaacatcatctctcttctgaaaatcgttgtgagtttcaaaacatttatttttggtttaatgagtgtcgttttgtctatgtttccattgattttcgtgagttttgcaatgcaaacagaataatggtatgctggttctgcaagagatttggaatgatgcatcaggtgcaatggtggtgtatgcaccagtggaaaccaattctattgagctggtcaagagaggtgaaaattcagattctgtgaagtttcttccttcgggattttcgatagtgccagatggagtaaatgggtcatatcatagaggcaatactggtggaggatgtctactgacatttggacttcagatcttggtgggcatcaatccaactgctgcactcattcaaggtactgtcaaaagtgtcgagacactcatggctcatactattgtcaagatcaaatccgcgttagatttacagacgtaaccatcagcagctccagtttccgttgttccgcaagaaattcgacattggcttgtgccctagtttgcttttccaaccaagactcttctggattaacttttttatgcgtttgttgatctgtttgctggatatttcttgcttcctttcttctttccttttctgaaactctcaaattgttacaaaccagaattaatagagagttagaacaatatatttcgtattcaa

SEQ ID NO: 28 - DNA, 14925 bp, Artificial Sequence
  Synthetic Construct
ggtaccttcctaggatactagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttgcctcagcaagctatcaaagtcggaacgctcaacgattgcccctcacgaggggaccagttgacaatccagccaatttcgataattccttaattaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaactagtgatctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgttgtcaatcaattggcaagtcataaaatgcattaaaaaatattttcatactcaactacaaatccatgagtataactataattataaagcaatgattagaatctgacaaggattctggaaaattacataaaggaaagttcataaatgtctaaaacacaagaggacatacttgtattcagtaacatttgcagcttttctaggtctgaaaatatatttgttgcctagtgaataagcataatggtacaactacaagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcaaactcagtaggattctggtgtgtgcgcaatgaaactgatgcattgaacttgacgaacgttgtcgaaaccgatgatacgaacgaaagctcttcacttgtcgtcgtcatccttatagtctttgtcatcgtcatccttatagtcggagccagaacccaaggagactttacgcttctttttaggatcctctacctttcgctttttcttcggtccggatccggacgtttgggacggctcttgagcgggagtctgatcctcacgttcagccgggggtgcctttgacttgctagctttcttcgtctttctcgcgggggctgtcccttgtgcatctctaggctcctctctcttcggcattgttttgccggtgagcgccacttgagtcaagttatgtgtcgctacgtccagatctgcgttgcatgtcttaccgcaactgagacactgaaaggcttctccatccctgttccccacttcacagtgcccgcatttcggacaggtgattgatgttctctctggacgcacctcgaacacgtagaaggatcggtgggtccttaagtcagaaaacgccttatgtagcccttggataaaccatcggttttccttctttgccgtgaagaagttatcccagcccgggagtcgtttgccagacccgtggaaaaatcggacatttagatcttctataacggggatcacaatttggcattgggttcgccttctagttttttctatcacgtaattaatggacctcctacacaattcttctttacgtcgtgacaactttagatattctggagaggtacgcttcaaagcccaaagagcctcatttttcaacttttgtgcttctactgagagtcgtggtttgtacgctcgcgcccatgtcctgtcttttcgcatcacttccaccggtgctttcttcttggctcctttctttggggccggggtaaagaatacttggtcacgggatacgctgttggaaaggagcgcctcgcttatgaacgtggtattgctactcattttatcccacggtagtcgctttgggtccagaccgaagtccgcgcacaactgtgtacgcgcggtctctttggagaccccgtccagagctctgacctctgcttgttgtgcctctgggagtgcctcaacggatctggccctgagttcctcctcgttacggtcccatgcaattcggtacgcagaaatatctttcaaaaggtcatcaggcaatgtaaaacgatcaagcggttcgcactggagggccccgttctcctgtgtgacacgactgatacccgcgctaattgggtttgtttgtcccagatctatagcaactaaggccacagtttgtgtcgctgtcagtttatctaaggttgcttttgtctgttttcccttgagagtccattttctagcataagttccgcaagcgtccaaggtataggtgaaggtcacgatgtttcgtctgacatcgatgactggatcaccggtaaataaatctagaagtgcatttaaacttatgtccttgggagcaatagtcctccacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacctggcgtttcttagtaatcctcttacgtctatgactacccaatcggtacctatccttacagttacgagaagtgcatcttgagccttctccttttctgacctccagtacctacgcatacgtttattcttcttcgggctaagtcctggaaccgtcactgcttttcctgtcttgggactaattgccgtaccagcttccctctgccactcgggtatatatcccgggcaacccttctgtatgtcacaacggttcctgaccacgcccagtggaatcggagcgttcggatccctgacgtaaccggcgtactcgggtggtaacactatttcgtcccttggtcggtaagcctgcggagatatggtctggtagacgtagaatgatgggtttatgcctggcggctgtagtagatgacctgtttcatttgtagctacctcctcctcctcggccttgatttccggcaatccttcatcagctcgtgaggcgtttatgctttccaatctagctcgggccttctcatttcgaagctgtactttctttaaaacgccatcgtatcgtcctagcgtgtggtcaaagatcaggttaagaccttgtacatgagaatagccgtgattgctcacccctgtagccgcgaaccaggccgcgtgactttctgagctcttacccggtttgcaggcagctcgctctgtagtgctgagcgcatagatgtacctctgtatggcttcagaggctttcattatgggccattctgcgaaatccctgcttttggtgaccacgtggcacttcgccggcggctgaaaattcggcggttcctcctcactctttccctgcaagtacgccactacagcttcctcgccttgtgctgccagaattttaccccccctcttcatatagctagacctaaaacgctccccaggaaagtgtttcttgaggactttggaaaactctgattcgacggcgggcttcggcatctgttaatcagaaaaactcagattaatcgacaaattcgatcgcacaaactagaaactaacaccagatctagatagaaatcacaaatcgaagagtaattattcgacaaaactcaaattatttgaacaaatcggatgatatctatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttaccttgatcacggtagagagaattgagagaaagtttttaagattttgagaaattgaaatctgaattgtgaagaagaagagctctttgggtattgttttatagaagaagaagaagaaaagacgaggacgactaggtcacgagaaagctaaggcggtgaagcaatagctaataataaaatgacacgtgtattgagcgttgtttacacgcaaagttgtttttggctaattgccttatttttaggttgaggaaaagtatttgtgctttgagttgataaacacgactcgtgtgtgccggctgcaaccactttgacgccgtttattactgactcgtcgacaaccacaatttctaacggtcgtcataagatccagccgttgagatttaacgatcgttacgatttatatttttttagcattatcgttttattttttaaatatacggtggagctgaaaattggcaataattgaaccgtgggtcccactgcattgaagcgtatttcgtattttctagaattcttcgtgctttatttcttttcctttttgtttttttttgccatttatctaatgcaagtgggcttataaaatcagtgaatttcttggaaaagtaacttctttatcgtataacatattgtgaaattatccatttcttttaattttttagtgttattggatatttttgtatgattattgatttgcataggataatgacttttgtatcaagttggtgaacaagtctcgttaaaaaaggcaagtggtttggtgactcgatttattcttgttatttaattcatatatcaatggatcttatttggggcctggtccatatttaacactcgtgttcagtccaatgaccaataatattttttcattaataacaatgtaacaagaatgatacacaaaacattctttgaataagttcgctatgaagaagggaacttatccggtcctagatcatcagttcatacaaacctccatagagttcaacatcttaaacaagaatatcctgatctgaagaatgtggaggctttagtcccttggatacttgggaggctgtggaagaacagaaacgagctggtgcttaaagggagggaatttggaaccaatgaggtattagtaaggacacaagaagatgcagatgagtggattagaaggaaagaggctcagaatgtaaggaaagcaccaaccacaacaccgtcgggacagacgagaaacatgggaagctccaccacaatcttgggttaagtgcaactttgatggggcatggccaacagaaggattaaaatgtggcttagggtgggtgcttcgcgatcatacagggaaggtgttatggttaggtgcaagagctgtggtaaaagtaagaagcgtgctggaagtagaagtggaggctcttagatgggctgtgctgtcattatcccgattcaattataggaagatcatttttgaggtggattctcagcaacttgtgtctttggttacatgaaagttatgcttgtcaagtctcaatccaattatccaagacataaagtatctacttagcaagtttgaggattttatgcttgtgcatacaagccgagaaggaaatggagtggcagatagaatagctaaggaatctctttcttttgagaattatgatctaaagttgtattctattgtaccaatttgggttaaaagctctgttgagctagact

SEQ ID NO: 29 - DNA, 465 bp, Artificial Sequence
  Synthetic Construct
tggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagctt

SEQ ID NO: 30 - DNA, 191 bp, Artificial Sequence
  Synthetic Construct
ctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 31 - DNA, 20 bp, Artificial Sequence
  Synthetic Construct
cagttgacaatccagccaat

SEQ ID NO: 32 - DNA, 36 bp, Artificial Sequence
  Synthetic Construct
gtcggaacgctcaacgattgcccctcacgaggggac

SEQ ID NO: 33 - DNA, 15314 bp, Artificial Sequence
  Synthetic Construct
ggtaccttcctaggatactaggccagtgccaagcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatcccgcctcagcaagctatcaaagtcggaacgctcaacgattgcccctcacgaggggaccagttgacaatccagccaatttcgataattccttaattaaatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagactagtgatctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgttgtcaatcaattggcaagtcataaaatgcattaaaaaatattttcatactcaactacaaatccatgagtataactataattataaagcaatgattagaatctgacaaggattctggaaaattacataaaggaaagttcataaatgtctaaaacacaagaggacatacttgtattcagtaacatttgcagcttttctaggtctgaaaatatatttgttgcctagtgaataagcataatggtacaactacaagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcaaactcagtaggattctggtgtgtgcgcaatgaaactgatgcattgaacttgacgaacgttgtcgaaaccgatgatacgaacgaaagctcttcacttgtcgtcgtcatccttatagtctttgtcatcgtcatccttatagtcggagccagaacccaaggagactttacgcttctttttaggatcctctacctttcgctttttcttcggtccggatccggacgtttgggacggctcttgagcgggagtctgatcctcacgttcagccgggggtgcctttgacttgctagctttcttcgtctttctcgcgggggctgtcccttgtgcatctctaggctcctctctcttcggcattgttttgccggtgagcgccacttgagtcaagttatgtgtcgctacgtccagatctgcgttgcatgtcttaccgcaactgagacactgaaaggcttctccatccctgttccccacttcacagtgcccgcatttcggacaggtgattgatgttctctctggacgcacctcgaacacgtagaaggatcggtgggtccttaagtcagaaaacgccttatgtagcccttggataaaccatcggttttccttctttgccgtgaagaagttatcccagcccgggagtcgtttgccagacccgtggaaaaatcggacatttagatcttctataacggggatcacaatttggcattgggttcgccttctagttttttctatcacgtaattaatggacctcctacacaattcttctttacgtcgtgacaactttagatattctggagaggtacgcttcaaagcccaaagagcctcatttttcaacttttgtgcttctactgagagtcgtggtttgtacgctcgcgcccatgtcctgtcttttcgcatcacttccaccggtgctttcttcttggctcctttctttggggccggggtaaagaatacttggtcacgggatacgctgttggaaaggagcgcctcgcttatgaacgtggtattgctactcattttatcccacggtagtcgctttgggtccagaccgaagtccgcgcacaactgtgtacgcgcggtctctttggagaccccgtccagagctctgacctctgcttgttgtgcctctgggagtgcctcaacggatctggccctgagttcctcctcgttacggtcccatgcaattcggtacgcagaaatatctttcaaaaggtcatcaggcaatgtaaaacgatcaagcggttcgcactggagggccccgttctcctgtgtgacacgactgatacccgcgctaattgggtttgtttgtcccagatctatagcaactaaggccacagtttgtgtcgctgtcagtttatctaaggttgcttttgtctgttttcccttgagagtccattttctagcataagttccgcaagcgtccaaggtataggtgaaggtcacgatgtttcgtctgacatcgatgactggatcaccggtaaataaatctagaagtgcatttaaacttatgtccttgggagcaatagtcctccacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacctggcgtttcttagtaatcctcttacgtctatgactacccaatcggtacctatccttacagttacgagaagtgcatcttgagccttctccttttctgacctccagtacctacgcatacgtttattcttcttcgggctaagtcctggaaccgtcactgcttttcctgtcttgggactaattgccgtaccagcttccctctgccactcgggtatatatcccgggcaacccttctgtatgtcacaacggttcctgaccacgcccagtggaatcggagcgttcggatccctgacgtaaccggcgtactcgggtggtaacactatttcgtcccttggtcggtaagcctgcggagatatggtctggtagacgtagaatgatgggtttatgcctggcggctgtagtagatgacctgtttcatttgtagctacctcctcctcctcggccttgatttccggcaatccttcatcagctcgtgaggcgtttatgctttccaatctagctcgggccttctcatttcgaagctgtactttctttaaaacgccatcgtatcgtcctagcgtgtggtcaaagatcaggttaagaccttgtacatgagaatagccgtgattgctcacccctgtagccgcgaaccaggccgcgtgactttctgagctcttacccggtttgcaggcagctcgctctgtagtgctgagcgcatagatgtacctctgtatggcttcagaggctttcattatgggccattctgcgaaatccctgcttttggtgaccacgtggcacttcgccggcggctgaaaattcggcggttcctcctcactctttccctgcaagtacgccactacagcttcctcgccttgtgctgccagaattttaccccccctcttcatatagctagacctaaaacgctccccaggaaagtgtttcttgaggactttggaaaactctgattcgacggcgggcttcggcatctgttaatcagaaaaactcagattaatcgacaaattcgatcgcacaaactagaaactaacaccagatctagatagaaatcacaaatcgaagagtaattattcgacaaaactcaaattatttgaacaaatcggatgatatctatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttaccttgatcacggtagagagaattgagagaaagtttttaagattttgagaaattgaaatctgaattgtgaagaagaagagctctttgggtattgttttatagaagaagaagaagaaaagacgaggacgactaggtcacgagaaagctaaggcggtgaagcaatagctaataataaaatgacacgtgtattgagcgttgtttacacgcaaagttgtttttggctaattgccttatttttaggttgaggaaaagtatttgtgctttgagttgataaacacgactcgtgtgtgccggctgcaaccactttgacgccgtttattactgactcgtcgacaaccacaatttctaacggtcgtcataagatccagccgttgagatttaacgatcgttacgatttatatttttttagcattatcgttttattttttaaatatacggtggagctgaaaattggcaataattgaaccgtgggtcccactgcattgaagcgtatttcgtattttctagaattcttcgtgctttatttcttttcctttttgtttttttttgccatttatctaatgcaagtgggcttataaaatcagtgaatttcttggaaaagtaacttctttatcgtataacatattgtgaaattatccatttcttttaattttttagtgttattggatatttttgtatgattattgatttgcataggataatgacttttgtatcaagttggtgaacaagtctcgttaaaaaaggcaagtggtttggtgactcgatttattcttgttatttaattcatatatcaatggatcttatttggggcctggtccatatttaacactcgtgttcagtccaatgaccaataatattttttcattaataacaatgtaacaagaatgatacacaaaacattctttgaataagttcgctatgaagaagggaacttatccggtcctagatcatcagttcatacaaacctccatagagttcaacatcttaaacaagaatatcctgatctgaagaatgtggaggctttagtcccttggatacttgggaggctgtggaagaacagaaacgagctggtgcttaaagggagggaatttggaaccaatgaggtattagtaaggacacaagaagatgcagatgagtggattagaaggaaagaggctcagaatgtaaggaaagcaccaaccacaacaccgtcgggacagacgagaaacatgggaagctccaccacaatcttgggttaagtgcaactttgatggggcatggccaacagaaggattaaaatgtggcttagggtgggtgcttcgcgatcatacagggaaggtgttatggttaggtgcaagagctgtggtaaaagtaagaagcgtgctggaagtagaagtggaggctcttagatgggctgtgctgtcattatcccgattcaattataggaagatcatttttgaggtggattctcagcaacttgtgtctttggttacatgaaagttatgcttgtcaagtctcaatccaattatccaagacataaagtatctacttagcaagtttgaggattttatgcttgtgcatacaagccgagaaggaaatggagtggcagatagaatagctaaggaatctctttcttttgagaattatgatctaaagttgtattctattgtaccaatttgggttaaaagctctgttgagctagact

SEQ ID NO: 34 - DNA, 769 bp, Artificial Sequence
  Synthetic Construct
ggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccc

SEQ ID NO: 35 - DNA, 250 bp, Artificial Sequence
  Synthetic Construct
atatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 36 - DNA, 20 bp, Artificial Sequence
  Synthetic Construct
cagttgacaatccagccaat

SEQ ID NO: 37 - DNA, 36 bp, Artificial Sequence; identical to SEQ ID NO: 32
  Synthetic Construct

SEQ ID NO: 38 - DNA, 18926 bp, Artificial Sequence
  Synthetic Construct
ggtaccttcctaggatactagaagttgtaatgagttgctggcctctcttgggattgttcatcaaagtagctgtccctacacaccacaacagaatggtgttgtggagagaaagcacagacacatccttgagatggcaagggcacttaagtttcagagtggtgtacctaccaggttttggggagactgtgtcaagactgttgtatacctgatcaatagactccctactccaatcttacaaggcaaatgtccatatgaactactctatcagaaacatgccaagcttgatcacttgagagtgtttgggtgtcttgcttttgcagcaacactgcccaaaggtgacaaacttgcacctagagccaagcgaaccatcttcattggttattctgagactcaaaagggttacaggttgtatgacttggataataaggtgatcatagtgagcagggatgtagttttcagagagtttcagttccctttcaaagagtccatctcccatgaacctgatatgttcactcgagcaggttcacttagctctgaagatacagctttgcagctagtgtataatgatatttttccttccagctttcatactgatgatgatgatactccagttcttattcctgacattgtagaggccaatgaggatatcactttggggactcattctacccaaaatcatatcacttcagctgatgctgcaccttcaggccacctgcatgctcctgcagagtcagttgagtcagctgagcctgacctagcaaatgctgaatttgagcaccatactgctgtagctgatccctctctagttccagcaaacctgcacactagacctaaacgcaatgcaggtcctcccatctggctcaaggactttgtgacactgaacaaaggctctagggatgttccataccctatagccaactatgtttcctatgatcacttgcctgttcactatcaagcttatttgagtgctttctccacttatactgaacctaaatccttcaaagaagcagctcaagatgagaaatggatggaggccatgtcccttgagatacaggctcttgaggataataacacctgggagattgtccctttaccccctggtaaacagcctatagggtccaaatgggtgtacaaaattaaatacaaagctaatggtgaagttgacaggtttaaggcaaggctagtggccaagggatacactcagcaagaaggccttgactaccatgaaactttttctccagtggccaaaatggtcactgtaaaagctgtcatatctgttgctgcttccaagggctggttccttttccagatggatgtcaacaatgcttttctacaaggtgacctcatggaagaagtttatatgtctctgcctcagggttttcacagccaaggggagaccaaagtgtgcaggctgttgaatccctctatggtttaaagcaagcatcaaggcagtggaacatcaagcttaccactgtccttatgcaggctggttttatgcaaagtgcttatgatcactccttgtttaccaaaagagaggggactgaccttgtcataatcctgatttatgtggatgatctgttaataacaggcagcagcaatgttttgatttcagaagcaaaggcaaccctgcatcagcatttcaagatgaaagatttgggagaactaaaatacttcttaggcattgaggtgctgagatcagaaaagggaatcctactgaaccagaggaagtatgcactggaactgatatcaggtgttggtctgggaggctgcagaccagtgtcaaccccaatggaacagaaccaaaggctgactactgtagaatatgacaagcacttaggaaagactgatgatgcagagttagaggatgtgggttcctatcagagactggtgggaaagcttctctacttgacaatcacaaggccagatatcagctttgcagtgcaggtgctgagtcaattcatgcagcaacccaaacagtcacacctggaagcagcattgagggttgtgaagtacataaaaggttctccaggtgttaggaacgcggccgcatgcatatgagtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggcctcagcaagctatcaaagtcggaacgctcaacgattgcccctcacgaggggaccagttgacaatccagccaatttcgataattccttaattaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacactagtgatctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgttgtcaatcaattggcaagtcataaaatgcattaaaaaatattttcatactcaactacaaatccatgagtataactataattataaagcaatgattagaatctgacaaggattctggaaaattacataaaggaaagttcataaatgtctaaaacacaagaggacatacttgtattcagtaacatttgcagcttttctaggtctgaaaatatatttgttgcctagtgaataagcataatggtacaactacaagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcaaactcagtaggattctggtgtgtgcgcaatgaaactgatgcattgaacttgacgaacgttgtcgaaaccgatgatacgaacgaaagctcttcacttgtcgtcgtcatccttatagtctttgtcatcgtcatccttatagtcggagccagaacccaaggagactttacgcttctttttaggatcctctacctttcgctttttcttcggtccggatccggacgtttgggacggctcttgagcgggagtctgatcctcacgttcagccgggggtgcctttgacttgctagctttcttcgtctttctcgcgggggctgtcccttgtgcatctctaggctcctctctcttcggcattgttttgccggtgagcgccacttgagtcaagttatgtgtcgctacgtccagatctgcgttgcatgtcttaccgcaactgagacactgaaaggcttctccatccctgttccccacttcacagtgcccgcatttcggacaggtgattgatgttctctctggacgcacctcgaacacgtagaaggatcggtgggtccttaagtcagaaaacgccttatgtagcccttggataaaccatcggttttccttctttgccgtgaagaagttatcccagcccgggagtcgtttgccagacccgtggaaaaatcggacatttagatcttctataacggggatcacaatttggcattgggttcgccttctagttttttctatcacgtaattaatggacctcctacacaattcttctttacgtcgtgacaactttagatattctggagaggtacgcttcaaagcccaaagagcctcatttttcaacttttgtgcttctactgagagtcgtggtttgtacgctcgcgcccatgtcctgtcttttcgcatcacttccaccggtgctttcttcttggctcctttctttggggccggggtaaagaatacttggtcacgggatacgctgttggaaaggagcgcctcgcttatgaacgtggtattgctactcattttatcccacggtagtcgctttgggtccagaccgaagtccgcgcacaactgtgtacgcgcggtctctttggagaccccgtccagagctctgacctctgcttgttgtgcctctgggagtgcctcaacggatctggccctgagttcctcctcgttacggtcccatgcaattcggtacgcagaaatatctttcaaaaggtcatcaggcaatgtaaaacgatcaagcggttcgcactggagggccccgttctcctgtgtgacacgactgatacccgcgctaattgggtttgtttgtcccagatctatagcaactaaggccacagtttgtgtcgctgtcagtttatctaaggttgcttttgtctgttttcccttgagagtccattttctagcataagttccgcaagcgtccaaggtataggtgaaggtcacgatgtttcgtctgacatcgatgactggatcaccggtaaataaatctagaagtgcatttaaacttatgtccttgggagcaatagtcctccacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacctggcgtttcttagtaatcctcttacgtctatgactacccaatcggtacctatccttacagttacgagaagtgcatcttgagccttctccttttctgacctccagtacctacgcatacgtttattcttcttcgggctaagtcctggaaccgtcactgcttttcctgtcttgggactaattgccgtaccagcttccctctgccactcgggtatatatcccgggcaacccttctgtatgtcacaacggttcctgaccacgcccagtggaatcggagcgttcggatccctgacgtaaccggcgtactcgggtggtaacactatttcgtcccttggtcggtaagcctgcggagatatggtctggtagacgtagaatgatgggtttatgcctggcggctgtagtagatgacctgtttcatttgtagctacctcctcctcctcggccttgatttccggcaatccttcatcagctcgtgaggcgtttatgctttccaatctagctcgggccttctcatttcgaagctgtactttctttaaaacgccatcgtatcgtcctagcgtgtggtcaaagatcaggttaagaccttgtacatgagaatagccgtgattgctcacccctgtagccgcgaaccaggccgcgtgactttctgagctcttacccggtttgcaggcagctcgctctgtagtgctgagcgcatagatgtacctctgtatggcttcagaggctttcattatgggccattctgcgaaatccctgcttttggtgaccacgtggcacttcgccggcggctgaaaattcggcggttcctcctcactctttccctgcaagtacgccactacagcttcctcgccttgtgctgccagaattttaccccccctcttcatatagctagacctaaaacgctccccaggaaagtgtttcttgaggactttggaaaactctgattcgacggcgggcttcggcatctgttaatcagaaaaactcagattaatcgacaaattcgatcgcacaaactagaaactaacaccagatctagatagaaatcacaaatcgaagagtaattattcgacaaaactcaaattatttgaacaaatcggatgatatctatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttaccttgatcacggtagagagaattgagagaaagtttttaagattttgagaaattgaaatctgaattgtgaagaagaagagctctttgggtattgttttatagaagaagaagaagaaaagacgaggacgactaggtcacgagaaagctaaggcggtgaagcaatagctaataataaaatgacacgtgtattgagcgttgtttacacgcaaagttgtttttggctaattgccttatttttaggttgaggaaaagtatttgtgctttgagttgataaacacgactcgtgtgtgccggctgcaaccactttgacgccgtttattactgactcgtcgacaaccacaatttctaacggtcgtcataagatccagccgttgagatttaacgatcgttacgatttatatttttttagcattatcgttttattttttaaatatacggtggagctgaaaattggcaataattgaaccgtgggtcccactgcattgaagcgtatttcgtattttctagaattcttcgtgctttatttcttttcctttttgtttttttttgccatttatctaatgcaagtgggcttataaaatcagtgaatttcttggaaaagtaacttctttatcgtataacatattgtgaaattatccatttcttttaattttttagtgttattggatatttttgtatgattattgatttgcataggataatgacttttgtatcaagttggtgaacaagtctcgttaaaaaaggcaagtggtttggtgactcgatttattcttgttatttaattcatatatcaatggatcttatttggggcctggtccatatttaacactcgtgttcagtccaatgaccaataatattttttcattaataacaatgtaacaagaatgatacacaaaacattctttgaataagttcgctatgaagaagggaacttatccggtcctagatcatcagttcatacaaacctccatagagttcaacatcttaaacaagaatatcctgatctgaagaatgtggaggctttagtcccttggatacttgggaggctgtggaagaacagaaacgagctggtgcttaaagggagggaatttggaaccaatgaggtattagtaaggacacaagaagatgcagatgagtggattagaaggaaagaggctcagaatgtaaggaaagcaccaaccacaacaccgtcgggacagacgagaaacatgggaagctccaccacaatcttgggttaagtgcaactttgatggggcatggccaacagaaggattaaaatgtggcttagggtgggtgcttcgcgatcatacagggaaggtgttatggttaggtgcaagagctgtggtaaaagtaagaagcgtgctggaagtagaagtggaggctcttagatgggctgtgctgtcattatcccgattcaattataggaagatcatttttgaggtggattctcagcaacttgtgtctttggttacatgaaagttatgcttgtcaagtctcaatccaattatccaagacataaagtatctacttagcaagtttgaggattttatgcttgtgcatacaagccgagaaggaaatggagtggcagatagaatagctaaggaatctctttcttttgagaattatgatctaaagttgtattctattgtaccaatttgggttaaaagctctgttgagctagact

SEQ ID NO: 39 - DNA, 1986 bp, Artificial Sequence
  Synthetic Construct
agtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacag

SEQ ID NO: 40 - DNA, 635 bp, Artificial Sequence
  Synthetic Construct
agagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaac

SEQ ID NO: 41 - DNA, 20 bp, Artificial Sequence
  Synthetic Construct
cagttgacaatccagccaat

SEQ ID NO: 42 - DNA, 36 bp, Artificial Sequence; identical to SEQ ID NO: 32
  Synthetic Construct

SEQ ID NO: 43 - DNA, 2018 bp, Artificial Sequence
  Synthetic Construct
aagttgtaatgagttgctggcctctcttgggattgttcatcaaagtagctgtccctacacaccacaacagaatggtgttgtggagagaaagcacagacacatccttgagatggcaagggcacttaagtttcagagtggtgtacctaccaggttttggggagactgtgtcaagactgttgtatacctgatcaatagactccctactccaatcttacaaggcaaatgtccatatgaactactctatcagaaacatgccaagcttgatcacttgagagtgtttgggtgtcttgcttttgcagcaacactgcccaaaggtgacaaacttgcacctagagccaagcgaaccatcttcattggttattctgagactcaaaagggttacaggttgtatgacttggataataaggtgatcatagtgagcagggatgtagttttcagagagtttcagttccctttcaaagagtccatctcccatgaacctgatatgttcactcgagcaggttcacttagctctgaagatacagctttgcagctagtgtataatgatatttttccttccagctttcatactgatgatgatgatactccagttcttattcctgacattgtagaggccaatgaggatatcactttggggactcattctacccaaaatcatatcacttcagctgatgctgcaccttcaggccacctgcatgctcctgcagagtcagttgagtcagctgagcctgacctagcaaatgctgaatttgagcaccatactgctgtagctgatccctctctagttccagcaaacctgcacactagacctaaacgcaatgcaggtcctcccatctggctcaaggactttgtgacactgaacaaaggctctagggatgttccataccctatagccaactatgtttcctatgatcacttgcctgttcactatcaagcttatttgagtgctttctccacttatactgaacctaaatccttcaaagaagcagctcaagatgagaaatggatggaggccatgtcccttgagatacaggctcttgaggataataacacctgggagattgtccctttaccccctggtaaacagcctatagggtccaaatgggtgtacaaaattaaatacaaagctaatggtgaagttgacaggtttaaggcaaggctagtggccaagggatacactcagcaagaaggccttgactaccatgaaactttttctccagtggccaaaatggtcactgtaaaagctgtcatatctgttgctgcttccaagggctggttccttttccagatggatgtcaacaatgcttttctacaaggtgacctcatggaagaagtttatatgtctctgcctcagggttttcacagccaaggggagaccaaagtgtgcaggctgttgaatccctctatggtttaaagcaagcatcaaggcagtggaacatcaagcttaccactgtccttatgcaggctggttttatgcaaagtgcttatgatcactccttgtttaccaaaagagaggggactgaccttgtcataatcctgatttatgtggatgatctgttaataacaggcagcagcaatgttttgatttcagaagcaaaggcaaccctgcatcagcatttcaagatgaaagatttgggagaactaaaatacttcttaggcattgaggtgctgagatcagaaaagggaatcctactgaaccagaggaagtatgcactggaactgatatcaggtgttggtctgggaggctgcagaccagtgtcaaccccaatggaacagaaccaaaggctgactactgtagaatatgacaagcacttaggaaagactgatgatgcagagttagaggatgtgggttcctatcagagactggtgggaaagcttctctacttgacaatcacaaggccagatatcagctttgcagtgcaggtgctgagtcaattcatgcagcaacccaaacagtcacacctggaagcagcattgagggttgtgaagtacataaaaggttctccaggtgttaggaa

SEQ ID NO: 44 - DNA, 5861 bp, Artificial Sequence
  Synthetic Construct
agtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatggatcattatcttgatattagacttagacctgatccagaatttccaccagctcaacttatgtctgttctttttggaaaacttcatcaagctcttgttgctcaaggaggagatagaattggagtttcttttcctgatcttgatgaatcaagatcaagacttggagaaagacttagaattcatgcttctgctgatgatcttagagctttgcttgctagaccttggcttgaaggacttagagatcatcttcaatttggagaaccagctgttgttccacatccaactccttatagacaagtttcaagagttcaagctaaatctaatccagaaagacttagaagaagacttatgagaagacatgatctttctgaagaagaagctagaaaaagaattcctgatactgttgctagagctttggatttgccttttgttacacttagatcacaatctactggacaacattttagactttttattagacatggaccacttcaagttactgctgaagaaggaggatttacttgttatggactttctaagggaggttttgttccttggttttatccctatgacgtgcccgattatgccggatctggagctactaatttttctcttcttaagcaagctggagatgttgaagaaaatcctggacccatgccgaagcccgccgtcgaatcagagttttccaaagtcctcaagaaacactttcctggggagcgttttaggtctagctatatgaagagggggggtaaaattctggcagcacaaggcgaggaagctgtagtggcgtacttgcagggaaagagtgaggaggaaccgccgaattttcagccgccggcgaagtgccacgtggtcaccaaaagcagggatttcgcagaatggcccataatgaaagcctctgaagccatacagaggtacatctatgcgctcagcactacagagcgagctgcctgcaaaccgggtaagagctcagaaagtcacgcggcctggttcgcggctacaggggtgagcaatcacggctattctcatgtacaaggtcttaacctgatctttgaccacacgctaggacgatacgatggcgttttaaagaaagtacagcttcgaaatgagaaggcccgagctagattggaaagcataaacgcctcacgagctgatgaaggattgccggaaatcaaggccgaggaggaggaggtagctacaaatgaaacaggtcatctactacagccgccaggcataaacccatcattctacgtctaccagaccatatctccgcaggcttaccgaccaagggacgaaatagtgttaccacccgagtacgccggttacgtcagggatccgaacgctccgattccactgggcgtggtcaggaaccgttgtgacatacagaagggttgcccgggatatatacccgagtggcagagggaagctggtacggcaattagtcccaagacaggaaaagcagtgacggttccaggacttagcccgaagaagaataaacgtatgcgtaggtactggaggtcagaaaaggagaaggctcaagatgcacttctcgtaactgtaaggataggtaccgattgggtagtcatagacgtaagaggattactaagaaacgccaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtggaggactattgctcccaaggacataagtttaaatgcacttctagatttatttaccggtgatccagtcatcgatgtcagacgaaacatcgtgaccttcacctataccttggacgcttgcggaacttatgctagaaaatggactctcaagggaaaacagacaaaagcaaccttagataaactgacagcgacacaaactgtggccttagttgctatagatctgggacaaacaaacccaattagcgcgggtatcagtcgtgtcacacaggagaacggggccctccagtgcgaaccgcttgatcgttttacattgcctgatgaccttttgaaagatatttctgcgtaccgaattgcatgggaccgtaacgaggaggaactcagggccagatccgttgaggcactcccagaggcacaacaagcagaggtcagagctctggacggggtctccaaagagaccgcgcgtacacagttgtgcgcggacttcggtctggacccaaagcgactaccgtgggataaaatgagtagcaataccacgttcataagcgaggcgctcctttccaacagcgtatcccgtgaccaagtattctttaccccggccccaaagaaaggagccaagaagaaagcaccggtggaagtgatgcgaaaagacaggacatgggcgcgagcgtacaaaccacgactctcagtagaagcacaaaagttgaaaaatgaggctctttgggctttgaagcgtacctctccagaatatctaaagttgtcacgacgtaaagaagaattgtgtaggaggtccattaattacgtgatagaaaaaactagaaggcgaacccaatgccaaattgtgatccccgttatagaagatctaaatgtccgatttttccacgggtctggcaaacgactcccgggctgggataacttcttcacggcaaagaaggaaaaccgatggtttatccaagggctacataaggcgttttctgacttaaggacccaccgatccttctacgtgttcgaggtgcgtccagagagaacatcaatcacctgtccgaaatgcgggcactgtgaagtggggaacagggatggagaagcctttcagtgtctcagttgcggtaagacatgcaacgcagatctggacgtagcgacacataacttgactcaagtggcgctcaccggcaaaacaatgccgaagagagaggagcctagagatgcacaagggacagcccccgcgagaaagacgaagaaagctagcaagtcaaaggcacccccggctgaacgtgaggatcagactcccgctcaagagccgtcccaaacgtccggatccggaccgaagaaaaagcgaaaggtagaggatcctaaaaagaagcgtaaagtctccttgggttctggctccgactataaggatgacgatgacaaagactataaggatgacgacgacaagtgaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaac

SEQ ID NO: 45 - DNA, 21 bp, Artificial Sequence
  Synthetic Construct
ggctctcaaataattgcagga

SEQ ID NO: 46 - DNA, 52 bp, Arabidopsis thaliana
ggctctcaaataattgcaggattggctggattgtcaactgcaaagtacctgg

SEQ ID NO: 47 - protein, 11 aa, Arabidopsis thaliana
GLAGLSTAKYL

SEQ ID NO: 48 - DNA, 21 bp, Artificial Sequence
  Synthetic Construct
ggctctcaaataattgcagga

SEQ ID NO: 49 - DNA, 33 bp, Arabidopsis thaliana
attgcaggattggctggattgtcaactgcaaag

SEQ ID NO: 50 - protein, 10 aa, Arabidopsis thaliana
GLAGLSTAKY

SEQ ID NO: 51 - DNA, 27 bp, Artificial Sequence
  Synthetic Construct
attgcaggaggattgtcaactgcaaag

SEQ ID NO: 52 - DNA, 27 bp, Artificial Sequence
  Synthetic Construct
attgcaggaggattgtcaactgcaaag

SEQ ID NO: 53 - DNA, 27 bp, Artificial Sequence
  Synthetic Construct
attgcaggaggattgtcaactgcaaag

SEQ ID NO: 54 - DNA, 27 bp, Artificial Sequence
  Synthetic Construct
attgcaggaggattgtcaactgcaaag

SEQ ID NO: 55 - DNA, 27 bp, Artificial Sequence
  Synthetic Construct
attgcaggaggattgtcaactgcaaag

SEQ ID NO: 56 - DNA, 27 bp, Artificial Sequence
  Synthetic Construct
attgcaggaggattgtcaactgcaaag

SEQ ID NO: 57 - protein, 28 aa, Artificial Sequence
  Synthetic Construct
FRASPRPLKPLNVVIAGAGLAGLSTAKY

SEQ ID NO: 58 - protein, 28 aa, Arabidopsis thaliana
FRSAPRPAKPLKVVIAGAGLAGLSTAKY

SEQ ID NO: 59 - protein, 28 aa, Chlamydomonoas reinhardtii
LKDAPRPAKPLKVVIAGAGLAGLSAAKY

SEQ ID NO: 60 - protein, 28 aa, Physcomitrella patens
MRDSPRPEKPLRVVIAGAGLAGLSTAKY

SEQ ID NO: 61 - protein, 28 aa, Brachypodium distachyon
FRSSERPSKPLQVVIAGAGLAGLSTAKY

SEQ ID NO: 62 - protein, 28 aa, Oryza sativa
FRNSEQPTKPLQVVIAGAGLAGLSTAKY

SEQ ID NO: 63 - protein, 28 aa, Vitis vinifera
FHTSPRPSKPLEVVIAGAGLAGLSTAKY

SEQ ID NO: 64 - protein, 28 aa, Glycine max
FRASPRPLKPLNIVIAGAGLAGLSTAKY

SEQ ID NO: 65 - protein, 28 aa, Glycine max
FRASPRPLKPLNIVIAGAGLAGLSTAKY

SEQ ID NO: 66 - protein, 28 aa, Glycine max
FRASPRPLKPLNIVIAGAGLAGLSTAKY

SEQ ID NO: 67 - protein, 28 aa, Solanum lycopersicum
FRASPRPTKPLEIVIAGAGLGGLSTAKY

SEQ ID NO: 68 - DNA, 114 bp, Arabidopsis thaliana
tgctttgtttgggatctgatggaattaatgttcatgtgatgaagttcttttggctctcaaataattgcaggattggctggattgtcaactgcaaagtacctggctgatgcaggc

SEQ ID NO: 69 - protein, 15 aa, Arabidopsis thaliana
GLAGLSTAKYLADAG

SEQ ID NO: 70 - DNA, 91 bp, Artificial Sequence
  Synthetic Construct
tgctttgtttgggatctgatggaattaatgttcatgtgatgaagttcttttggctctcaaataattgcaggattgggagtgttgtctgctg

SEQ ID NO: 71 - DNA, 114 bp, Artificial Sequence
  Synthetic Construct
tgctttgtttgggatctgatggaattaatgttcatgtgatgaagttcttttggctctcaaataattgcaggattgtcaactgcaaagtacctggctgatgcgggccacaaacct

SEQ ID NO: 72 - DNA, 114 bp, Artificial Sequence
  Synthetic Construct
  misc_feature 81, 82: n = A,T,C or G
tgctttgtttgggatctgatggaattaatgttcatgtgatgaagttcttttggctctcaaataattgcaggattggctggnntgtcaaacctggatgatgcgggtgatgcacct

SEQ ID NO: 73 - DNA, 115 bp, Artificial Sequence
  Synthetic Construct
tgctttgtttggggatctgatggaattaatgttcatgtgatgaagttcttttggctctcaaataattgcaggatggattggattgtgcactgcacctgactgaggtgaggcacaa

SEQ ID NO: 74 - DNA, 114 bp, Artificial Sequence
  Synthetic Construct
  misc_feature 28, 58, 97: n = A,T,C or G
tgctttgtttgggatctgatggattaanggtcctggggttgacatctgtttggctctnggctgattgcgggatcgaacctctggtgcttggaacaanacatgttcttggcggaa

SEQ ID NO: 75 - DNA, 114 bp, Artificial Sequence
  Synthetic Construct
  misc_feature 96, 109: n = A,T,C or G
tgctttgtttgggatctgatggattaatggtcctgggattgacgtctgtttggttctggctgatgcagggatcgaacctctggtgcttggaacaanacatgttcttggnggaaa

SEQ ID NO: 76 - DNA, 95 bp, Artificial Sequence
  Synthetic Construct
gcctcagcaagctatcaaagtcggaacgctcaacgattgcccctcacgaggggaccagttgacaatccagccaatttcgataattccttaattaa

SEQ ID NO: 77 - DNA, 131 bp, Artificial Sequence
  Synthetic Construct
gcctcagcaagctatcaaagtcggaacgctcaacgattgcccctcacgaggggaccagttgacaatccagccaatgtcggaacgctcaacgattgcccctcacgaggggacttcgataattccttaattaa

SEQ ID NO: 78 - DNA, 243 bp, Artificial Sequence
  Synthetic Construct
gcctcagcaagctatcaaagtcggaacgctcaacgattgcccctcacgaggggaccagttgacaatccagccaatgtcggaacgctcaacgattgcccctcacgaggggaccagttgacaatccagccaatgtcggaacgctcaacgattgcccctcacgaggggaccagttgacaatccagccaatgtcggaacgctcaacgattgcccctcacgaggggacttcgataattccttaattaa

SEQ ID NO: 79 - DNA, 105 bp, Artificial Sequence
  Synthetic Construct
gcctcagcaagctatcaaagtcggaacgctcaacgattgcccctcacgaggggaccagttgacaatccagccaatcctgcaattattcgataattccttaattaa

SEQ ID NO: 80 - DNA, 273 bp, Artificial Sequence
  Synthetic Construct
gcctcagcaagctatcaaagtcggaacgctcaacgattgcccctcacgaggggaccagttgacaatccagccaatcctgcaattagtcggaacgctcaacgattgcccctcacgaggggaccagttgacaatccagccaatcctgcaattagtcggaacgctcaacgattgcccctcacgaggggaccagttgacaatccagccaatcctgcaattagtcggaacgctcaacgattgcccctcacgaggggacttcgataattccttaattaa

SEQ ID NO: 81 - DNA, 183 bp, Artificial Sequence
  Synthetic Construct
gcctcagctccgacctgatgagtccgtgaggacgaaacgagtaagctcgtcgtcggaacgctcaacgattgcccctcacgaggggaccagttgacaatccagccaatggccggcatggtcccagcctcctcgctggcgccggctgggcaacatgcttcggcatggcgaatgggacttaattaa

SEQ ID NO: 82 - DNA, 216 bp, Artificial Sequence
  Synthetic Construct
gcctcagcatcagagtggcgcagcggaagcgtggtgggcccataacccacaggtcccaggatcgaaacctggctctgatagtcggaacgctcaacgattgcccctcacgaggggaccagttgacaatccagccaatatcagagtggcgcagcggaagcgtggtgggcccataacccacaggtcccaggatcgaaacctggctctgatattaattaa

SEQ ID NO: 83 - DNA, 234 bp, Artificial Sequence
  Synthetic Construct
gcctcagcgagctctgaatcagagtggcgcagcggaagcgtggtgggcccataacccacaggtcccaggatcgaaacctggctctgatagtcggaacgctcaacgattgcccctcacgaggggaccagttgacaatccagccaatgagctctgaatcagagtggcgcagcggaagcgtggtgggcccataacccacaggtcccaggatcgaaacctggctctgatattaattaa

SEQ ID NO: 84 - DNA, 220 bp, Artificial Sequence
  Synthetic Construct
gcctcagcgctcccgtagctcagttggttagagcgttggtcttatgagccgaaggtcgcgggttcgagccccgccggaagcagtcggaacgctcaacgattgcccctcacgaggggaccagttgacaatccagccaatgctcccgtagctcagttggttagagcgttggtcttatgagccgaaggtcgcgggttcgagccccgccggaagcattaattaa

SEQ ID NO: 85 - DNA, 238 bp, Artificial Sequence
  Synthetic Construct
gcctcagcgagctctgagctcccgtagctcagttggttagagcgttggtcttatgagccgaaggtcgcgggttcgagccccgccggaagcagtcggaacgctcaacgattgcccctcacgaggggaccagttgacaatccagccaatgagctctgagctcccgtagctcagttggttagagcgttggtcttatgagccgaaggtcgcgggttcgagccccgccggaagcattaattaa

SEQ ID NO: 86 - DNA, 112 bp, Artificial Sequence
  Synthetic Construct
gcctcagcgttcactgccgtataggcaggtcggaacgctcaacgattgcccctcacgaggggaccagttgacaatccagccaatgttcactgccgtataggcagttaattaa

SEQ ID NO: 87 - DNA, 264 bp, Artificial Sequence
  Synthetic Construct
gcctcagcgttcactgccgtataggcaggtcggaacgctcaacgattgcccctcacgaggggaccagttgacaatccagccaatgttcactgccgtataggcaggtcggaacgctcaacgattgcccctcacgaggggaccagttgacaatccagccaatgttcactgccgtataggcaggtcggaacgctcaacgattgcccctcacgaggggaccagttgacaatccagccaatgttcactgccgtataggcagttaattaa

SEQ ID NO: 88 - protein, 9 aa, Artificial Sequence
  Synthetic Construct
SSGPPPGTG

SEQ ID NO: 89 - protein, 12 aa, Artificial Sequence
  Synthetic Construct
AEAAAKEAAAKA

SEQ ID NO: 90 - protein, 16 aa, Artificial Sequence
  Synthetic Construct
SGSETPGTSESATPES

SEQ ID NO: 91 - DNA, 36 bp, Artificial Sequence; identical to SEQ ID NO: 32
  Synthetic Construct

SEQ ID NO: 92 - DNA, 25 bp, Artificial Sequence
  Synthetic Construct
caacgattgcccctcacgaggggac

SEQ ID NO: 93 - DNA, 20 bp, Artificial Sequence
  Synthetic Construct
aaacgggtttttggaggcac

SEQ ID NO: 94 - DNA, 20 bp, Artificial Sequence
  Synthetic Construct
ctatgccaagtaaacctgga

SEQ ID NO: 95 - DNA, 20 bp, Artificial Sequence
  Synthetic Construct
tatgccaagtaaacctggag

SEQ ID NO: 96 - DNA, 20 bp, Artificial Sequence
  Synthetic Construct
aggcactttcatctggaggt

SEQ ID NO: 97 - DNA, 20 bp, Artificial Sequence
  Synthetic Construct
gccttatcaaaacgggtttt

SEQ ID NO: 98 - DNA, 20 bp, Artificial Sequence
  Synthetic Construct
ttgctatgccaagtaaacct

SEQ ID NO: 99 - DNA, 20 bp, Artificial Sequence
  Synthetic Construct
taggacatctgggaagtcaa

SEQ ID NO: 100 - DNA, 20 bp, Artificial Sequence
  Synthetic Construct
ttgttccgcaaaatagccca

SEQ ID NO: 101 - DNA, 20 bp, Artificial Sequence
  Synthetic Construct
aaagtacctggctgatgcag

SEQ ID NO: 102 - DNA, 20 bp, Artificial Sequence
  Synthetic Construct
cagttgacaatccagccaat

SEQ ID NO: 103 - DNA, 20 bp, Artificial Sequence
  Synthetic Construct
gcgacacgactcattataac

SEQ ID NO: 104 - RNA, 25 nt, Artificial Sequence
  Synthetic Construct
caacgauugccccucacgaggggac

SEQ ID NO: 105 - RNA, 20 nt, Artificial Sequence
  Synthetic Construct
ucccauucaacauucauacg

SEQ ID NO: 106 - RNA, 20 nt, Artificial Sequence
  Synthetic Construct
ucgaagcccauacaucuuuc

SEQ ID NO: 107 - RNA, 20 nt, Artificial Sequence
  Synthetic Construct
ugggccgaagcccauacauc

SEQ ID NO: 108 - RNA, 20 nt, Artificial Sequence
  Synthetic Construct
ugguucuauacuaauaucaa

SEQ ID NO: 109 - RNA, 20 nt, Artificial Sequence
  Synthetic Construct
auauuaguauagaaccauaa

SEQ ID NO: 110 - RNA, 20 nt, Artificial Sequence
  Synthetic Construct
guauagaaccauaacaaaag

SEQ ID NO: 111 - RNA, 20 nt, Artificial Sequence
  Synthetic Construct
cuaaauuuaguaaagaauca

SEQ ID NO: 112 - RNA, 20 nt, Artificial Sequence
  Synthetic Construct
guaaucaaugguuauuguga

SEQ ID NO: 113 - RNA, 20 nt, Artificial Sequence
  Synthetic Construct
ugaaaugaaauuuaacuuuu

SEQ ID NO: 114 - RNA, 20 nt, Artificial Sequence
  Synthetic Construct
guuaucuaaauaaaacuagg

SEQ ID NO: 115 - RNA, 20 nt, Artificial Sequence
  Synthetic Construct
gcgacacgacucauuauaac

(19) United States
(12) Patent Application Publication
(10) Pub. No.: US 2023/0159943 A1
(43) Pub. Date: May 25, 2023

(54) CRISPR SYSTEMS IN PLANTS

(71) Applicant: The Regents of the University of California, Oakland, CA (US)

(72) Inventors: Steve E. JACOBSEN, Agoura Hills, CA (US); Zheng LI, Los Angeles, CA (US); Jennifer DOUDNA, Berkeley, CA (US); Patrick PAUSH, Berkeley, CA (US); Basem AL-SHAYEB, Berkeley, CA (US)

(73) Assignee: The Regents of the University of California, Oakland, CA (US)

(21) Appl. No.: 17/919,503

(22) PCT Filed: Apr. 20, 2021
(86) PCT No.: PCT/US2021/028105

§ 371 Date: Oct. 17, 2022

Related U.S. Application Data
(60) Provisional application No. 63/146,468, filed on Feb. 5, 2021.
(60) Provisional application No. 63/012,634, filed on Apr. 20, 2020.

(51) Int. Cl. C12N 15/82; C12N 9/22

(52) U.S. Cl. CPC C12N 15/8213; C12N 9/22; C12N 2310/20

(57) ABSTRACT

The present disclosure relates to CRISPR-Cas systems that utilize Cas 12J for editing nucleic acids in plants. Methods and compositions for using these systems for editing nucleic acids in plants are provided herein.

### CROSS-REFERENCE TO RELATED APPLICATIONS

This application claims the benefit of U.S. Provisional Application No. 63/012,634, filed on Apr. 20, 2020, and U.S. Provisional Application No. 63/146,468, filed on Feb. 5, 2021, each of which is incorporated herein by reference in its entirety.

### STATEMENT REGARDING FEDERALLY-SPONSORED RESEARCH

This invention was made with government support under Grant Number AI142817, awarded by the National Institutes of Health. The government has certain rights in the invention.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 262232002240SEQLIST.TXT, date recorded: Apr. 19, 2021, size: 252 KB).

### FIELD

The present disclosure relates to CRISPR-Cas systems that utilize Cas12J for editing nucleic acids in plants. Methods and compositions for using these systems for editing nucleic acids in plants are provided herein.

### BACKGROUND

RNA-guided endonucleases (e.g. Cas polypeptide endonucleases that facilitate CRISPR-based nucleic acid editing) can be used as tools for genome editing. However, their versatility is limited by restrictions imposed by several requirements, including short recognition motifs referred to as protospacer-adjacent motifs (PAMs) and the fact that some RNA-guided nucleases either exhibit no functionality or greatly reduced functionality in eukaryotic organisms. In particular, there exists a need for improved CRISPR-Cas systems for targeting and editing nucleic acids in plants.

### BRIEF SUMMARY

In one aspect, the present disclosure provides a method for modifying a target nucleic acid in a plant cell, the method including: a) providing a plant cell including a recombinant Cas12J polypeptide and a guide RNA, and b) cultivating the plant cell under conditions whereby the Cas12J polypeptide and guide RNA are present as a complex that targets the target nucleic acid to generate a modification in the target nucleic acid. In some embodiments, the recombinant Cas12J polypeptide includes an amino acid sequence having at least 80% amino acid identity to SEQ ID NO: 2. In some embodiments that may be combined with any of the preceding embodiments, the recombinant Cas12J polypeptide includes a nuclear localization signal (NLS). In some embodiments, the nuclear localization signal is an SV40-type NLS. In some embodiments that may be combined with any of the preceding embodiments, the recombinant Cas12J polypeptide and guide RNA are encoded from one or more recombinant nucleic acids in the plant cell. In some embodiments, one of more of the recombinant nucleic acids include at least one intron. In some embodiments, one of more of the recombinant nucleic acids include a promoter that is functional in plants. In some embodiments, the promoter is a UBQ10 promoter. In some embodiments, the UBQ10 promoter includes a nucleic acid sequence that is at least 80% identical to SEQ ID NO: 23. In some embodiments that may be combined with any of the preceding embodiments, expression of the guide RNA is driven by an RNA Polymerase II promoter. In some embodiments, the RNA Polymerase II promoter is a CmYLCV promoter or a 2×35S promoter. In some embodiments, the promoter comprises a nucleic acid sequence that is at least 80% identical to SEQ ID NO: 29 or SEQ ID NO: 34. In some embodiments that may be combined with any of the preceding embodiments, the plant cell is cultivated at a temperature in the range of about 23° C. to about 37° C. In some embodiments that may be combined with any of the preceding embodiments, the plant cell is cultivated at a temperature in the range of about 20° C. to about 25° C. In some embodiments that may be combined with any of the preceding embodiments, the modification includes a deletion of one or more nucleotides in the target nucleic acid. In some embodiments that may be combined with any of the preceding embodiments, the deletion includes deletion of 3-15 nucleotides in the target nucleic acid. In some embodiments, the deletion includes deletion of 9 nucleotides in the target nucleic acid. In some embodiments that may be combined with any of the preceding embodiments, the target nucleic acid sequence is located in a region of repressive chromatin. In some embodiments that may be combined with any of the preceding embodiments, the target nucleic acid sequence is located in a region of open chromatin. In some embodiments that may be combined with any of the preceding embodiments, the guide RNA is recombinantly fused to a ribozyme. In some embodiments that may be combined with any of the preceding embodiments, the plant cell comprises a genetic background that exhibits reduced susceptibility to transgene silencing.

In another aspect, the present disclosure provides a recombinant vector including a nucleic acid sequence that includes a promoter that is functional in plants and that encodes a recombinant Cas12J polypeptide and a guide RNA. In some embodiments, the recombinant Cas12J polypeptide includes an amino acid sequence having at least 80% amino acid identity to SEQ ID NO: 2. In some embodiments that may be combined with any of the preceding embodiments, the recombinant Cas12J polypeptide includes a nuclear localization signal (NLS). In some embodiments, the nuclear localization signal is an SV40-type NLS. In some embodiments that may be combined with any of the preceding embodiments, the nucleic acid sequence includes at least one intron. In some embodiments, the promoter is a UBQ10 promoter. In some embodiments, the UBQ10 promoter includes a nucleic acid sequence that is at least 80% identical to SEQ ID NO: 23. In some embodiments that may be combined with any of the preceding embodiments, expression of the guide RNA is driven by an RNA Polymerase II promoter. In some embodiments, the RNA Polymerase II promoter is a CmYLCV promoter or a 2×35S promoter. In some embodiments, the promoter comprises a nucleic acid sequence that is at least 80% identical to SEQ ID NO: 29 or SEQ ID NO: 34. In some embodiments that may be combined with any of the preceding embodiments, the guide RNA is recombinantly fused to a ribozyme.

In another aspect, the present disclosure provides a plant cell including a recombinant Cas12J polypeptide and a guide RNA, wherein the Cas12J polypeptide and guide RNA are capable of existing in a complex that targets a target nucleic acid to generate a modification in the target nucleic acid. In some embodiments, the recombinant Cas12J polypeptide includes an amino acid sequence having at least 80% amino acid identity to SEQ ID NO: 2. In some embodiments that may be combined with any of the preceding embodiments, the recombinant Cas12J polypeptide includes a nuclear localization signal (NLS). In some embodiments, the nuclear localization signal is an SV40-type NLS. In some embodiments that may be combined with any of the preceding embodiments, the recombinant Cas12J polypeptide and guide RNA are encoded from one or more recombinant nucleic acids in the plant cell. In some embodiments, one of more of the recombinant nucleic acids include at least one intron. In some embodiments, one of more of the recombinant nucleic acids include a promoter that is functional in plants. In some embodiments, the promoter is a UBQ10 promoter. In some embodiments, the UBQ10 promoter includes a nucleic acid sequence that is at least 80% identical to SEQ ID NO: 23. In some embodiments that may be combined with any of the preceding embodiments, expression of the guide RNA is driven by an RNA Polymerase II promoter. In some embodiments, the RNA Polymerase II promoter is a CmYLCV promoter or a 2×35S promoter. In some embodiments, the promoter comprises a nucleic acid sequence that is at least 80% identical to SEQ ID NO: 29 or SEQ ID NO: 34. In some embodiments that may be combined with any of the preceding embodiments, the plant cell is cultivated at a temperature in the range of about 23° C. to about 37° C. In some embodiments that may be combined with any of the preceding embodiments, the plant cell is cultivated at a temperature in the range of about 20° C. to about 25° C. In some embodiments that may be combined with any of the preceding embodiments, the modification includes a deletion of one or more nucleotides in the target nucleic acid. In some embodiments that may be combined with any of the preceding embodiments, the deletion includes deletion of 3-15 nucleotides in the target nucleic acid. In some embodiments, the deletion includes deletion of 9 nucleotides in the target nucleic acid. In some embodiments that may be combined with any of the preceding embodiments, the target nucleic acid sequence is located in a region of repressive chromatin. In some embodiments that may be combined with any of the preceding embodiments, the target nucleic acid sequence is located in a region of open chromatin. In some embodiments that may be combined with any of the preceding embodiments, the guide RNA is recombinantly fused to a ribozyme. In some embodiments that may be combined with any of the preceding embodiments, the plant cell comprises a genetic background that exhibits reduced susceptibility to transgene silencing.

In another aspect, the present disclosure provides a plant including a plant cell of any one of the preceding embodiments, wherein the plant includes a modified nucleic acid. In some embodiments, the modification includes a deletion of one or more nucleotides in the nucleic acid. In some embodiments that may be combined with any of the preceding embodiments, the deletion includes deletion of 3-15 nucleotides. In some embodiments, the deletion includes deletion of 9 nucleotides.

In another aspect, the present disclosure provides a progeny plant of the plant of any one of the preceding embodiments, wherein the progeny plant includes a modified nucleic acid. In some embodiments, the modification includes a deletion of one or more nucleotides in the nucleic acid. In some embodiments that may be combined with any of the preceding embodiments, the deletion includes deletion of 3-15 nucleotides. In some embodiments, the deletion includes deletion of 9 nucleotides.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 illustrates a diagram of the AtPDS3 gene and the locations of AtPDS3 gRNA1 to gRNA10.

FIG. 2 illustrates that RNPs of CAS12J-2 protein and AtPDS3 gRNA are able to cleave AtPDS3 PCR fragment in vitro at 37° C. AtPDS3 gene fragments spanning all gRNA target regions were amplified by PCR and gel purified. The size of uncleaved fragments is 2.76 kb. AtPDS3 gene fragments were incubated with CAS12J-2 RNPs with gRNA1 to gRNA10, as well as a scrambled gRNA control at 37° C. for 1 hour. Reactions were stopped by addition of EDTA and digestion of CAS12J-2 protein with proteinase K. A 2% agarose gel was used to visualize the cleavage products. DNA ladders are shown in the far left and far right lanes, with size labels flanking. The lane labeled gR1 shows the reaction products when incubated with RNP-gRNA1. The lane labeled gR2 shows the reaction products when incubated with RNP-gRNA2. The lane labeled gR3 shows the reaction products when incubated with RNP-gRNA3. The lane labeled gR4 shows the reaction products when incubated with RNP-gRNA4. The lane labeled gR5 shows the reaction products when incubated with RNP-gRNA5. The lane labeled gR6 shows the reaction products when incubated with RNP-gRNA6. The lane labeled gR7 shows the reaction products when incubated with RNP-gRNA7. The lane labeled gR8 shows the reaction products when incubated with RNP-gRNA8. The lane labeled gR9 shows the reaction products when incubated with RNP-gRNA9. The lane labeled gR10 shows the reaction products when incubated with RNP-gRNA10. The lane labeled Scramble shows the reaction products when incubated with the RNP-scrambled gRNA control.

FIG. 3 illustrates a Western blot of flag-tagged CAS12J-2 protein. The lane labeled “M” includes a protein ladder, with corresponding weights labeled along the left side. The lane labeled “1-1” includes a protoplast sample transformed with no plasmid. The lane labeled “1-2” includes a protoplast sample transformed with HBT-sGFP (S65T) plasmid as control. The lane labeled “1-3” includes a protoplast sample transformed with pCAMBIA1300_pUB10_pcoCAS12J2_E9t_version1 AtPDS3 guide 1. The lane labeled “1-4” includes a protoplast sample transformed with pCAMBIA1300_pUB10_pcoCAS12J2_E9t_version1 AtPDS3 guide 2. The lane labeled “1-5” includes a protoplast sample transformed with pCAMBIA1300_pUB10.pcoCAS12J2_E9t_version2 AtPDS3 guide 1. The lane labeled “1-6” includes a protoplast sample transformed with pCAMBIA1300_pUB10_.pcoCAS12J2_E9t_version2 AtPDS3 guide 2. Protoplasts were incubated at 23° C. for 48 h.

FIG. 4 illustrates a summary of amplicon sequencing results, and shows the percentage of reads with deletions. Results shown are from Arabidopsis protoplasts transfected with pCAMBIA1300_pUB10_pcoCAS12J2_E9t_version1 AtPDS3 guide (guide 1 to guide 5) plasmid (ver1), or pCAMBIA1300_pUB10_pcoCAS12J2_E9t_version2 AtPDS3 guide (guide 1 to guide 5) plasmid (ver2), or RNPs of CAS12J-2 with AtPDS3 guide1 to guide 10 (RNP) as well as control samples amplified for the same regions of interest. Percent of reads with deletions among all reads spanning the region of interest are plotted. Regions labeled “23 C” indicate that protoplast samples were incubated at 23° C. after transfection. Regions labeled “37 C” indicate that protoplast samples were incubated at 23° C. with a 37° C. heat shock incubation applied in the middle of the incubation period. The percentage of reads with deletions is plotted for each condition. The criteria to classify reads as reads with deletion were as follows: only reads with >=3 bp deletions of the same pattern (deletion of the same size starting with the same location) with >=100 reads counts from a sample are counted into reads number with deletion. These criteria were established by assessing read patterns and corresponding reads counts in all control samples to avoid counting PCR errors or sequencing errors as true signals.

FIG. 5A-FIG. 5F illustrate the frequency of reads with deletions, summarized for each size of deletion, for gRNA5, gRNA8 and gRNA10. FIG. 5A shows results for gRNA5 targeting. 6 samples that showed editing in gRNA5-targeted region were combined for analysis. FIG. 5B shows all 4 control samples for gRNA5 combined for analysis. FIG. 5C shows results for gRNA8 targeting. 2 samples that showed editing in gRNA8-targeted region were combined for analysis. FIG. 5D summarizes results from the only control sample for gRNA8. FIG. 5E shows results for gRNA10 targeting. 2 samples which showed editing in gRNA10-targeted region were combined for analysis. FIG. 5F shows the only control sample for gRNA10. For each of FIG. 5A-FIG. 5F, only read patterns with read counts of more than 100 were included in quantification. Reads with deletion size of 1 bp and 2 bp, as well as insertion size of 1 bp, were included in these graphs to show the background level of mutations that were also present in control samples.

FIG. 6A-FIG. 6B illustrate plasmid maps. FIG. 6A illustrates the map of pCAMBIA1300_pUB10_.pcoCAS12J2_E9t_version1_AtPDS3_gRNA1. FIG. 6B illustrates the map of pCAMBIA1300_pUB10_pcoCAS12J2_E9t_version2_AtPDS3_gRNA1.

FIG. 7 illustrates that RNPs of CAS12J-2 protein and AtPDS3 gRNA are able to cleave AtPDS3 PCR fragment in vitro at 23° C. An AtPDS3 gene fragment spanning all gRNA target regions was amplified by PCR and gel purified. The uncleaved fragment size is 2.76 kb. AtPDS3 gene fragments were incubated with CAS12J-2 RNPs with gRNA1 to gRNA10, as well as a scrambled gRNA control at 23° C. for 2 hours. Reactions were stopped by addition of EDTA and digestion of CAS12J-2 with proteinase K. A 1% agarose gel was used to visualize the cleavage products. DNA ladders are shown in the far left and far right lanes, with size labels flanking. The lane labeled gR1 shows the reaction products when incubated with RNP-gRNA1. The lane labeled gR2 shows the reaction products when incubated with RNP-gRNA2. The lane labeled gR3 shows the reaction products when incubated with RNP-gRNA3. The lane labeled gR4 shows the reaction products when incubated with RNP-gRNA4. The lane labeled gR5 shows the reaction products when incubated with RNP-gRNA5. The lane labeled gR6 shows the reaction products when incubated with RNP-gRNA6. The lane labeled gR7 shows the reaction products when incubated with RNP-gRNA7. The lane labeled gR8 shows the reaction products when incubated with RNP-gRNA8. The lane labeled gR9 shows the reaction products when incubated with RNP-gRNA9. The lane labeled gR10 shows the reaction products when incubated with RNP-gRNA10. The lane labeled Scramble shows the reaction products when incubated with the scrambled RNP-gRNA control.

FIG. 8 illustrates a summary of the amplicon sequencing results, showing the percentage of reads with deletions in Arabidopsis protoplasts transfected with pCAMBIA1300_pUB10_pcoCAS12J2_E9t_version1 AtPDS3 guide (guide5, guide8 or guide 10) plasmids (ver1), or pCAMBIA1300_pUB10_pcoCAS12J2_E9t_version2 AtPDS3 guide (guide5, guide8 or guide 10) plasmids (ver2), or RNPs of CAS12J-2 with AtPDS3 guide5, guide8 or guide 10 (RNP) as well as GFP control samples amplified for the same regions of interest. The percentage of reads with deletions among all reads spanning the region of interest is plotted. Regions labeled “23 C” indicate that protoplast samples were incubated at 23° C. after transfection. Regions labeled “37 C” indicate that protoplast samples were incubated at 23° C. with a 37° C. heat shock incubation applied in the middle of the incubation at 23° C.

FIG. 9A-FIG. 9F illustrate the frequency of reads with deletions for each size of deletion for gRNA5, gRNA8 and gRNA10. FIG. 9A depicts the results for gRNA5, for which 6 editing samples that showed editing in gRNA5-targeted region were combined for analysis. FIG. 9B summarizes results from a control sample for gRNA5. FIG. 9C depicts the results for gRNA8, for which 6 editing samples that showed editing in gRNA8-targeted region were combined for analysis. FIG. 9D summarizes results from a control sample for gRNA8. FIG. 9E depicts the results for gRNA10, for which 6 editing samples that showed editing in gRNA10-targeted region were combined for analysis. FIG. 9F summarizes 2 control samples for gRNA10. For each of FIG. 9A-FIG. 9F, only read patterns with read counts more than 100 were included in quantification. Reads with deletion sizes of 1 bp and 2 bp, as well as insertion size of 1 bp, were included in these graphs to show the background level of mutations that were also present in control samples.

FIG. 10 illustrates that protoplast transfection efficiency was significantly decreased by spiking in CB buffer. In RNP transfection experiments, the 2×CB buffer in which RNPs were reconstituted was also added to transfection reaction. To determine if the composition of CB buffer affected transfection efficiency, 10 μg of HBT-sGFP (S65T) plasmid was transfected into 4×10⁴ protoplasts without CB buffer (top row) or with addition of CB buffer (13 μl of 2×CB buffer; pictures in bottom row). Pictures were taken after 10 hours of 23° C. incubation following transfection. Cells with GFP signal were counted in the GFP picture and the total number of intact cells (unfractured) was counted in the brightfield pictures. Cell numbers and transfection efficiency are summarized in Table 3-1.

FIG. 11A-FIG. 11B illustrate plasmid maps. FIG. 11A illustrates the map of pCAMBIA1300_pYAO_pcoCAS12J2_version1_AtPDS3_gRNA10. FIG. 11B illustrates the map of pCAMBIA1300_pYAO_pcoCAS12J2_version2_AtPDS3_gRNA10.

FIG. 12A-FIG. 12B illustrate that a T1 plant selected from transformation of pCAMBIA1300 pUB10 pcoCAS12J2 E9t version1 AtPDS3 gR10 plasmid is mosaic for heterozygous mutation in the AtPDS3 gR10 target region. FIG. 12A illustrates that initial sanger sequencing showed that one leaf of T1 transgenic plant number 33 was heterozygous for mutation in the AtPDS3 gR10 target region. Sequences from top to bottom are SEQ ID NO: 45-48. FIG. 12B illustrates that amplicon sequencing of DNA extracted from different parts of T1 plant 33 showed that it is mosaic for the mutation.

FIG. 13A-FIG. 13C illustrate CAS12J-2-mediated editing detected by amplicon sequencing in multiple CAS12J-2 T1 transgenic plants. FIG. 13A illustrates that a low frequency of editing was detected with amplicon sequencing in CAS12J-2 T1 transgenic plant number 4 with AtPDS3 gR5. T1 plant 4, 5 and 9 were screened from pCAMBIA1300 pUB10 pcoCAS12J2 E9t version 1 AtPDS3 gR5 transformation. T1 plant 11 was screened from pCAMBIA1300 pUB10 pcoCAS12J2 E9t version 2 AtPDS3 gR5 transformation. FIG. 13B illustrates that a low frequency of editing was detected with amplicon sequencing in CAS12J-2 T1 transgenic plants with AtPDS3 gR8. T1 plant 8 and 12 were screened from a pCAMBIA1300 pUB10 pcoCAS12J2 E9t version 1 AtPDS3 gR8 transformation, while T1 plant 3 and 4 were screened from a pCAMBIA1300 pUB10 pcoCAS12J2 E9t version 2 AtPDS3 gR8 transformation. FIG. 13C illustrates that editing was detected with amplicon sequencing in CAS12J-2 T1 transgenic plants with AtPDS3 gR10. T1 plant 1-6 were screened at 28° C. from a pCAMBIA1300 pUB10 pcoCAS12J2 E9t version 2 AtPDS3 gR10 transformation, while the other T1 plants in (C) were screened at room temperature from a pCAMBIA1300 pUB10 pcoCAS12J2 E9t version 1 AtPDS3 gR10 transformation.

FIG. 14A-FIG. 14E illustrate homozygous mutations of the AtPDS3 gene that were identified from offspring of seedlings of pCAMBIA1300 pUB10 pcoCAS12J2 E9t version1 AtPDS3 gR10 T1 plant 33. FIG. 14A illustrates an earlier batch of T2 seeds harvested from T1 plant 33 that were grown on ½ MS medium plate. White circles mark the position of albino/dwarf seedlings. FIG. 14B illustrates a later batch of T2 seeds harvested from T1 plant 33 that were grown on ½ MS medium plate. White circles mark the position of albino/dwarf seedlings. FIG. 14C illustrates Sanger sequencing results (6 examples) of albino seedlings from T1 plant 33 offspring seedlings that were aligned to the wild type AtPDS3 gene sequence. Sequences from top to bottom are SEQ ID NO: 49-56. FIG. 14D illustrates AtPDS3 homolog protein sequences from different species that were aligned with Clustal Omega by the Geneious software. Sequences from top to bottom are SEQ ID NO: 57-67. FIG. 14E illustrates PCR amplification results for a fragment of the CAS12J-2 transgene from albino T2 seedling DNA. Seedling number is as indicated.

FIG. 15A-FIG. 15B illustrate additional CAS12J-2 editing examples identified in T2 seedlings. FIG. 15A illustrates Sanger sequencing results of the PCR amplified AtPDS3 target region from six T2 seedlings from pCAMBIA1300 pUB10 pcoCAS12J2 E9t version2 AtPDS3 gR10 T1 plant 6, showing that they are heterozygous for mutation in this region. Sequences from top to bottom are SEQ ID NO: 68-75. FIG. 15B illustrates T2 plants from pCAMBIA1300 pUB10 pcoCAS12J2 E9t version1 AtPDS3 gR10 T1 plant 33 (left) and pCAMBIA1300 pUB10 pcoCAS12J2 E9t version 2 AtPDS3 gR10 T1 plant 6 (right), which are heterozygous for mutation of the AtPDS3 gR10 target region and that showed white albino sectors on the leaves (arrows).

FIG. 16 illustrates locations of CAS12J-2 gRNAs targeting the promoter region of the FWA gene. The FWA gene (AT4G25530) position is indicated in the bottom track, with transcription start site (TSS) indicated (only part of the FWA gene is shown). Positions of CAS12J guide RNAs targeting the FWA promoter regions are indicated in the FWA gRNAs track. DNA methylation patch in WT plants (Col-0 ecotype) is shown in the DNA methylation track (including DNA methylation in CG, CHG and CHH contexts).

FIG. 17 illustrates that RNPs of CAS12J-2 protein and gRNAs targeting the FWA gene promoter are able to cleave an FWA promoter PCR fragment in vitro at 37° C. A 1.57 kb FWA gene fragment spanning all gRNA target regions was amplified by PCR and gel purified. The FWA gene fragment was incubated with CAS12J-2 RNPs containing gRNA1 to gRNA10 and a scrambled gRNA control at 37° C. for 1 hour. Reactions were stopped by adding EDTA and digestion of CAS12J-2 protein with proteinase K. 2% agarose gels were used to visualize the cleavage products along with a DNA ladder for sizing.

FIG. 18A illustrates amplicon sequencing results of Arabidopsis protoplasts transfected with RNPs of CAS12J-2 protein with FWA gRNAs. WT protoplasts results are on the top, and fwa-4 epiallele protoplast results are on the bottom. Percent of reads with deletions among all reads spanning the region of interest was plotted. RT: protoplast sample incubated at room temperature (RT, 23° C.) after transfection. 37° C.: protoplast sample incubated at 23° C. with a 37° C. incubation applied in the middle of the incubation. Percentage of reads with deletions is plotted for each condition. The criteria to classify reads as containing deletions: only reads with >=3 bp deletion of the same pattern (deletion of same size starting at the same location) with >=100 read counts from a sample were classified as reads with deletions. Specifically for FWA gRNA6 and gRNA9 targeted regions, there are long stretches of adenines starting from a few nucleotides after the gRNA target site ends. Due to the high error rate of polymerases in replicating long stretch of adenines, reads with deletions only within these stretches of adenines were not counted as true reads with deletions. This criteria is established by assessing reads patterns and corresponding reads counts in all control samples, so that PCR errors or sequencing errors will not be counted as true signal.

FIG. 18B illustrates that CAS12J-2 RNPs targeting DNA-methylated region of FWA promoter exhibited higher editing efficiency when transfected into fwa-4 epi-mutant protoplasts than WT protoplasts. Col-0 (WT) and fwa-4 epi-mutant plants were grown under the same condition and the protoplasts from both were prepared in parallel. CAS12J-2 RNPs with FWA gRNA1, gRNA4, gRNA5 and gRNA6 were transfected into prepared WT and fwa-4 protoplasts at the same time. Two replicate transfections were performed for each gRNA-protoplast combination. Mean editing efficiency and standard deviation of these two replicates were plotted. t test were used to calculate P value for each comparison. *, 0.01<P<0.05, **0.001<P<0.01.

FIG. 19A-FIG. 19C illustrate plasmid maps with gRNA cassettes driven by RNA Pol II promoters. FIG. 19A illustrates a map of pCAMBIA1300 pUB10 pcoCAS12J2 E9t ver2 CmYLCVp AtPDS3 gRNA10 35St. FIG. 19B illustrates a map of pCAMBIA1300 pUB10 pcoCAS12J2 E9t ver2 2×35Sp AtPDS3 gRNA10 HSP18t. FIG. 19C illustrates a map of pCAMBIA1300 pUB10 pcoCAS12J2 E9t ver2 insulator pUB10 AtPDS3 gRNA10 E9t.

FIG. 20 illustrates maps of three gRNA configurations tested with Pol II promoter-terminator combinations. Shown are: a single CAS12J-2 repeat followed by AtPDS3 gRNA10 (top); a CAS12J-2 repeat followed by AtPDS3 gRNA10 with another CAS12J-2 repeat at the end (middle); and a triple array of CAS12J-2 repeat-AtPDS3 gRNA10 followed by another CAS12J-2 repeat at the end (bottom). Sequences from top to bottom are SEQ ID NO: 76-78.

FIG. 21A-FIG. 21D illustrates that Pol II promoters are able to drive CAS12J-2 gRNA expression and cause editing in protoplasts. Three combinations of Pol II promoters and terminators were used to express CAS12J-2 gRNAs: CmYLCV promoter+35S terminator, 2×35S promoter+HSP18.2 terminator and UBQ10 promoter+RbcS-E9 terminator. Three configurations of gRNAs were also tested: a single AtPDS3 gR10 without end repeat, a single AtPDS3 gR10 with end repeat, and a triple AtPDS3 gR10 array with end repeat. FIG. 21A, FIG. 21B, and FIG. 21C illustrate summaries of editing efficiency at the target region (AtPDS3 gRNA10) in protoplasts in three different experiments, comparing promoter terminator combinations and gRNA configurations, with the original Pol III promoter AtU6-26 driving gR10 as a control. FIG. 21D illustrates the AtPDS3 gRNA10 expression level measured by quantitative PCR normalized to the housekeeping IPP2 gene in protoplasts transfected with the same amount of plasmids.

FIG. 22A-FIG. 22B illustrates that CAS12J-2 editing efficiency was not increased by AtPDS3 gRNA10 with 30 bp spacer. FIG. 22A illustrates maps of single AtPDS3 gRNA10 and triple AtPDS3 gRNA10 array with 30 bp spacer. Sequences from top to bottom are SEQ ID NO: 79-80. FIG. 22B illustrates CmYLCVp single gR10: CmYLCVp driving the expression of a single AtPDS3 gRNA10 with 20 bp spacer or 30 bp spacer without another CAS12J-2 CRISPR repeat at the end. CmYLCVp triple gR10, 2×35Sp triple gR10 and pUB10 triple gR10: Three Pol II promoter-terminator combination sets driving the expression of the triple AtPDS3 gRNA10 array with 20 bp spacer or 30 bp spacer. Mean editing efficiency and standard deviation of two replicates were plotted. t test were used to calculate P value for each comparison: *, 0.01<P<0.05, **0.001<P<0.01.

FIG. 23A-FIG. 23B illustrates that ribozyme mediated processing of gRNA increased CAS12J-2 editing efficiency. FIG. 23A illustrates a map of ribozymes flanking CAS12J-2 AtPDS3 gRNA10 (SEQ ID NO: 81): Hammerhead ribozyme stem loop is on the 5′ end of the CAS12J-2 AtPDS3 gRNA10 sequence and HDV ribozyme stem loop is on the 3′ end. There is a 6 base pair sequence before the Hammerhead ribozyme which is complementary to the beginning of CAS12J-2 CRISPR repeat for proper processing by ribozyme. FIG. 23B illustrates that for each Pol II promoter-terminator combination, the editing efficiency of a single CAS12J-2 AtPDS3 gR10 without extra repeat on the end was compared to that of a single CAS12J-2 AtPDS3 gR10 flanked by ribozymes. Mean editing efficiency and standard deviation of two replicates were plotted. t test were used to calculate P value for each comparison. *, 0.01<P<0.05.

FIG. 24 illustrates maps of single AtPDS3 gRNA10 flanked by tRNA^{Met}, long-tRNA^{Met}, tRNA^{Ile} and long-tRNA^{Ile}. Sequences from top to bottom are SEQ ID NO: 82-85.

FIG. 25 illustrates that target gene editing efficiency by CAS12J-2 was not increased by tRNA processing systems. For each Pol II promoter-terminator combination, CAS12J-2 editing efficiencies of single AtPDS3 gRNA10 without additional processing machinery or flanked by tRNAMet, long-tRNAMet, tRNAIle and long-tRNAIle were compared. Mean editing efficiency and standard deviation of two replicates were plotted. Within each promoter-terminator combination set, one way ANOVA followed by Dunnett's multiple comparison test were used to analyze if the difference between mean values of no processing machinery and with tRNA processing system reached significance. *, 0.01<P<0.05, **, 0.001<P<0.01, ****, P<0.0001.

FIG. 26A-FIG. 26B illustrate that target gene editing efficiency by CAS12J-2 was not increased by Csy4 gRNA processing system. FIG. 26A illustrates maps of single AtPDS3 gRNA10 and triple AtPDS3 gRNA10 array with Csy4 binding sites. Sequences from top to bottom are SEQ ID NO: 86-87. FIG. 26B illustrates that for each Pol II promoter-terminator combination and for single AtPDS3 gRNA10 and triple AtPDS3 gRNA10, CAS12J-2 editing efficiencies of gRNA expression cassettes with and without Csy4 gRNA processing systems were compared. Mean editing efficiency and standard deviation of two replicates were plotted. t test were used to calculate P value for each comparison. *, 0.01<P<0.05, **0.001<P<0.01.

FIG. 27 illustrates that RDR6 mediated transgene silencing negatively influenced editing efficiency in CAS12J-2 transgenic plants. pCAMBIA1300 pUB10 pcoCAS12J2 E9t version1 AtPDS3 gRNA 10 (version1) and pCAMBIA1300 pUB10 pcoCAS12J2 E9t version2 AtPDS3 gRNA 10 (version2) plasmids were used to generate transgenic plants in Col-0 (WT) and rdr6-15 backgrounds. 10 genotyped T1 plants were randomly selected for each category for amplicon sequencing and the editing efficiencies were plotted for each T1 plant ranked within each set. For the set of version 2 plasmid in rdr6-15 background, only 9 T1 plants were obtained. Wilcoxon matched-pairs signed rank test were used to calculate P value for each comparison indicated (WT vs rdr6-15 backgrounds for each plasmid). **, 0.01<P<0.05.

### DETAILED DESCRIPTION

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)). Harlow and Lane, eds. (1988); Oligonucleotide Synthesis (M. J. Gait, ed., 1984); Methods in Molecular Biology. Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Gene Transfer Vectors for Mammalian Cells (J. M. Miller and M. P. Calos, eds., 1987); PCR: The Polymerase Chain Reaction, (Mullis et al., eds., 1994); Short Protocols in Molecular Biology (Wiley and Sons, 1999).

### General Terms

The terminology used herein is for the purpose of describing particular embodiments and is not intended to be limiting.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “isolated” and “purified” as used herein refers to a material that is removed from at least one component with which it is naturally associated (e.g., removed from its original environment). The term “isolated,” when used in reference to an isolated protein, refers to a protein that has been removed from the culture medium of the host cell that expressed the protein. As such an isolated protein is free of extraneous or unwanted compounds (e.g., nucleic acids, native bacterial or other proteins, etc.).

It is understood that aspects and embodiments of the present disclosure described herein include “comprising,” “consisting,” and “consisting essentially of” aspects and embodiments.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### Overview

The following description is presented to enable a person of ordinary skill in the art to make and use the various embodiments. Descriptions of specific devices, techniques, methods, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

The present disclosure relates to CRISPR-Cas systems that utilize Cas12J for editing nucleic acids in plants. Methods and compositions for using these systems for editing nucleic acids in plants are provided herein.

In particular. Applicant has developed CRISPR systems utilizing Cas12J which are particularly well-suited for use in plants. Applicant's CRISPR-Cas12J systems work well at a wide variety of temperature ranges (e.g. 23° C. and 37° C.), with the room temperature ranges overlapping with the ideal temperatures for the growth of many plants, cold-blooded animals, and other organisms that live at lower temperatures. Thus, in addition to plants, CRISPR-targeting systems which use Cas12J may also be useful in cold blooded animals and other organisms that live at lower temperatures.

In general, a Cas12J polypeptide of the present disclosure is capable of forming a ribonucleoprotein (RNP) complex by binding to or otherwise interacting with a guide RNA (gRNA). The Cas12J-gRNA ribonucleoprotein complex is capable of being targeted to a target nucleic acid via base pairing between the guide RNA and a target nucleotide sequence in the target nucleic acid that is complimentary to the sequence of the guide RNA. The guide RNA thus provides the specificity for targeting a particular target nucleic. Once the Cas12J-gRNA ribonucleoprotein complex has come into association with a target nucleic acid by virtue of the targeting of the RNP complex to that target nucleic acid by the guide RNA, the Cas12J protein is able to have activity at that target nucleic acid and accordingly edit the target nucleic acid.

Accordingly, the present disclosure provides RNA-guided CRISPR-Cas effector polypeptides for use in CRISPR-based targeting systems in plants. In particular, the present disclosure provides Cas12J polypeptides, sometimes also referred to as Case or CasXS polypeptides, for use in CRISPR-based targeting systems in plants. Provided herein are Cas12J polypeptides, nucleic acids encoding the same, compositions containing the same, and methods of using the same to e.g. edit a target nucleic acid. The present disclosure provides ribonucleoprotein complexes containing a Cas12J polypeptide and a guide RNA which may be used to e.g. edit a target nucleic acid. The present disclosure provides methods of modifying a target nucleic acid in plants using a Cas12J polypeptide and a guide RNA. The present disclosure also provides guide RNAs that bind to and provide target sequence specificity to Cas12J polypeptides. Provided herein are guide RNAs that can bind or otherwise interact with Cas12J polypeptides, nucleic acids encoding the same, compositions containing the same, and methods of using the same to e.g. edit a target nucleic acid.

### Recombinant Polypeptides

Certain aspects of the present disclosure relate to recombinant polypeptides (e.g. Cas12J polypeptides) and their use in CRISPR-based targeting systems in e.g. plants.

As used herein, a “polypeptide” is an amino acid sequence including a plurality of consecutive polymerized amino acid residues (e.g., at least about 15 consecutive polymerized amino acid residues). “Polypeptide” refers to an amino acid sequence, oligopeptide, peptide, protein, or portions thereof, and the terms “polypeptide” and “protein” are used interchangeably.

Polypeptides as described herein also include polypeptides having various amino acid additions, deletions, or substitutions relative to the native amino acid sequence of a polypeptide of the present disclosure. In some embodiments, polypeptides that are homologs of a polypeptide of the present disclosure contain non-conservative changes of certain amino acids relative to the native sequence of a polypeptide of the present disclosure. In some embodiments, polypeptides that are homologs of a polypeptide of the present disclosure contain conservative changes of certain amino acids relative to the native sequence of a polypeptide of the present disclosure, and thus may be referred to as conservatively modified variants. A conservatively modified variant may include individual substitutions, deletions or additions to a polypeptide sequence which result in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). A modification of an amino acid to produce a chemically similar amino acid may be referred to as an analogous amino acid.

A “recombinant” polypeptide, protein, or enzyme of the present disclosure is a polypeptide, protein, or enzyme that may be encoded by e.g. a “recombinant nucleic acid” or “heterologous nucleic acid” or “recombinant polynucleotide.”

Recombinant polypeptides of the present disclosure that are composed of individual polypeptide domains may be described based on the individual polypeptide domains of the overall recombinant polypeptide. A domain in such a recombinant polypeptide refers to the particular stretches of contiguous amino acid sequences with a particular function or activity. For example, a recombinant polypeptide that is a fusion of a Cas12J polypeptide and an additional polypeptide providing further function or activity, the contiguous amino acids that encode the Cas12J polypeptide may be described as the Cas12J domain in the overall recombinant polypeptide. Individual domains in an overall recombinant protein may also be referred to as units of the recombinant protein. Recombinant polypeptides that are composed of individual polypeptide domains may also be referred to as fusion polypeptides.

Polypeptides of the present disclosure may be detecting using antibodies. Techniques for detecting polypeptides using antibodies include, for example, enzyme linked immunosorbent assays (ELISAs), Western blots, immunoprecipitations, and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

Cas12J Polypeptides

Certain aspects of the present disclosure relate to Cas12J polypeptides and their use in facilitating the editing/modification of a target nucleic acid. Cas12J polypeptides generally function as RNA-guided DNA-binding proteins. Cas12.1 polypeptides may have endonuclease activity which can facilitate modification/editing of a target nucleic acid.

Various Cas12J polypeptides may be used in the methods and compositions of the present disclosure, including full-length Cas12J proteins and fragments thereof. In some embodiments, a Cas12J polypeptide contains at least 20 consecutive amino acids, at least 30 consecutive amino acids, at least 40 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 120 consecutive amino acids, at least 140 consecutive amino acids, at least 160 consecutive amino acids, at least 180 consecutive amino acids, at least 200 consecutive amino acids, at least 220 consecutive amino acids, at least 240 consecutive amino acids, at least 260 consecutive amino acids, at least 280 consecutive amino acids, at least 300 consecutive amino acids, at least 350 consecutive amino acids, at least 400 consecutive amino acids, at least 450 consecutive amino acids, at least 500 consecutive amino acids, at least 550 consecutive amino acids, at least 600 consecutive amino acids, at least 650 consecutive amino acids, or at least 750 consecutive amino acids or more of a full-length Cas12J protein. In some embodiments, a Cas122J polypeptide may include sequences with one or more amino acids removed from the consecutive amino acid sequence of a full-length Cas12J protein. In some embodiments, a Cas12J polypeptide may include sequences with one or more amino acids replaced/substituted with an amino acid different from the endogenous amino acid present at a given amino acid position in a consecutive amino acid sequence of a full-length Cas12J protein. In some embodiments, a Cas12J polypeptide may include sequences with one or more amino acids added to an otherwise consecutive amino acid sequence of a full-length Cas12J protein.

Examples of Cas12J proteins are provided in SEQ ID NO: 1-10. In some embodiments, a Cas12J polypeptide of the present disclosure has an amino acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of any one of SEQ ID NO: 1, 2, 3, 4, 5, 6, 7, 8, 9, and/or 10.

One of skill in the art would recognize additional Cas12J proteins or fragments thereof, homologs thereof, and/or orthologs thereof that may be used herein. For example, Cas12J proteins are described in Al-Shayeb et al., “Clades of huge phages from across Earth's ecosystems,” Nature, Volume 578.

Cas12J polypeptides of the present disclosure may contain a number of modifications to alter their activity and/or function as will be readily apparent to one of skill in the art. For example, a Cas12J polypeptide may be modified to be nuclease deficient (also referred to as “dCas12J polypeptides”) such that they are no longer capable of cleaving or otherwise introducing strand breaks in a target nucleic acid molecule. Cas12J polypeptides of the present disclosure may also be modified to include additional polypeptide domains that confer additional function. For example, a dCas12J polypeptide could be recombinantly fused to e.g. a DNA methyltransferase polypeptide for use in a system to confer targeted DNA methylation of a target nucleic acid. Exemplary DNA methyltransferase polypeptides or domains thereof that could be recombinantly fused with a Cas12J polypeptide include MQ1 and Sss1. Cas12J polypeptides may also be adapted for use in a SunTag system for a particular application (WO2016011070). In some embodiments, a dCas12J polypeptide may include a tag to allow for visualization of various subcellular locations (e.g. DNA sequence, such as e.g. 180 bp repeats for chromocenters).

Linkers

Various linkers may be used in the construction of recombinant proteins as described herein. In general, linkers are short peptides that separate the different domains in a multi-domain protein. They may play an important role in fusion proteins, affecting the crosstalk between the different domains, the yield of protein production, and the stability and/or the activity of the fusion proteins. Linkers are generally classified into 2 major categories: flexible or rigid. Flexible linkers are typically used when the fused domains require a certain degree of movement or interaction, and these linkers are usually composed of small amino acids such as, for example, glycine (G), serine (S) or proline (P).

The certain degree of movement between domains allowed by flexible linkers is an advantage in some fusion proteins. However, it has been reported that flexible linkers can sometimes reduce protein activity due to an inefficient separation of the two domains. In this case, rigid linkers may be used since they enforce a fixed distance between domains and promote their independent functions. A thorough description of several linkers has been provided in Chen X et al., 2013, Advanced Drug Delivery Reviews 65 (2013) 1357-1369).

Various linkers may be used in, for example, the construction of recombinant polypeptides as described herein. Linkers may be used in e.g. Cas12J fusion proteins as described herein to separate the coding sequences of the Cas12J polypeptide and the other polypeptide recombinantly fused to Cas12J. For example, a variety of wiggly/flexible linkers, stiff/rigid linkers, short linkers, and long linkers may be used as described herein. Various linkers as described herein may be used in the construction of recombinant proteins as described herein.

A variety of shorter or longer linker regions are known in the art, for example corresponding to a series of glycine residues, a series of adjacent glycine-serine dipeptides, a series of adjacent glycine-glycine-serine tripeptides, or known linkers from other proteins. A flexible linker may include, for example, the amino acid sequence: SSGPPPGTG (SEQ ID NO: 88) and variants thereof. A rigid linker may include, for example, the amino acid sequence: AEAAAKEAAAKA (SEQ ID NO: 89) and variants thereof. The XTEN linker, SGSETPGTSESATPES (SEQ ID NO: 90), and variants thereof, described in Guilinget et al, 2014 (Nature Biotechnology 32, 577-582), may also be used.

Nuclear Localization Signals (NLS)

Recombinant polypeptides of the present disclosure may contain one or more nuclear localization signals (NLS). Nuclear localization signals may also be referred to as nuclear localization sequences, domains, peptides, or other terms readily apparent to those of skill in the art. Nuclear localization signals are a translocation sequence that, when present in a polypeptide, direct that polypeptide to localize to the nucleus of a eukaryotic cell.

Various nuclear localization signals may be used in recombinant polypeptides of the present disclosure. For example, one or more SV40-type NLS or one or more REX NLS may be used in recombinant polypeptides. Recombinant polypeptides may also contain two or more tandem copies of a nuclear localization signal. For example, recombinant polypeptides may contain at least two, at least three, at least for, at least five, at least six, at least seven, at least eight, at least nine, or at least ten copies, either tandem or not, of a nuclear localization signal.

Recombinant polypeptides of the present disclosure may contain one or more nuclear localization signals that contain an amino acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 19 and/or SEQ ID NO: 20.

Tags, Reporters, and Other Features

Recombinant polypeptides of the present disclosure may contain one or more tags that allow for e.g. purification and/or detection of the recombinant polypeptide. Various tags may be used herein and are well-known to those of skill in the art. Exemplary tags may include HA, GST, FLAG, MBP, etc., and multiple copies of one or more tags may be present in a recombinant polypeptide.

Recombinant polypeptides of the present disclosure may contain one or more reporters that allow for e.g. visualization and/or detection of the recombinant polypeptide. A reporter polypeptide encodes a protein that may be readily detectable due to its biochemical characteristics such as, for example, enzymatic activity or chemifluorescent features. Reporter polypeptides may be detected in a number of ways depending on the characteristics of the particular reporter. For example, a reporter polypeptide may be detected by its ability to generate a detectable signal (e.g. fluorescence), by its ability to form a detectable product, etc. Various reporters may be used herein and are well-known to those of skill in the art. Exemplary reporters may include GFP, GUS, mCherry, luciferase, etc., and multiple copies of one or more tags may be present in a recombinant polypeptide.

Recombinant polypeptides of the present disclosure may contain one or more polypeptide domains that serve a particular purpose depending on the particular goal/need. For example, recombinant polypeptides may contain a GB1 polypeptide. Recombinant polypeptides may contain translocation sequences that target the polypeptide to a particular cellular compartment or area. Suitable features will be readily apparent to those of skill in the art.

### Recombinant Nucleic Acids

Certain aspects of the present disclosure relate to recombinant nucleic acids. In some embodiments, recombinant nucleic acids encode recombinant polypeptides of the present disclosure.

As used herein, the terms “polynucleotide,” “nucleic acid,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications. As used herein, the symbols for nucleotides and polynucleotides are those recommended by the IUPAC-IUB Commission of Biochemical Nomenclature.

“Recombinant nucleic acid” or “heterologous nucleic acid” or “recombinant polynucleotide” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids contains two or more subsequences that are not found in the same relationship to each other in nature. For example, regarding instance (c), a recombinant nucleic acid sequence will have two or more sequences from unrelated genes arranged to make a new functional nucleic acid. In some embodiments, the present disclosure describes the introduction of an expression vector into a plant cell, where the expression vector contains a nucleic acid sequence coding for a protein that is not normally found in a plant cell or contains a nucleic acid coding for a protein that is normally found in a plant cell but is under the control of different regulatory sequences. With reference to the plant cell's genome, then, the nucleic acid sequence that codes for the protein is recombinant. A protein that is referred to as recombinant may be encoded by a recombinant nucleic acid sequence which may be present in the plant cell. Recombinant proteins of the present disclosure may also be exogenously supplied directly to host cells (e.g. plant cells).

In some embodiments, a recombinant nucleic acid is provided that encodes a recombinant Cas12J polypeptide. In some embodiments, the recombinant nucleic acid encodes a Cas12J polypeptide that has an amino acid sequence that is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 2.

In some embodiments, a recombinant nucleic acid may encode a vector or a portion of a vector that contains a nucleic acid sequence encoding a Cas12J polypeptide. For example, recombinant nucleic acids are provided that have a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the nucleic acid sequence of any one of SEQ ID NO: 13 or SEQ ID NO: 14.

Sequences of the polynucleotides of the present disclosure may be prepared by various suitable methods known in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteucci et al., (1980) Tetrahedron Lett 21:719-722; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired polynucleotide sequence from the gel via techniques known to those of ordinary skill in the art, such as utilization of polymerase chain reactions (PCR; e.g., U.S. Pat. No. 4,683,195).

The nucleic acids employed in the methods and compositions described herein may be codon optimized relative to a parental template for expression in a particular host cell. Cells differ in their usage of particular codons, and codon bias corresponds to relative abundance of particular tRNAs in a given cell type. By altering codons in a sequence so that they are tailored to match with the relative abundance of corresponding tRNAs, it is possible to increase expression of a product (e.g. a polypeptide) from a nucleic acid. Similarly, it is possible to decrease expression by deliberately choosing codons corresponding to rare tRNAs. Thus, codon optimization/deoptimization can provide control over nucleic acid expression in a particular cell type (e.g. bacterial cell, plant cell, mammalian cell, etc.). Methods of codon optimizing a nucleic acid for tailored expression in a particular cell type are well-known to those of skill in the art.

Guide RNAs

Certain aspects of the present disclosure relate to guide RNAs and their use in CRISPR-based targeting of a target nucleic acid. Guide RNAs of the present disclosure are capable of binding or otherwise interacting with a Cas12J polypeptide to facilitate targeting of the Cas12J polypeptide to a target nucleic acid. Suitable and exemplary guide RNAs are provided herein and design of such to target a particular nucleic acid will be readily apparent to one of skill in the art. Guide RNAs may also be modified to improve the efficiency of their function in guiding Cas12J to a target nucleic acid.

Guide RNAs of the present disclosure contain a CRISPR RNA (crRNA) sequence, and the sequence of the crRNA is involved in conferring specificity to targeting a specific nucleic acid sequence.

In some embodiments, guide RNA molecules may be extended to include sites for the binding of RNA binding proteins. In some embodiments, multiple guide RNAs can be assembled into a pre-crRNA array that can be processed by the RuvC domain of Cas12J. This will allow for multiplex editing to enable simultaneous targeting to several sites.

In some embodiments, a guide RNA contains both RNA and a repeat sequence that is composed of DNA. In this sense, a guide RNA may be an RNA-DNA hybrid molecule.

A guide RNA (gRNA) may be expressed in a variety of ways as will be apparent to one of skill in the art. For example, a gRNA may be expressed from a recombinant nucleic acid in vivo, from a recombinant nucleic acid in vitro, from a recombinant nucleic acid ex vivo, or can be synthetically synthesized.

A guide RNA of the present disclosure may have various nucleotide lengths. A guide RNA may contain, for example, at least 5, at least 10, at least 15, at least 20, at least 25, at least 30, at least 35, at least 40, at least 50, at least 60, at least 70, at least 80, at least 90, at least 100, at least 110, at least 120, at least 130, at least 140, at least 150, at least 160, at least 170, at least 180 nucleotides, at least 190 nucleotides, or at least 200 nucleotides or more. Longer guide RNAs may result in increased editing efficiency by Cas12J polypeptides.

A guide RNA of the present disclosure may hybridize with a particular nucleotide sequence on a target nucleic acid. This hybridization may be 100% complimentary or it may be less than 100% complimentary so long as the hybridization is sufficient to allow Cas12J to bind to or interact with the target nucleic acid. A guide RNA may contain a nucleotide sequence that is, for example, at least 80%, at least 85%, at least 90%, at least 91%, at least 92%, at least 93%, at least 94%, at least 95%, at least 96%, at least 97%, at least 98%, at least 99%, or 100% identical or complimentary to the target nucleotide sequence in the target nucleic acid that is targeted by/to be hybridized with the guide RNA.

In some embodiments, increasing expression of a guide RNA may increase the editing efficiency of a target nucleic acid according to the methods of the present disclosure. In some embodiments, use of a Pol II promoter (e.g. a CmYLCV promoter) to drive gRNA expression may result in increased expression of the guide RNA as compared to a corresponding control promoter (e.g. a Pol III promoter, such as a U6 promoter for example). Use of a Pol II promoter to drive gRNA expression may increase the expression of the guide RNA by, for example, at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more as compared to a corresponding control (e.g. a U6 promoter).

In some embodiments, a guide RNA of the present disclosure may be recombinantly fused with a ribozyme sequence to assist in gRNA processing. Exemplary ribozymes for use herein will be readily apparent to one of skill in the art. Exemplary ribozymes may include, for example, a Hammerhead-type ribozyme and a hepatitis delta virus ribozyme. Use of a ribozyme to assist in processing of guide RNAs may increase efficiency of editing of a target nucleic acid sequence by a Cas12J polypeptide of the present disclosure. Use of a ribozyme fused to a gRNA may increase relative editing efficiency by, for example, at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more as compared to a corresponding control (e.g. a guide RNA that is expressed without the assistance of any additional processing machinery).

### Methods of Identifying Sequence Similarity

Various methods are known to those of skill in the art for identifying similar (e.g. homologs, orthologs, paralogs, etc.) polypeptide and/or polynucleotide sequences, including phylogenetic methods, sequence similarity analysis, and hybridization methods.

Phylogenetic trees may be created for a gene family by using a program such as CLUSTAL (Thompson et al. Nucleic Acids Res. 22: 4673-4680 (1994); Higgins et al. Methods Enzymol 266: 383-402 (1996)) or MEGA (Tamura et al. Mol. Biol. & Evo. 24:1596-1599 (2007)). Once an initial tree for genes from one species is created, potential orthologous sequences can be placed in the phylogenetic tree and their relationships to genes from the species of interest can be determined. Evolutionary relationships may also be inferred using the Neighbor-Joining method (Saitou and Nei, Mol. Biol. & Evo. 4:406-425 (1987)). Homologous sequences may also be identified by a reciprocal BLAST strategy. Evolutionary distances may be computed using the Poisson correction method (Zuckerkandl and Pauling, pp. 97-166 in Evolving Genes and Proteins, edited by V. Bryson and H. J. Vogel. Academic Press, New York (1965)).

In addition, evolutionary information may be used to predict gene function. Functional predictions of genes can be greatly improved by focusing on how genes became similar in sequence (i.e. by evolutionary processes) rather than on the sequence similarity itself (Eisen, Genome Res. 8: 163-167 (1998)). Many specific examples exist in which gene function has been shown to correlate well with gene phylogeny (Eisen, Genome Res. 8: 163-167 (1998)). By using a phylogenetic analysis, one skilled in the art would recognize that the ability to deduce similar functions conferred by closely-related polypeptides is predictable.

When a group of related sequences are analyzed using a phylogenetic program such as CLUSTAL, closely related sequences typically cluster together or in the same clade (a group of similar genes). Groups of similar genes can also be identified with pair-wise BLAST analysis (Feng and Doolittle, J. Mol. Evol. 25: 351-360 (1987)). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example. Mount. Bioinformatics: Sequence and Genome Analysis Cold Spring Harbor Laboratory Press, Cold Spring Harbor, N.Y., page 543 (2001)).

To find sequences that are homologous to a reference sequence. BLAST nucleotide searches can be performed with the BLASTN program, score=100, wordlength=12, to obtain nucleotide sequences homologous to a nucleotide sequence encoding a protein of the disclosure. BLAST protein searches can be performed with the BLASTX program, score=50, wordlength=3, to obtain amino acid sequences homologous to a protein or polypeptide of the disclosure. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST. Gapped BLAST, or PSI-BLAST, the default parameters of the respective programs (e.g., BLASTN for nucleotide sequences, BLASTX for proteins) can be used.

Methods for the alignment of sequences and for the analysis of similarity and identity of polypeptide and polynucleotide sequences are well-known in the art.

As used herein “sequence identity” refers to the percentage of residues that are identical in the same positions in the sequences being analyzed. As used herein “sequence similarity” refers to the percentage of residues that have similar biophysical/biochemical characteristics in the same positions (e.g. charge, size, hydrophobicity) in the sequences being analyzed.

Methods of alignment of sequences for comparison are well-known in the art, including manual alignment and computer assisted sequence alignment and analysis. This latter approach is a preferred approach in the present disclosure, due to the increased throughput afforded by computer assisted methods. As noted below, a variety of computer programs for performing sequence alignment are available, or can be produced by one of skill.

The determination of percent sequence identity and/or similarity between any two sequences can be accomplished using a mathematical algorithm. Examples of such mathematical algorithms are the algorithm of Myers and Miller, CABIOS 4:11-17 (1988); the local homology algorithm of Smith et al., Adv. Appl. Math. 2:482 (1981); the homology alignment algorithm of Needleman and Wunsch, J. Mol. Biol. 48:443-453 (1970); the search-for-similarity-method of Pearson and Lipman, Proc. Natl. Acad. Sci. 85:2444-2448 (1988); the algorithm of Karlin and Altschul, Proc. Natl. Acad. Sci. USA 87:2264-2268 (1990), modified as in Karlin and Altschul. Proc. Natl. Acad. Sci. USA 90:5873-5877 (1993).

Computer implementations of these mathematical algorithms can be utilized for comparison of sequences to determine sequence identity and/or similarity. Such implementations include, for example: CLUSTAL in the PC/Gene program (available from Intelligenetics, Mountain View, Calif.); the AlignX program, version10.3.0 (Invitrogen, Carlsbad, Calif.) and GAP, BESTFIT, BLAST, FASTA, and TFASTA in the Wisconsin Genetics Software Package, Version 8 (available from Genetics Computer Group (GCG), 575 Science Drive, Madison. Wis., USA). Alignments using these programs can be performed using the default parameters. The CLUSTAL program is well described by Higgins et al. Gene 73:237-244 (1988); Higgins et al. CABIOS 5:151-153 (1989); Corpet et al., Nucleic Acids Res. 16:10881-90 (1988); Huang et al. CABIOS 8:155-65 (1992); and Pearson et al., Meth. Mol. Biol. 24:307-331 (1994). The BLAST programs of Altschul et al. J. Mol. Biol. 215:403-410 (1990) are based on the algorithm of Karlin and Altschul (1990) supra.

Polynucleotides homologous to a reference sequence can be identified by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in references cited below (e.g., Sambrook et al., Molecular Cloning: A Laboratory Manual, 2nd Ed., Vol. 1-3, Cold Spring Harbor Laboratory, Cold Spring Harbor. N.Y. (“Sambrook”) (1989); Berger and Kimmel, Guide to Molecular Cloning Techniques, Methods in Enzymology, vol. 152 Academic Press, Inc., San Diego, Calif. (“Berger and Kimmel”) (1987); and Anderson and Young, “Quantitative Filter Hybridisation.” In: Hames and Higgins, ed., Nucleic Acid Hybridisation, A Practical Approach. Oxford, TRL Press, 73-111 (1985)).

Encompassed by the disclosure are polynucleotide sequences that are capable of hybridizing to the disclosed polynucleotide sequences and fragments thereof under various conditions of stringency (see, for example, Wahl and Berger, Methods Enzymol. 152: 399-407 (1987); and Kimmel, Methods Enzymo. 152: 507-511, (1987)). Full length cDNA, homologs, orthologs, and paralogs of polynucleotides of the present disclosure may be identified and isolated using well-known polynucleotide hybridization methods.

With regard to hybridization, conditions that are highly stringent, and means for achieving them, are well known in the art. See, for example. Sambrook et al. (1989) (supra); Berger and Kimmel (1987) pp. 467-469 (supra); and Anderson and Young (1985)(supra).

Hybridization experiments are generally conducted in a buffer of pH between 6.8 to 7.4, although the rate of hybridization is nearly independent of pH at ionic strengths likely to be used in the hybridization buffer (Anderson and Young (1985)(supra)). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency. As a general guideline, high stringency is typically performed at Tm-5° C. to Tm-20° C., moderate stringency at Tm-20° C. to Tm-35° C. and low stringency at Tm-35° C. to Tm-50° C. for duplex>150 base pairs. Hybridization may be performed at low to moderate stringency (25-50° C. below Tm), followed by post-hybridization washes at increasing stringencies. Maximum rates of hybridization in solution are determined empirically to occur at Tm-25° C. for DNA-DNA duplex and Tm-15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tm) for the specific sequence at a defined ionic strength and pH.

Hybridization and wash conditions that may be used to bind and remove polynucleotides with less than the desired homology to the nucleic acid sequences or their complements of the present disclosure include, for example: 6×SSC and 1% SDS at 65° C.; 50% formamide, 4×SSC at 42° C.; 0.5×SSC to 2.0×SSC, 0.1% SDS at 50° C. to 65° C.; or 0.1×SSC to 2×SSC, 0.1% SDS at 50° C.-65° C.; with a first wash step of, for example, 10 minutes at about 42° C. with about 20% (v/v) formamide in 0.1×SSC, and with, for example, a subsequent wash step with 0.2×SSC and 0.1% SDS at 65° C. for 10, 20 or 30 minutes.

For identification of less closely related homologs, wash steps may be performed at a lower temperature, e.g., 50° C. An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min. Wash procedures will generally employ at least two final wash steps. Additional variations on these conditions will be readily apparent to those skilled in the art (see, for example, US Patent Application No. 20010010913).

If desired, one may employ wash steps of even greater stringency, including conditions of 65° C.-68° C. in a solution of 15 mM NaCl, 1.5 mM trisodium citrate, and 0.1% SDS, or about 0.2×SSC, 0.1% SDS at 65° C. and washing twice, each wash step of 10, 20 or 30 min in duration, or about 0.1×SSC, 0.1% SDS at 65° C. and washing twice for 10, 20 or 30 min. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C.

### Target Nucleic Acids and Sequences

Cas12J polypeptides of the present disclosure may be targeted to specific target nucleic acids to modify the target nucleic acid. As described above, Cas12J is targeted to a target nucleic acid based on its association/complex with a guide RNA that is able to hybridize with the particular target nucleotide sequence in the target nucleic acid. In this sense, the guide RNA provides the targeting functionality to target a particular target nucleotide sequence in a target nucleic acid. Various types of nucleic acids may be targeted to e.g. modulate their expression, as will be readily apparent to one of skill in the art.

Certain aspects of the present disclosure relate to targeting a target nucleic acid with a Cas12J polypeptide such that the Cas12J polypeptide is able to enact enzymatic activity at the target nucleic acid. In some embodiments, a Cas12J polypeptide/gRNA complex is targeted to a target nucleic acid and introduces an edit/modification into the target nucleic acid. In some embodiments, the edit/modification is to introduce a single-stranded break or a double stranded break into the nucleic acid backbone of the target nucleic acid.

Certain aspects of the present disclosure relate to target sites on target nucleic acids. A target site generally refers to a location of a target nucleic acid that is capable of being bound by a Cas12J/gRNA complex and subjected to the activity of a Cas12J polypeptide or variant thereof. In some embodiments, the target site may include both the nucleotide sequence hybridized with a guide RNA as well as at least 5, at least 10, at least 15, at least 20, at least 25, at least 30, at least 35, at least 40, at least 45, or at least 50 nucleotides or more on the 3′ side, the 5′ side, or both the 3′ and 5′ side of the nucleotide sequence in the target nucleic acid that is hybridized with a guide RNA. In some embodiments, the target site may contain at least 30, at least 40, at least 50, at least 60, at least 70, at least 80, at least 90, at least 100, at least 125, at least 150, at least 175, or at least 200 or more nucleotides.

In some embodiments, a Cas12J polypeptide is targeted to a particular locus. A locus generally refers to a specific position on a chromosome or other nucleic acid molecule. A locus may contain, for example, a polynucleotide that encodes a protein or an RNA. A locus may also contain, for example, a non-coding RNA, a gene, a promoter, a 5′ untranslated region (UTR), an exon, an intron, a 3′ UTR, or combinations thereof. In some embodiments, a locus may contain a coding region for a gene.

In some embodiments, a Cas12J polypeptide is targeted to a gene. A gene generally refers to a polynucleotide that can produce a functional unit (for example, a protein or a noncoding RNA molecule). A gene may contain a promoter, an enhancer sequence, a leader sequence, a transcriptional start site, a transcriptional stop site, a polyadenylation site, one or more exons, one or more introns, a 5′ UTR, a 3′ UTR, or combinations thereof. A gene sequence may contain a polynucleotide sequence encoding a promoter, an enhancer sequence, a leader sequence, a transcriptional start site, a transcriptional stop site, a polyadenylation site, one or more exons, one or more introns, a 5′ UTR, a 3′ UTR, or combinations thereof.

The target nucleic acid sequence may be located within the coding region of a target gene or upstream or downstream thereof. Moreover, the target nucleic acid sequence may reside endogenously in a target gene or may be inserted into the gene, e.g., heterologous, for example, using techniques such as homologous recombination. For example, a target gene of the present disclosure can be operably linked to a control region, such as a promoter, that contains a sequence that can be recognized by a guide RNA of the present disclosure such that a Cas12J polypeptide may be targeted to that sequence.

The target nucleic acid sequence may be located in a region of chromatin. In some embodiments, the target nucleic acid sequence to be edited by a Cas12J polypeptide may be in a region of open chromatin or similar region of DNA that is generally accessible to transcriptional machinery. Regions of open chromatin may be characterized by nucleosome depletion, nucleosome disruption, accessibility to transcriptional machinery, and/or a transcriptionally active state. Regions of open chromatin will be readily understood and identifiable by one of skill in the art. Editing a target nucleic acid sequence that is in a region of open chromatin may result in improved editing efficiency by the Cas12J polypeptide as compared to a corresponding control nucleic acid sequence (e.g. one that is present in a region of more closed, repressive, and/or transcriptionally inactive chromatin).

Target genes or nucleic acid regions to be edited by a Cas12J polypeptide of the present disclosure will be readily apparent to those of skill in the art depending on the particular application and/or purpose. For example, genes with particular agricultural importance may be edited/modified according to the methods of the present disclosure. Exemplary genes to be edited/modified may include, for example, those involved in light perception (e.g. PHYB, etc.), those involved in the circadian clock (e.g. CCA1, LHY, etc.), those involved in flowering time (e.g. CO, FT, etc.), those involved in meristem size (e.g. WUS, CLV3, etc.), those involved in plant architecture (S, SP, TFLI, SFT, etc.) and genes involved in embryogenesis, chromatin structure, stress response, growth and development, etc.

In some embodiments, the target nucleic acid is endogenous to the plant where the expression of one or more genes is modulated according to the methods described herein. In some embodiments, the target nucleic acid is a transgene of interest that has been inserted into a plant. Suitable target nucleic acids will be readily apparent to one of skill in the art depending on the particular need or outcome. The target nucleic acid sequence may be in e.g. a region of euchromatin (e.g. highly expressed gene), or the target nucleic acid sequence may be in a region of heterochromatin (e.g. centromere DNA).

In some embodiments, the target nucleic acid may be in a region of repressive chromatin. Repressive chromatin generally refers to regions of chromatin where transcription is repressed or otherwise generally transcriptionally inactive. Exemplary regions of repressive chromatin include, for example, regions with repressive DNA methylation, compact chromatin, and/or no transcription).

In some embodiments, recombinant Cas12J polypeptides of the present disclosure can be used to create mutations in plants that result in reduced or silenced expression of a target gene. In some embodiments, recombinant Cas12J polypeptides of the present disclosure can be used to create functional “overexpression” mutations in a plant by releasing repression of the target gene expression as a consequence of a modification that results in transcriptional activation of the target nucleic acid. Release of gene expression repression, which may lead to activation of gene expression, may be of a structural gene, e.g., one encoding a protein having for example enzymatic activity, or of a regulatory gene, e.g., one encoding a protein that in turn regulates expression of a structural gene.

### Recombinant Expression

Recombinant nucleic acids and/or recombinant polypeptides of the present disclosure may be present in host cells (e.g. plant cells). In some embodiments, recombinant nucleic acids are present in an expression vector and may encode a recombinant polypeptide, and the expression vector may be present in host cells (e.g. plant cells). In some embodiments, recombinant nucleic acids and/or recombinant polypeptides are present in host cells (e.g. plant cells) via direct introduction into the cell (e.g. via RNPs).

In some embodiments, the genes encoding the recombinant polypeptides in the plant cell may be heterologous to the plant cell. In certain embodiments, the plant cell does not naturally produce one or more polypeptides of the present disclosure, and contains heterologous nucleic acid constructs capable of expressing one or more genes necessary for producing those molecules. In certain embodiments, the plant cell does not naturally produce one or more polypeptides of the present disclosure, and is provided the one or more polypeptides through exogenous delivery of the polypeptides directly to the plant cell without the need to express a recombinant nucleic acid encoding the recombinant polypeptide in the plant cell.

Recombinant polypeptides of the present disclosure may be introduced into host cells (e.g. plant cells) via any suitable methods known in the art. For example, a recombinant Cas12J polypeptide can be exogenously added to plant cells and the plant cells are maintained under conditions such that the recombinant polypeptide is targeted (via a guide RNA) to one or more target nucleic acids to edit/modify the target nucleic acids in the plant cells. Alternatively, a recombinant nucleic acid encoding a recombinant Cas12J polypeptide of the present disclosure can be expressed in plant cells and the plant cells are maintained under conditions such that the recombinant Cas12J polypeptide is targeted (via a guide RNA) to one or more target nucleic acids to edit/modify the target nucleic acids in the plant cells. Additionally, in some embodiments, a recombinant Cas12J polypeptide of the present disclosure may be transiently expressed in a plant via viral infection of the plant, or by introducing a recombinant Cas12J polypeptide-encoding RNA into a plant to facilitate editing/modification of a target nucleic acid of interest. This approach may be particularly well-suited for Cas12J-based editing given that the small size of Cas12J proteins may make them more amenable to delivery via virus. Methods of introducing recombinant proteins via viral infection or via the introduction of RNAs into plants are well known in the art. For example, Tobacco rattle virus (TRV) has been successfully used to introduce zinc finger nucleases in plants to cause genome modification (“Nontransgenic Genome Modification in Plant Cells”, Plant Physiology 154:1079-1087 (2010)). TRV and other appropriate viruses may be used herein to facilitate editing in plants cells.

In some embodiments, a Cas12J polypeptide and a guide RNA may be exogenously and directly supplied to a plant cell as a ribonucleoprotein (RNP) complex. This particular form of delivery is useful for facilitating transgene-free editing in plants. Modified guide RNAs which are resistant to nuclease digestion could also be used in this approach. Transgene-free callus from plants cells provided with an RNP could be used to regenerate whole edited plants.

A recombinant nucleic acid encoding a recombinant polypeptide of the present disclosure can be expressed in a plant with any suitable plant expression vector. Typical vectors useful for expression of recombinant nucleic acids in higher plants are well known in the art and include, for example, vectors derived from the tumor-inducing (Ti) plasmid of Agrobacterium tumefaciens (e.g., see Rogers et al., Meth. in Enzymol. (1987) 153:253-277). These vectors are plant integrating vectors in that on transformation, the vectors integrate a portion of vector DNA into the genome of the host plant. Exemplary A. tumefaciens vectors useful herein are plasmids pKYLX6 and pKYLX7 (e.g., see of Schardl et al., Gene (1987) 61:1-11; and Berger et al., Proc. Natl. Acad. Sci. USA (1989) 86:8402-8406); and plasmid pBI 101.2 that is available from Clontech Laboratories, Inc. (Palo Alto, Calif.).

In addition to regulatory domains, recombinant polypeptides of the present disclosure can be expressed as a fusion protein that is coupled to, for example, a maltose binding protein (“MBP”), glutathione S transferase (GST), hexahistidine, c-myc, or the FLAG epitope for ease of purification, monitoring expression, or monitoring cellular and subcellular localization.

Moreover, a recombinant nucleic acid encoding a recombinant polypeptide of the present disclosure can be modified to improve expression of the recombinant protein in plants by using codon preference/codon optimization to target preferential expression in plant cells. When the recombinant nucleic acid is prepared or altered synthetically, advantage can be taken of known codon preferences of the intended plant host where the nucleic acid is to be expressed. For example, recombinant nucleic acids of the present disclosure can be modified to account for the specific codon preferences and GC content preferences of monocotyledons and dicotyledons, as these preferences have been shown to differ (Murray et al., Nucl. Acids Res. (1989) 17: 477-498).

The present disclosure further provides expression vectors encoding recombinant polypeptides of the present disclosure. A nucleic acid sequence coding for the desired recombinant nucleic acid of the present disclosure can be used to construct a recombinant expression vector which can be introduced into the desired host cell. A recombinant expression vector will typically contain a nucleic acid encoding a recombinant protein of the present disclosure, operably linked to transcriptional initiation regulatory sequences which will direct the transcription of the nucleic acid in the intended host cell, such as tissues of a transformed plant.

Recombinant nucleic acids e.g. encoding recombinant polypeptides of the present disclosure may be expressed on multiple expression vectors or they may be expressed on a single expression vector. For example, plant expression vectors may include (1) a cloned gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

In some embodiments, expression of a nucleic acid of the present disclosure may be driven (in operable linkage) with a promoter (e.g. a promoter functional in plants or a plant-specific promoter). A promoter generally refers to a DNA sequence that contains an RNA polymerase binding site, transcription start site, and/or TATA box and assists or promotes the transcription and expression of an associated transcribable polynucleotide sequence such as, for example, a gene. A plant promoter, or functional fragment thereof, can be employed to e.g. control the expression of a recombinant nucleic acid of the present disclosure in regenerated plants. The selection of the promoter used in expression vectors will determine the spatial and temporal expression pattern of the recombinant nucleic acid in the modified plant, e.g., the nucleic acid encoding the recombinant polypeptide of the present disclosure is only expressed in the desired tissue or at a certain time in plant development or growth. Certain promoters will express recombinant nucleic acids in all plant tissues and are active under most environmental conditions and states of development or cell differentiation (i.e., constitutive promoters). Other promoters will express recombinant nucleic acids in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the recombinant nucleic acid under various inducing conditions.

Examples of suitable constitutive promoters may include, for example, the core promoter of the Rsyn7, the core CaMV 35S promoter (Odell et al., Nature (1985) 313:810-812), CaMV 19S (Lawton et al., 1987), rice actin (Wang et al., 1992; U.S. Pat. No. 5,641,876; and McElroy et al., Plant Cell (1985) 2:163-171); ubiquitin (Christensen et al., Plant Mol. Biol. (1989)12:619-632; and Christensen et al., Plant Mol. Biol. (1992) 18:675-689), pEMU (Last et al., Theor. Appl. Genet. (1991) 81:581-588), MAS (Velten et al., EMBO J. (1984) 3:2723-2730), nos (Ebert et al., 1987), Adh (Walker et al., 1987), the P- or 2′-promoter derived from T-DNA of Agrobacterium tumefaciens, the Smas promoter, the cinnamyl alcohol dehydrogenase promoter (U.S. Pat. No. 5,683,439), the Nos promoter, the pEmu promoter, the rubisco promoter, the GRP 1-8 promoter, and other transcription initiation regions from various plant genes known to those of skilled artisans, and constitutive promoters described in, for example, U.S. Pat. Nos. 5,608,149; 5,608,144; 5,604,121; 5,569,597; 5,466,785; 5,399,680; 5,268,463; and 5,608,142.

In some embodiments, expression of a nucleic acid of the present disclosure may be driven (in operable linkage) with a UBQ10 promoter. In some embodiments, expression of a nucleic acid of the present disclosure may be driven (in operable linkage) with a promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the nucleic acid sequence of SEQ ID NO: 23.

Recombinant nucleic acids of the present disclosure may be expressed using an RNA Polymerase III (Pol III) promoter such as, for example, the U6 promoter or the H1 promoter (eLife 2013 2:e00471). For example, an approach in plants has been described using three different Pol III promoters from three different Arabidopsis U6 genes, and their corresponding gene terminators (BMC Plant Biology 2014 14:327). One skilled in the art would readily understand that many additional Pol III promoters could be utilized to, for example, simultaneously express many guide RNAs to many different locations in the genome simultaneously. The use of different Pol III promoters for each gRNA expression cassette may be desirable to reduce the chances of natural gene silencing that can occur when multiple copies of identical sequences are expressed in plants.

In some embodiments, expression of a nucleic acid of the present disclosure may be driven (in operable linkage) with a U6 promoter. In some embodiments, expression of a nucleic acid of the present disclosure may be driven (in operable linkage) with a promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the nucleic acid sequence of SEQ ID NO: 24.

Recombinant nucleic acids of the present disclosure may be expressed using an RNA Polymerase II (Pol II) promoter such as, for example, the CmYLCV promoter and the 35S promoter. Use of a Pol II promoter to drive expression of nucleic acids (e.g. guide RNA expression) may provide additional flexibility for controlling the strength/degree of expression and may provide the possibility of tissue-specific expression. One skilled in the art would recognize appropriate Pot II promoters for use in the methods and compositions of the present disclosure.

In some embodiments, expression of a nucleic acid of the present disclosure may be driven (in operable linkage) with a CmYLCV promoter. In some embodiments, expression of a nucleic acid of the present disclosure may be driven (in operable linkage) with a promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the nucleic acid sequence of SEQ ID NO: 29.

In some embodiments, expression of a nucleic acid of the present disclosure may be driven (in operable linkage) with a 2×35S promoter. In some embodiments, expression of a nucleic acid of the present disclosure may be driven (in operable linkage) with a promoter having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the nucleic acid sequence of SEQ ID NO: 34.

Examples of suitable tissue specific promoters may include, for example, the lectin promoter (Vodkin et al., 1983; Lindstrom et al., 1990), the corn alcohol dehydrogenase 1 promoter (Vogel et al., 1989; Dennis et al., 1984), the corn light harvesting complex promoter (Simpson, 1986; Bansal et al., 1992), the corn heat shock protein promoter (Odell et al., Nature (1985) 313:810-812; Rochester et al., 1986), the pea small subunit RuBP carboxylase promoter (Poulsen et al., 1986; Cashmore et al., 1983), the Ti plasmid mannopine synthase promoter (Langridge et al., 1989), the Ti plasmid nopaline synthase promoter (Langridge et al., 1989), the petunia chalcone isomerase promoter (Van Tunen et al., 1988), the bean glycine rich protein 1 promoter (Keller et al., 1989), the truncated CaMV 35s promoter (Odell et al., Nature (1985) 313:810-812), the potato patatin promoter (Wenzler et al., 1989), the root cell promoter (Conkling et al., 1990), the maize zein promoter (Reina et al., 1990; Kriz et al., 1987; Wandelt and Feix, 1989; Langridge and Feix, 1983; Reina et al., 1990), the globulin-1 promoter (Belanger and Kriz et al., 1991), the α-tubulin promoter, the cab promoter (Sullivan et al., 1989), the PEPCase promoter (Hudspeth & Grula, 1989), the R gene complex-associated promoters (Chandler et al., 1989), and the chalcone synthase promoters (Franken et al., 1991).

Alternatively, the plant promoter can direct expression of a recombinant nucleic acid of the present disclosure in a specific tissue or may be otherwise under more precise environmental or developmental control. Such promoters are referred to here as “inducible” promoters. Environmental conditions that may affect transcription by inducible promoters include, for example, pathogen attack, anaerobic conditions, or the presence of light. Examples of inducible promoters include, for example, the AdhI promoter which is inducible by hypoxia or cold stress, the Hsp70 promoter which is inducible by heat stress, and the PPDK promoter which is inducible by light. Examples of promoters under developmental control include, for example, promoters that initiate transcription only, or preferentially, in certain tissues, such as leaves, roots, fruit, seeds, or flowers. An exemplary promoter is the anther specific promoter 5126 (U.S. Pat. Nos. 5,689,049 and 5,689,051). The operation of a promoter may also vary depending on its location in the genome. Thus, an inducible promoter may become fully or partially constitutive in certain locations.

Moreover, any combination of a constitutive or inducible promoter, and a non-tissue specific or tissue specific promoter may be used to control the expression of various recombinant polypeptides of the present disclosure.

The recombinant nucleic acids of the present disclosure and/or a vector housing a recombinant nucleic acid of the present disclosure, may also contain a regulatory sequence that serves as a 3′ terminator sequence. A terminator sequence generally refers to a nucleic acid sequence that marks the end of a gene or transcribable nucleic acid during transcription. One of skill in the art would readily recognize a variety of terminators that may be used in the recombinant nucleic acids of the present disclosure. For example, a recombinant nucleic acid of the present disclosure may contain a 3′ NOS terminator. In some embodiments, recombinant nucleic acids of the present disclosure contain a transcriptional termination site. Transcription termination sites may include, for example, OCS terminators, rbcS-E9 terminators, NOS terminators, HSP18.2 terminators, and poly-T terminators.

In some embodiments, a nucleic acid of the present disclosure may contain a transcriptional termination site having a nucleic acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the nucleic acid sequence of SEQ ID NO: 30 (a 35S terminator), SEQ ID NO: 35 (a HSP18 terminator), and/or SEQ ID NO: 40 (an RbcS-E9 terminator).

Recombinant nucleic acids of the present disclosure may include one or more introns. Introns may be included in e.g. recombinant nucleic acids being expressed on a vector in a host cell. The inclusion of one of more introns in a recombinant nucleic acid to be expressed may be particularly helpful to increase expression in plant cells.

Recombinant nucleic acids of the present disclosure may also contain selectable markers. A selectable marker can be used to assist in the selection of transformed cells or tissue due to the presence of a selection agent, such as an antibiotic or herbicide, where the selectable marker gene provides tolerance or resistance to the selection agent. Thus, the selection agent can bias or favor the survival, development, growth, proliferation, etc., of transformed cells expressing the selectable marker gene. Selectable marker genes may include, for example, those conferring tolerance or resistance to antibiotics, such as kanamycin and paromomycin (nptll), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (aroA or Cp4-EPSPS). Selectable marker genes which provide an ability to visually screen for transformants may also be used such as, for example, luciferase or green fluorescent protein (GFP), or a gene expressing a beta glucuronidase or uidA gene (GUS) for which various chromogenic substrates are known. In some embodiments, a nucleic acid molecule provided herein contains a selectable marker gene selected from the group consisting of nptll, aph IV, aadA, aac3, aacC4, bar, pat, DMO, EPSPS, aroA, luciferase, GFP, and GUS.

### Plants and Plant Cells

Certain aspects of the present disclosure relate to plants and plant cells that contain recombinant Cas12J polypeptides that are targeted to one or more target nucleic acids in the plant/plant cell in order to edit/modify the target nucleic acid.

As used herein, a “plant” refers to any of various photosynthetic, eukaryotic multi-cellular organisms of the kingdom Plantae, characteristically producing embryos, containing chloroplasts, having cellulose cell walls and lacking locomotion. As used herein, a “plant” includes any plant or part of a plant at any stage of development, including seeds, suspension cultures, plant cells, embryos, meristematic regions, callus tissue, leaves, roots, shoots, gametophytes, sporophytes, pollen, microspores, and progeny thereof. Also included are cuttings, and cell or tissue cultures. As used in conjunction with the present disclosure, plant tissue includes, for example, whole plants, plant cells, plant organs, e.g., leafs, stems, roots, meristems, plant seeds, protoplasts, callus, cell cultures, and any groups of plant cells organized into structural and/or functional units.

Various plant cells may be used in the present disclosure so long as they remain viable after being transformed or otherwise modified to express recombinant nucleic acids or house recombinant polypeptides. Preferably, the plant cell is not adversely affected by the transduction of the necessary nucleic acid sequences, the subsequent expression of the proteins or the resulting intermediates.

As disclosed herein, a broad range of plant types may be modified to incorporate recombinant polypeptides and/or polynucleotides of the present disclosure. Suitable plants that may be modified include both monocotyledonous (monocot) plants and dicotyledonous (dicot) plants.

Examples of suitable plants may include, for example, species of the Family Gramineae, including Sorghum bicolor and Zea mays; species of the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, and Triticum.

In some embodiments, plant cells may include, for example, those from corn (Zea mays), canola (Brassica napus, Brassica rapa ssp.), Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), duckweed (Lemna), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucijra), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia spp.), almond (Prunus amygdalus), sugar beets (Beta vulgaris), sugarcane (Saccharum spp.), oats, barley, vegetables, ornamentals, and conifers.

Examples of suitable vegetables plants may include, for example, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Examples of suitable ornamental plants may include, for example, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbiapulcherrima), and chrysanthemum.

Examples of suitable conifer plants may include, for example, loblolly pine (Pinus taeda), slash pine (Pinus elliotii), ponderosa pine (Pinus ponderosa), lodgepole pine (Pinus contorta), Monterey pine (Pinus radiata), Douglas-fir (Pseudotsuga menziesii), Western hemlock (Isuga canadensis), Sitka spruce (Picea glauca), redwood (Sequoia sempervirens), silver fir (Abies amabilis), balsam fir (Abies balsamea), Western red cedar (Thuja plicata), and Alaska yellow-cedar (Chamaecyparis nootkatensis).

Examples of suitable leguminous plants may include, for example, guar, locust bean, fenugreek, soybean, garden beans, cowpea, mungbean, lima bean, fava bean, lentils, chickpea, peanuts (Arachis sp.), crown vetch (Vicia sp.), hairy vetch, adzuki bean, lupine (Lupinus sp.), trifolium, common bean (Phaseolus sp.), field bean (Pisum sp.), clover (Melilotus sp.) Lotus, trefoil, lens, and false indigo.

Examples of suitable forage and turf grass may include, for example, alfalfa (Medicago s sp.), orchard grass, tall fescue, perennial ryegrass, creeping bent grass, and redtop.

Examples of suitable crop plants and model plants may include, for example, Arabidopsis, corn, rice, alfalfa, sunflower, canola, soybean, cotton, peanut, sorghum, wheat, tobacco, and lemna.

The plants and plant cells of the present disclosure may be genetically modified in that recombinant nucleic acids have been introduced into the plants, and as such the genetically modified plants and/or plant cells do not occur in nature. A suitable plant of the present disclosure is e.g. one capable of expressing one or more nucleic acid constructs encoding one or more recombinant proteins. The recombinant proteins encoded by the nucleic acids may be e.g. recombinant Cas12J polypeptides.

As used herein, the terms “transgenic plant” and “genetically modified plant” are used interchangeably and refer to a plant which contains within its genome a recombinant nucleic acid. Generally, the recombinant nucleic acid is stably integrated within the genome such that the polynucleotide is passed on to successive generations. However, in certain embodiments, the recombinant nucleic acid is transiently expressed in the plant. The recombinant nucleic acid may be integrated into the genome alone or as part of a recombinant expression cassette. “Transgenic” is used herein to include any cell, cell line, callus, tissue, plant part or plant, the genotype of which has been altered by the presence of exogenous nucleic acid including those transgenics initially so altered as well as those created by sexual crosses or asexual propagation from the initial transgenic.

Plant transformation protocols as well as protocols for introducing recombinant nucleic acids of the present disclosure into plants may vary depending on the type of plant or plant cell, e.g., monocot or dicot, targeted for transformation. Suitable methods of introducing recombinant nucleic acids of the present disclosure into plant cells and subsequent insertion into the plant genome include, for example, microinjection (Crossway et al., Biotechniques (1986) 4:320-334), electroporation (Riggs et al., Proc. Natl. Acad Sci. USA (1986) 83:5602-5606), Agrobacterium-mediated transformation (U.S. Pat. No. 5,563,055), direct gene transfer (Paszkowski et al., EMBO J. (1984) 3:2717-2722), and ballistic particle acceleration (U.S. Pat. No. 4,945,050; Tomes et al. (1995). “Direct DNA Transfer into Intact Plant Cells via Microprojectile Bombardment.” in Plant Cell, Tissue, and Organ Culture: Fundamental Methods, ed. Gamborg and Phillips (Springer-Verlag, Berlin); and McCabe et al., Biotechnology (1988) 6:923-926).

Additionally, recombinant polypeptides of the present disclosure can be targeted to a specific organelle within a plant cell. Targeting can be achieved by providing the recombinant protein with an appropriate targeting peptide sequence. Examples of such targeting peptides include, for example, secretory signal peptides (for secretion or cell wall or membrane targeting), plastid transit peptides, chloroplast transit peptides, mitochondrial target peptides, vacuole targeting peptides, nuclear targeting peptides, and the like (e.g., see Reiss et al., Mol. Gen. Genet. (1987) 209(1):116-121; Settles and Martienssen, Trends Cell Biol (1998) 12:494-501; Scott et al., J Biol Chem (2000) 10:1074; and Luque and Coreas, J Cell Sci (2000) 113:2485-2495).

Modified plant may be grown in accordance with conventional methods (e.g., see McCormick et al., Plant Cell. Reports (1986) 81-84.). These plants may then be grown, and pollinated with either the same transformed strain or different strains, with the resulting hybrid having the desired phenotypic characteristic. Two or more generations may be grown to ensure that the subject phenotypic characteristic is stably maintained and inherited and then seeds harvested to ensure the desired phenotype or other property has been achieved.

The present disclosure also provides plants derived from plants having an edited/modified nucleic acid as a consequence of the methods of the present disclosure. A plant having an edited/modified nucleic acid as a consequence of the methods of the present disclosure may be crossed with itself or with another plant to produce an F1 plant. In some embodiments, one or more of the resulting F1 plants may also have an edited/modified nucleic acid. Accordingly, in some embodiments, provided are progeny plants that are the progeny (either directly or indirectly) of plants having an edited/modified nucleic acid as a consequence of the methods of the present disclosure. These progeny plants may also have an edited/modified nucleic acid. Progeny plants may also have an altered or modified phenotype as compared to a corresponding control plant.

Further provided are methods of screening plants derived from plants having an edited/modified nucleic acid as a consequence of the methods of the present disclosure. In some embodiments, the derived plants (e.g. F1 or F2 plants resulting from or derived from crossing the plant having an edited/modified nucleic acid expression as a consequence of the methods of the present disclosure with another plant) can be selected from a population of derived plants. For example, provided are methods of selecting one or more of the derived plants that (i) lack recombinant nucleic acids, and (ii) have an edited/modified nucleic acid. Because the edit/modification of the target nucleic acid may be heritable, progeny plants as described herein do not necessarily need to contain a recombinant Cas12J polypeptide and/or a guide RNA in order to maintain the edit/modification to the target nucleic acid.

Plants with genetic backgrounds that are susceptible to transgene silencing may exhibit reduced Cas12J-mediated editing efficiency. It may thus be desirable, in some embodiments, to employ a genetic background that has reduced or eliminated susceptibility to transgene silencing. In some embodiments, employing a genetic background with reduced or eliminated susceptibility to transgene silencing may improve editing efficiency. Exemplary genetic backgrounds with reduced or eliminated susceptibility to transgene silencing will be readily apparent to one of skill in the art and include, for example, plants with mutations in RDR6 that reduce or eliminate RDR6 expression or function.

Conducting the methods of the present disclosure in a plant with a genetic background that reduces or eliminates susceptibility to transgene siliencing may increase the relative editing efficiency of a target nucleic acid by, for example, at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more as compared to a corresponding control (e.g. a wild-type plant).

### Methods of Modifying a Target Nucleic Acid

Growing and/or cultivation conditions sufficient for the recombinant polypeptides and/or polynucleotides of the present disclosure to be expressed and/or maintained in the plant/plant cell and to be targeted to and edit/modify one or more target nucleic acids of the present disclosure are well known in the art and include any suitable growing conditions disclosed herein. Typically, the plant is grown under conditions sufficient to express a recombinant polypeptide of the present disclosure, and for the expressed recombinant polypeptides to be localized to the nucleus of cells of the plant in order to be targeted to and edit/modify the target nucleic acids (if those target nucleic acids are present in the nucleus). Generally, the conditions sufficient for the expression of the recombinant polypeptide (if being encoded from a recombinant nucleic acid) will depend on the promoter used to control the expression of the recombinant polypeptide. For example, if an inducible promoter is utilized, expression of the recombinant polypeptide in a plant will require that the plant to be grown in the presence of the inducer.

Growth Conditions

As noted above, growing conditions sufficient for the recombinant polypeptides of the present disclosure to be expressed and/or maintained in the plant and to be targeted to one or more target nucleic acids to edit/modify the one or more target nucleic acids may vary depending on a number of factors (e.g. species of plant, use of inducible promoter, etc.). Suitable growing conditions may include, for example, ambient environmental conditions, standard laboratory conditions, standard greenhouse conditions, growth in long days under standard environmental conditions (e.g. 16 hours of light, 8 hours of dark), growth in 12 hour light: 12 hour dark day/night cycles, etc.

Plants and/or plant cells of the present disclosure housing a recombinant Cas12J polypeptide and a guide RNA may be maintained at a variety of temperatures. In general, the temperature should be sufficient for the Cas12J polypeptide and guide RNA to form, maintain, or otherwise be present as a complex that is able to target a target nucleic acid in order to edit/modify the target nucleic acids. Exemplary growth/cultivation temperatures include, for example, at least about 20° C., at least about 21° C., at least about 22° C., at least about 23° C., at least about 24° C., at least about 25° C., at least about 26° C., at least about 27° C., at least about 28° C., at least about 29° C. at least about 30° C. at least about 31° C., at least about 32° C., at least about 33° C., at least about 34° C., at least about 35° C., at least about 36° C., at least about 37° C., at least about 38° C., at least about 39° C., or at least about 40° C. Exemplary growth/cultivation temperatures include, for example, about 20° C. to about 25° C., about 25° C. to about 30° C. about 30° C. to about 35° C., or about 35° C. to about 40° (C. Plants and plant cells may be maintained at a constant temperature throughout the duration of the growth and/or incuation period, or the temperature schedule can be adjusted at various points throughout the duration of the growth and/or incuation period as will be readily apparent to one of skill in the art depending on the particular growth and/or incubation purpose.

In some embodiments, plants and plant cells may be maintained at a relative constant temperature with one or more periodic or intermittent exposures to a different temperature. For example, a plant or plant cell may be maintained at e.g. 20° C.-25° C. and then have a brief exposure to a different temperature (e.g. 37° C. for between 5 minutes to 5 hours), and then be returned to the original growth temperature (e.g. 20° C.-25° C.). The exposure to a different temperature may occur once or it may occur on a plurality of occasions over the full growth interval of plants and plant cells according to the methods of the present disclosure.

In some embodiments, plants and plant cells may be exposed to a first temperature and a second temperature for varying amounts of time, where the first and second temperatures are not the same temperature/are different temperatures. In some embodiments, the first temperature may be, for example, at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C. at least about 33° C., at least about 34° C., at least about 35° C., at least about 36° C., at least about 37° C., at least about 38° C., at least about 39° C., or at least about 40° C. and the duration of exposure to the first temperature may be, for example, about 30 minutes, about 45 minutes, about 1 hour, about 2.5 hours, about 5 hours, about 7.5 hours, about 10 hours, about 15 hours, about 20 hours, about 1 day, about 5 days, about 10 days, about 15 days, about 20 days, about 25 days, about 30 days, about 35 days, about 40 days, about 45 days, about 50 days, or about 55 days or mom. In some embodiments, the second temperature may be, for example, at least about 20° C., at least about 21° C. at least about 22° C., at least about 23° C., at least about 24° C., at least about 25° C., at least about 26° C., at least about 27° C., at least about 28° C., at least about 29° C., at least about 30° C., at least about 31° C. at least about 32° C., at least about 33° C., at least about 34° C. at least about 35° C. at least about 36° C., at least about 37° C., at least about 38° C., at least about 39° C. or at least about 40° C. and the duration of exposure to the second temperature may be, for example, about 30 minutes, about 45 minutes, about 1 hour, about 2.5 hours, about 5 hours, about 7.5 hours, about 10 hours, about 15 hours, about 20 hours, about 1 day, about 5 days, about 10 days, about 15 days, about 20 days, about 25 days, about 30 days, about 35 days, about 40 days, about 45 days, about 50 days, or about 55 days or more.

Various time frames may be used to observe editing/modification of a target nucleic acid according to the methods of the present disclosure. Plants and/or plant cells may be observed/assayed for editing/modification of a target nucleic acid after, for example, about 30 minutes, about 45 minutes, about 1 hour, about 2.5 hours, about 5 hours, about 7.5 hours, about 10 hours, about 15 hours, about 20 hours, about 1 day, about 5 days, about 10 days, about 15 days, about 20 days, about 25 days, about 30 days, about 35 days, about 40 days, about 45 days, about 50 days, or about 55 days or more after being cultivated/grown in conditions sufficient for a Cas12J polypeptide to facilitate editing/modification of a target nucleic acid.

Editing/Modifying a Target Nucleic Acid

Certain aspects of the present disclosure relate to editing or modifying a target nucleic acid using Cas12J polypeptides. In some embodiments, a Cas12J polypeptide is used to create a mutation in a target nucleic acid. Mutation of a nucleic acid generally refers to an insertion, deletion, substitution, duplication, or inversion of one or more nucleotides in the nucleic acid as compared to a reference or control nucleotide sequence.

In some embodiments, a Cas12J polypeptide of the present disclosure may induce a double-stranded break (DSB) at a target site of a nucleic acid sequence that is then repaired by the natural processes of either homologous recombination (HR) or non-homologous end-joining (NHEJ). Sequence modifications, such as for example insertions and deletions, can occur at the DSB locations via NHEJ repair. If two DSBs flanking one target region are created, the breaks can be repaired via NHEJ by reversing the orientation of the targeted DNA (also referred to as an “inversion”). HR can be used to integrate a donor nucleic acid sequence into a target site. In one aspect, a double-stranded break provided herein is repaired by NHEJ. In another aspect, a double-stranded break provided herein is repaired by HR.

In some embodiments, a Cas12J polypeptide of the present disclosure may induce a double-stranded break with 5′ nucleotide overhangs at a target site of a nucleic acid sequence such that an exogenous DNA segment of interest can serve as the donor nucleic acid to be ligated into the target nucleic acid. The presence of 5′ nucleotide overhangs allows the insertion of the exogenous DNA to be directional.

In some embodiments, a nucleic acid that encodes a polypeptide may be targeted and edited such that the modification to the nucleic acid results in a change to one or more codons in the encoded polypeptide. In some embodiments, the modification of the target nucleic acid may result in deletion of one or more codons in the encoded polypeptide.

A target nucleic acid of the present disclosure may be edited or modified in a variety of ways (e.g. deletion of nucleotides in the target nucleic acid) depending on the particular application as will be readily apparent to one of skill in the art. A target nucleic acid subjected to the methods of the present disclosure may have an edit or modification of at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, or at least 25 nucleotides or more.

A target nucleic acid of the present disclosure may have its expression decreased/downregulated as compared to a corresponding control nucleic acid. A target nucleic acid of the present disclosure in a plant cell housing recombinant polypeptides of the present disclosure may have its expression decreased/downregulated by at least about 5%, at least about 10%, at least about 15%, at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% as compared to a corresponding control. Various controls will be readily apparent to one of skill in the art. For example, a control may be a corresponding plant or plant cell that does not contain recombinant polypeptides of the present disclosure (e.g. wild-type plant or plant cell).

A target nucleic acid may have its expression decreased/downregulated at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 40-fold, at least about 50-fold, at least about 75-fold, at least about 100-fold, at least about 150-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 1,250-fold, at least about 1,500-fold, at least about 1,750-fold, at least about 2,000-fold, at least about 2,500-fold, at least about 3,000-fold, at least about 3.500-fold, at least about 4,000-fold, at least about 4,500-fold, at least about 5,000-fold, at least about 5,500-fold, at least about 6.000-fold, at least about 6,500-fold, at least about 7,000-fold, at least about 7,500-fold, at least about 8.000-fold, at least about 8,500-fold, at least about 9,000-fold, at least about 9,500-fold, at least about 10.000-fold, at least about 12,000-fold, at least about 14,00-fold, at least about 16,000-fold, at least about 18,000-fold, or at least about 20.000-fold or more as compared to a corresponding control nucleic acid. As stated above, various controls will be readily apparent to one of skill in the art. For example, a control nucleic acid may be a corresponding nucleic acid from a plant or plant cell that does not contain a nucleic acid encoding a recombinant polypeptide of the present disclosure.

A target nucleic acid of the present disclosure may have its expression increased/upregulated/activated as compared to a corresponding control nucleic acid. A target nucleic acid of the present disclosure in a plant cell housing recombinant polypeptides of the present disclosure may have its expression increased/upregulated/activated by at least about 5%, at least about 10%, at least about 15%, at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% as compared to a corresponding control. Various controls will be readily apparent to one of skill in the art. For example, a control may be a corresponding plant or plant cell that does not contain recombinant polypeptides of the present disclosure (e.g. wild-type plant or plant cell).

A target nucleic acid may have its expression increased/upregulated/activated at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 40-fold, at least about 50-fold, at least about 75-fold, at least about 100-fold, at least about 150-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 1,250-fold, at least about 1,500-fold, at least about 1,750-fold, at least about 2,000-fold, at least about 2,500-fold, at least about 3,000-fold, at least about 3,500-fold, at least about 4,000-fold, at least about 4,500-fold, at least about 5,000-fold, at least about 5,500-fold, at least about 6,000-fold, at least about 6,500-fold, at least about 7.000-fold, at least about 7,500-fold, at least about 8,000-fold, at least about 8,500-fold, at least about 9,000-fold, at least about 9.500-fold, at least about 10,000-fold, at least about 12,000-fold, at least about 14,00-fold, at least about 16,000-fold, at least about 18.000-fold, or at least about 20,000-fold or more as compared to a corresponding control nucleic acid. As stated above, various controls will be readily apparent to one of skill in the art. For example, a control nucleic acid may be a corresponding nucleic acid from a plant or plant cell that does not contain a nucleic acid encoding a recombinant polypeptide of the present disclosure.

Certain aspects of the present disclosure relate to increasing editing efficiency of CAS12J polypeptides of the present disclosure. Editing frequency and efficiency, as well as methods of determining such, are well-known in the art. Generally speaking, editing efficiency is evaluated by determining the observed quantity of a given target sequence that experienced an editing event (editing frequency) as compared to the total quantity of the target sequence observed (whether edited or unedited). An increase in editing efficiency generally refers to an increase in the number of sequences experiencing an editing event (editing frequency) as compared to the total quantity of the target sequence observed (whether edited or unedited).

In some embodiments, increases in editing efficiency are compared to corresponding controls in relative terms (relative editing efficiency). For example, if the absolute editing frequency in one condition is 0.5% and the absolute editing frequency in a second condition is 1%, the second condition represents a doubling of the absolute editing frequency relative to the first condition, or in other words, the second condition represents a 100% increase in relative editing efficiency as compared to the first condition.

The frequency or efficiency of editing of a target nucleic acid of the present disclosure may vary. For example, the particular promoter used to drive gRNA expression may influence the editing efficiency of a target nucleic acid. In some embodiments, use of a Pol II promoter (e.g. a CmYLCV promoter) to drive gRNA expression may result in increased editing efficiency as compared to a corresponding control promoter (e.g. a Pol III promoter, such as a U6 promoter for example). Use of a Pol II promoter to drive gRNA expression may increase the relative editing efficiency of a target nucleic acid by, for example, at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more as compared to a corresponding control (e.g. a U6 promoter).

Various conditions or variables described herein may improve editing efficiency of a Cas12J polypeptide as described herein (e.g. targeting a region of open chromatin for editing, use of a ribozyme in the gRNA targeting, performing editing in a plant genetic background that exhibits reduced transgene silencing, etc.) as compared to corresponding control conditions or variables. Various conditions or variables described herein may increase the relative editing efficiency of a target nucleic acid by, for example, at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 125%, at least about 150%, at least about 175%, at least about 200%, at least about 225%, at least about 250%, at least about 275%, or at least about 300% or more as compared to a corresponding control condition or variable. Applicable control conditions or variables will be readily apparent to one of skill in the art depending on the particular editing context. For example, the corresponding control may be as compared to a region of closed chromatin or heterochromatin, editing without the use of a ribozyme, and/or editing in a plant genetic background that exhibits relatively high transgene silencing.

Comparisons in the present disclosure may also be in reference to corresponding control plants/plant cells. Various control plants will be readily apparent to one of skill in the art. For example, a control plant or plant cell may be a plant or plant cell that does not contain one or more of: (1) a recombinant Cas12J polypeptide, (2) a guide RNA, and/or (3) both a recombinant Cas12J polypeptide and a guide RNA.

Methods of probing the expression level of a nucleic acid are well-known to those of skill in the art. For example, qRT-PCR analysis may be used to determine the expression level of a population of nucleic acids isolated from a nucleic acid-containing sample (e.g. plants, plant tissues, or plant cells).

### Kits

Certain aspects of the present disclosure relate to an article of manufacture or kit comprising a polynucleotide, vector, cell, and/or composition described herein. In some embodiments, the kit further comprises a packed insert comprising instructions for the use of the polynucleotide, vector, cell, and/or composition. In some embodiments, the article of manufacture or kit further comprises one or more buffer, e.g., for storing, transferring, or otherwise using the polynucleotide, vector, cell, and/or composition. In some embodiments, the kit further comprises one or more containers for storing the polynucleotide, vector, cell, and/or composition.

The foregoing written description is considered to be sufficient to enable one skilled in the art to practice the present disclosure. The following Examples are offered for illustrative purposes only, and are not intended to limit the scope of the present disclosure in any way. Indeed, various modifications of the present disclosure in addition to those shown and described herein will become apparent to those skilled in the art from the foregoing description and fall within the scope of the appended claims.

### EXAMPLES

The following examples are offered to illustrate provided embodiments and are not intended to limit the scope of the present disclosure. In the Examples provided herein, tables appear beneath the table heading that describes the respective table.

### Example 1: CAS12J-2 Conducts Gene Editing in Plant Cells

This Example demonstrates that CAS12J-2, as a member of the most minimal functional CRISPR-Cas system ever discovered, is able to conduct gene editing in plant cells. The in vivo gene editing in plant cells can be achieved by introducing DNA into cells which encodes the CAS12J-2 protein and the corresponding CAS12J-2 guide RNA for a target of interest, or by introducing RNPs into cells which are composed of CAS12J-2 proteins already loaded with guide RNA. CAS12J-2 is able to edit a target gene in a standard 23° C. environment and in a 23° C. environment with a 37° C. incubation period added, displaying a wide suitable temperature range which allows application of CAS12J-2 on a wide variety of organisms including plants and cold-blooded animals with lower body temperature.

### Introduction

Traditional CAS proteins used in CRISPR-based targeting systems (e.g. Cas9 and Cpf1) are derived from gut bacteria and therefore evolved in a high temperature optimum (e.g. 37° C.). However, this high temperature is not ideal or practical for many plant species and therefore creates challenges for creating practical CRISPR targeting systems in plants and other eukaryotic organisms. Indeed, evidence showing that heat shocks to plants can allow for stronger gene editing supports the idea that existing CRISPR proteins (e.g. Cas9 and Cpf1) are not ideal for use in plants (PMID: 29161464, PMID: 30950179, PMID: 30704461, PMID: 29972722). Exploring whether other RNA-guided nuclease proteins are better suited for use in CRISPR-based targeting systems in plants is therefore warranted.

To investigate whether CAS12J-2 is able to conduct targeted gene editing in plant systems, mesophyll protoplasts were isolated from Arabidopsis leaves and the CAS12J-2 editing components were introduced to these protoplasts via PEG-CaCl₂ transfection. AtPDS3 was chosen as the target gene due to the fact that (1) previous data suggests it has an accessible chromatin state, and (2) Arabidopsis mutant plants of AtPDS3 gene show white color which should allow for easy scoring of CAS12J-2 edited transgenic plants. The AtPDS3 gene sequence is listed as SEQ ID NO: 11 (coding sequences highlighted in bold), with the coding sequences also shown separately as SEQ ID NO: 12. 10 guide RNAs for CAS12J-2 targeting AtPDS3 coding region were designed based on the PAM sequence of CAS12J-2 (See Table 1-1).

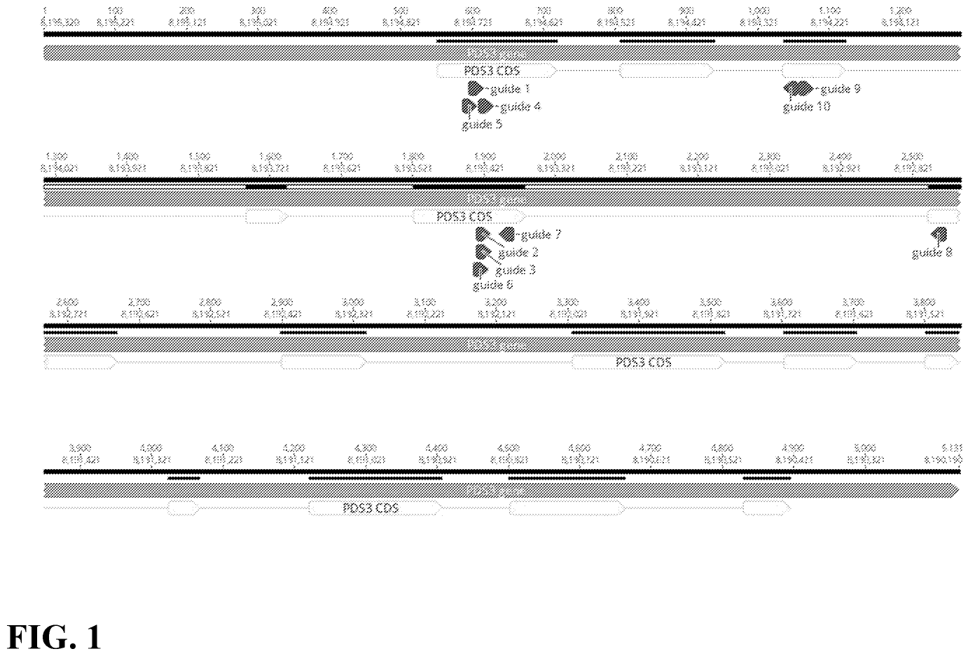

Two methods were used to introduce CAS12J-2 editing components into protoplasts: (1) transfection of plasmid DNA which contains CAS12J-2 expression cassette and CAS12J-2 guide RNA transcription cassette; and (2) transfection of CAS12J-2 RNPs which already have CAS12J-2 guide RNA bound to CAS12J-2 protein. 10 different guide RNAs targeting different regions of the AtPDS3 gene were tested (See FIG. 1 and Table 1-1).

### Materials and Methods

Plasmid Construction

Plasmid construction proceeded in three Steps, defined below as Step 1, Step 2, and Step 3. Step3 further has 3 sub-steps, defined below as Step 3-1, Step 3-2, and Step 3-3.

Step1: CAS12J-2-2×SV40NLS-2×FLAG coding sequence (without IV2 intron) was codon optimized and synthesized by IDT. For both version1 and version2 plasmids, the CAS12J coding portion (CAS12J, IV2 intron, NLS, FLAG) was first assembled in HBT vector backbone with the following method:

For version 1, the HBT-pcoCAS9 vector (addgene52254) backbone (including 35sPPDK promoter, N-ter2×FLAG-SV40NLS and Nos terminator) was amplified by PCR. The IV2 intron was also amplified from the HBT-pcoCAS9 vector, with >=16 bp overlapping sequence with CAS12J-2 coding sequence at the site for IV2 intron insertion. The Arabidopsis codon-optimized CAS12J-2 coding sequence was amplified using synthesized gene fragment from IDT as the template, and amplified as two PCR fragments, separated at the site of IV2 intron insertion, both with >=16 bp overlapping sequences with the corresponding side of the HBT-pcoCAS9 backbone. The size of these four PCR fragments were checked by gel electrophoresis. The fragments were then purified, and assembled together using the TAKARA in-fusion HD cloning kit (cat639650). The sequence of the resulting HBT-pcoCAS12J-2 version1 plasmid was checked by Sanger sequencing.

For version 2, the HBT-pcoCAS9 vector (addgene52254) backbone (including 35sPPDK promoter and Nos terminator) was amplified by PCR from HBT-pcoCAS9 vector. The IV2 intron was also amplified from the HBT-pcoCAS9 vector, with >=16 bp overlapping sequence with the CAS12J-2 coding sequence at the site for IV2 intron insertion. The Arabidopsis codon-optimized CAS12J-2 coding sequence, including the C-terminal 2×SV40NLS-2×FLAG coding sequence, was amplified using synthesized gene fragments from IDT as templates, and amplified as two PCR fragments, separated at the site of IV2 intron insertion, both with >=16 bp overlapping sequences with the corresponding side of the HBT-pcoCAS9 backbone. The size of these four PCR fragments were checked by gel electrophoresis. The fragments were then purified, and assembled together using the TAKARA in-fusion HD cloning kit (cat639650). The sequence of the resulting HBT-pcoCAS12J-2 version2 plasmid was checked by Sanger sequencing.

Step 2: The binary vectors of pCAMBIA1300_pUB10.pcoCAS12J2_E9t_version1 MCS and pCAMBIA1300_pUB10_pcoCAS12J2_E9t_version2 MCS were constructed. These two binary vectors have the CAS12J-2 protein expression cassette with corresponding NLS and FLAG tag, driven by the promoter of the UBQ10 gene, and with the rbcS-E9 terminator at the end of the cassette. At this step, the guide RNA cassette has not been added yet. To construct these two plasmids, the following four fragments were assembled in an in-fusion reaction with the TAKARA in-fusion HD cloning kit: (1) pCAMBIA1300-pYAO-cas9 vector (named pYAO:hSpCas9 in PMID: 26524930) was digested with KpnI and EcoRI, and the larger fragment was gel purified; (2) the UBQ10 promoter; and (3) the rbcS-E9 terminator, amplified by PCR using a template vector containing these features. During PCR, >=16 bp of sequence was added by the primer to overlap with the pCAMBIA1300-pYAO-cas9 vector backbone fragment and with the coding sequence of CAS12J-2 protein with NLS and FLAG in version1 or version2 on the corresponding side of fragment end; (4) the coding sequences of CAS12J-2 protein with NLS and FLAG in version1 and version2 were amplified using the plasmid constructed in step 1 as the template. After the assembly of these four fragments for both version1 and version2 plasmids, Sanger sequencing was used to check the sequences.

The Cas12J-2 expression cassette with the amino acid sequence of CAS12J-2 with NLS and FLAG tag in version 1 is presented in SEQ ID NO: 17. In SEQ ID NO: 17, bold letters indicate CAS12J-2 amino acids, italic letters indicate FLAG tag amino acids, and bold and italic letters indicate NLS amino acids. The amino acid sequence of a single FLAG tag is presented in SEQ ID NO: 18. The amino acid sequences of NLS sequences are presented in SEQ ID NO: 19 and SEQ ID NO: 20.

The Cas12J-2 expression cassette with the amino acid sequence of CAS12J-2 with NLS and FLAG tag in version 2 is presented in SEQ ID NO: 21. In SEQ ID NO: 21, bold letters indicate CAS12J-2 amino acids, italic letters indicate FLAG tag amino acids, and bold and italic letters indicate NLS amino acids.

Step 3: Clone the AtU6-26 guide RNA cassette into the plasmids from step 2.

Step 3-1: First, the pUC119-gRNA vector (addgene 52255) was used as a temporary vector for assembly of the CAS12J-repeat and the CAS12J-AtPDS3 guide RNAI spacer. The backbone of the vector, including the AtU6-1 promoter, was amplified with primer and purified by gel electrophoresis. The CAS12J-repeat and CAS12J-AtPDS3 guide RNAI spacer as well as poly-T terminator combined fragment were created by PCR with two long primers with 21 bp on the 3′ end complementary with each other, and with the 5′ sequences overlapping>=16 bp with the vector backbone. No other templates were used in this PCR reaction. The vector fragment and the gRNA fragment were assembled using the TAKARA in-fusion HD cloning kit.

Step 3-2: The products of step 2, which are the pCAMBIA1300_pUB10_pcoCAS12J2_E9t_version1 MCS and pCAMBIA1300_pUB10_.pcoCAS12J2_E9t_version2 MCS plasmids, were opened by digestion with SpeI (step 3-2 backbone). The AtU6-26 promoter, which is slightly more efficient than the AtU6-1 promoter, was amplified from a template construct containing this feature, with >=16 bp overlapping with the step3-2 backbone on the corresponding side (step 3-2 fragment1). A poly-T terminator and a fragment of DNA sequence on pCAMB1300_pYaocas9_RING2_gRNA1 downstream of the gRNA cassette poly-T terminator were amplified with >=16 bp overlapping with the step 3-2 backbone on the corresponding side (step 3-2 fragment 2). The CAS12J-repeat-AtPDS3 guide RNAI spacer-poly-T terminator fragment was amplified from the plasmid generated in step 3-1, with >=16 bp overlapping with step 3-2 fragment 1 and step 3-2 fragment 2 on the corresponding sides. Then, these four fragments were assembled together with the TAKARA in-fusion HD cloning kit. Sanger sequencing was used to check the product sequence. The products of step 3-2 were termed pCAMBIA1300_pUB10_pcoCAS12J2_E9t_version1_AtPDS3_gRNA1, and pCAMBIA1300_pUB10_pcoCAS12J2_E9t_version2_AtPDS3_gRNA1, for version1 and version2, respectively.

Step3-3: This step served to clone other AtPDS3 guide RNAs into the binary vector with the CAS12J-2 protein expression cassette (product of step 2), for each AtPDS3 guide RNA, using the product plasmids of step 3-2 as template. First, the AtU6-26promoter-CAS12J_repeat was amplified to have >=16 bp overlapping sequence with the step 3-2 backbone on the upstream end, and the AtPDS3 guide RNA spacer sequence of interest (20 bp—See Table 1-1) was added by primer on the downstream end. Then, the poly-T terminator and an 82 bp DNA sequence after the poly-T terminator were amplified to have the AtPDS3 guide RNA spacer sequence of interest (20 bp—See Table 1-1) on the upstream end, added by primer, and >=16 bp overlapping sequence with the step 3-2 backbone on the downstream end. The step 3-2 backbone and these two PCR fragments were assembled using the TAKARA in-fusion HD cloning kit. The resulting plasmids were checked with Sanger sequencing, and were termed the the pCAMBIA1300_pUB10_pcoCAS12J2_E9t_version1_AtPDS3_gRNA(1 to 10) and pCAMBIA1300_pUB10_pcoCAS12J2_E9t_version2_AtPDS3_gRNA(1 to 10) plasmids.

Table 1-1 depicts the guide RNA sequences used in plant plasmid vectors and RNPs. In both plant plasmid vectors and RNPs, guide RNAs are composed of two parts: a repeat and a spacer, with the spacer at the 3′ side of the repeat. Longer repeats and 20nt spacers were used in the plasmid vectors. In RNPs, a 25nt repeat with the same sequence as the later part of the repeat used for plasmids was used. In RNPs, the spacer sequences used were the first 18nt of spacer sequences for plasmids.

TABLE 1-1
Guide RNA sequence as used in plant
plasmid vectors and RNPs

| | CAS12J-2 Guide RNA repeat | | |
|---|---|---|---|
| Purpose | sequence (common to all guides) | | |
| For plasmid | GTCGGAACGCTCAACGATTGCCCCTC | | |
| vectors | ACGAGGGGAC (SEQ ID NO: 91) | | |
| For RNPs | CAACGATTGCCCCTCACGAGGGGAC | | |
| | (SEQ ID NO: 92) | | |
| | | | Direction |
| | Guide RNA spacer | | relative |
| | sequence | | to |
| APDS3 | (denoted in DNA | | AtPDS3 |
| Guide # | sequence) | PAM | gene |
| 1 | AAACGGGTTTTTGGAGGCAC | TCN | forward |
| | (SEQ ID NO: 93) | | |
| 2 | CTATGCCAAGTAAACCTGGA | TTN | forward |
| | (SEQ ID NO: 94) | | |
| 3 | TATGCCAAGTAAACCTGGAG | TGN | forward |
| | (SEQ ID NO: 95) | | |
| 4 | AGGCACTTTCATCTGGAGGT | TGN | forward |
| | (SEQ ID NO: 96) | | |
| 5 | GCCTTATCAAAACGGGTTT | TTN | forward |
| | (SEQ ID NO: 97) | | |
| 6 | TTGCTATGCCAAGTAAACCT | TTN | forward |
| | (SEQ ID NO: 98) | | |
| 7 | TAGGACATCTGGGAAGTCAA | TGN | reverse |
| | (SEQ ID NO: 99) | | |
| 8 | TTGTTCCGCAAAATAGCCCA | TCN | reverse |
| | (SEQ ID NO: 100) | | |
| 9 | AAAGTACCTGGCTGATGCAG | TGN | forward |
| | (SEQ ID NO: 101) | | |
| 10 | CAGTTGACAATCCAGCCAAT | TTN | reverse |
| | (SEQ ID NO: 102) | | |
| scramble | GCGACACGACTCATTATAAC | NONE | |
| control | (SEQ ID NO: 103) | | |

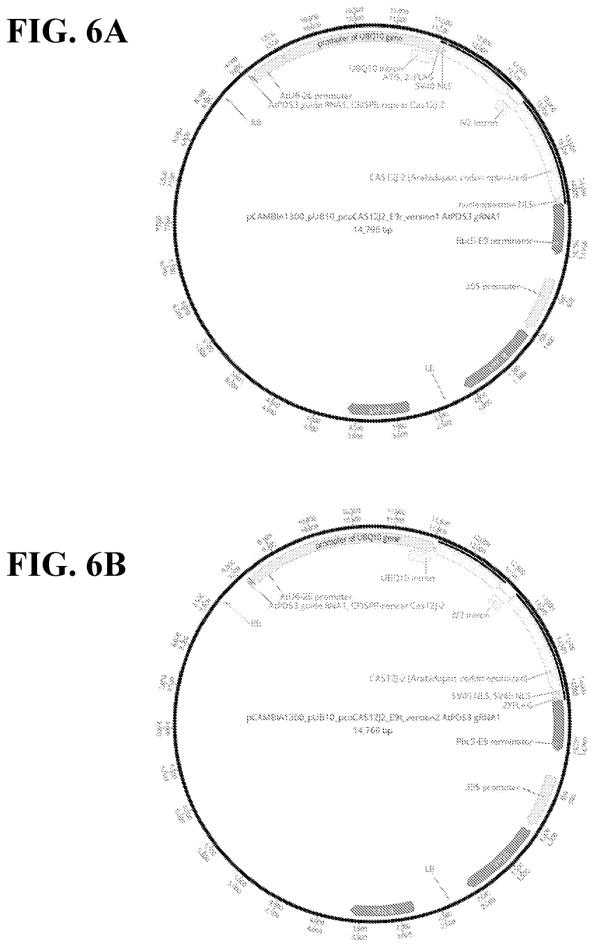

The maps of the resulting final plasmids are shown in FIG. 6A-6B. The corresponding plasmid sequences are shown in SEQ ID NO: 13 (version 1) and SEQ ID NO: 14 (version 2), with the AtPDS3 gRNA1 plasmids as an example. For SEQ ID NO: 13 and SEQ ID NO: 14, bold letters indicate CAS12J-2 DNA sequence (Arabidopsis codon optimized); italicized letters indicate the IV2 intron which is also listed as SEQ ID NO: 15; letters in bold and italic indicate guide RNA sequence (spacer part); and underlined letters indicate the CAS12J repeat sequence which is also listed as SEQ ID NO: 16.

For other AtPDS3 guides, the sequences are changed only for the spacer part according to Table 1-1. The corresponding plasmid sequences for other guides (AtPDS3 gRNA1 to AtPDS3 gRNA9) are only changed in the spacer sequence portion according to Table 1-1. Note that the guide RNA cassette is in the reverse direction compared to the CAS12J protein encoding cassette, such that the guide RNA sequence (depicted as DNA sequence) appear as reverse complements in the plasmid sequences.

Without wishing to be bound by theory, future experiments could involve constructing similar binary vectors with CAS12J-2 protein expression driven by the pYAO promoter, which is especially active in actively dividing cells. These constructs could be used to generate transgenic plants for examining CAS12J-2 function in whole plant organisms and to examine heritability patterns of mutant alleles created by CAS12J-2 editing. The nucleotide sequence of the pYAO promoter is presented in SEQ ID NO: 22.

RNP Reconstitution

Guide RNAs were synthesized (25nt repeat+18nt spacer as shown in Table 1-1) by Synthego. 5 nmol of dry RNA was dissolved by adding 10 μL of DEPC-treated H₂O. 5 μL of the dissolved RNA was incubated at 65° C. for 3 minutes, then cooled to room temperature. For RNP reconstitution, 3 μL of heated-and-cooled RNA was added to 292.2 μL 2×CB buffer (2×CB buffer contains: 20 mM Hepes-Na, 300 mM KCl, 10 mM MgCl₂, 20% glycrol, 1 mM TCEP; pH 7.5), vortexed to mix, and spun. Then, 4.8 μL of 250 μM CAS12J-2 protein was added and pipetted to mix. The mixture was then incubated at room temperature for 30 minutes. The resulting mixture contains 4 μM RNP in 2×CB buffer. All reagents were maintained as RNase free.

In Vitro RNP Cleavage Assay

The AtPDS3 gene fragments, which span all guide RNAs, were amplified by PCR. PCR products were run on gels to check for size (2.76 Kb) and gel extracted. The gel-extracted substrate was combined with RNP in a 1:100 molar ratio (substrate/Cas12J) in 1×CB, and the reaction was mixed by pipetting. The reaction was incubated at 37° C. for 1 hour, then stopped by addition of 50 μM EDTA. 1 μl of proteinase K (Invitrogen, 20 mg/μL) was added to the reaction and incubated for 20 minutes at 37° C. Then the reaction was run on 2% agarose gel for visualization.

Protoplast Isolation and Transfection

Protoplast isolation was performed as described in the following publication: PMID: 17585298. Special care was performed for an overall sterile environment when preparing protoplast.

For plasmids, protoplast transfection was performed by adding 20 μL of maxiprep plasmid (concentration between 0.92 μg/μL to 2.56 μg/μL for this Example) to 200 μL protoplast at 2×10⁵ cells/mL. The plasmids and cells were mixed by gently tapping the tube 3-4 times. Then 220 μL of fresh and sterile PEG-CaCl₂ solution (PMID: 17585298) were added to the protoplast-plasmid mixture and mixed well by gently tapping tubes. The protoplasts with PEG were incubated at room temperature for 10 minutes, then 880 μL of W5 solution (PMID: 17585298) was added and mixed with the protoplasts by inverting the tube 2-3 times to stop the transfection. Protoplasts were harvested by centrifugation at 100 rcf for 2 minutes, resuspended in 1 mL of WI, and plated into 6-well plates pre-coated with 5% calf serum. The lids of the 6-well plates were closed to begin the incubation of the protoplasts. For the 23-degree set, the protoplasts were incubated at 23° C. for 48 hours. For 28-degree set, the protoplasts were incubated at 28° C. in a plant incubator for 48 hours. For the 37-degree set, the protoplasts were incubated first at 23° C. for 20 hours, then moved to 37° C. for 2 hours. Then, the protoplasts were moved back to 23° C. and incubated for a total duration of 48 hours.

For RNPs, 26 μL of 4 μM RNP were first added to a round-bottom 2 mL tube. Then 200 μL of protoplasts (at 2×10⁵ cells/mL) were added to the tube. 2 μL of 5 μg/μL salmon sperm DNA was added and mixed gently by tapping the tube 3-4 times. Then, 228 μL of fresh, sterile and RNase free PEG-CaCl₂) solution (PMID: 17585298) was added to the protoplast-plasmid mixture and mixed well by gently tapping tubes. The protoplasts with PEG solution were incubated at room temperature for 10 minutes, then 880 μL of W5 solution (PMID: 17585298) was added and mixed with the protoplasts by inverting the tube 2-3 times to stop the transfection. Protoplasts were harvested by centrifugation at 100 ref for 2 min, resuspended in 1 mL WI, and plated into 6-well plates pre-coated with 5% calf serum. The lids of the 6-well plates were closed to begin the incubation of the protoplasts. For the 23-degree set, the protoplasts were incubated at 23° C. for 36 hours. For 37-degree set, protoplasts were incubated first at 23° C. for 12 hours, then moved to 37° C. for 2.5 hours. Then, the protoplasts were moved back to 23° C. and incubated for a total duration of 36 hours.

At the end of the incubations, the protoplasts were harvested by first centrifugation at 100 rcf for 2-3 minutes. Keeping the pellet, the supernatant was moved to another tube and went through another centrifugation at 3000 rcf for 3 minutes to collect any residue protoplasts. Pellets from these two centrifugations were combined and flash frozen for further analysis.

Amplicon Sequencing

DNAs of protoplast samples were extracted using the Qiagen DNeasy plant mini kit. Amplicons were obtained by two rounds of PCR. Amplification primers for the first round of PCR were designed to have the 3′ part of primer with sequences flanking a 200-300 bp fragment of the AtPDS3 gene around the guide RNA of interest. The 5′ part of the primer contained sequences to be bound by common sequencing primers (for reading paired-end reads, read 1 and read 2). The primers were designed so that the gRNA sequence started from within 100 bp from the beginning of read 1. The first round of PCR was done with Thermo fusion enzyme. Half of all DNA from a protoplast sample was used as the template, and 25 cycles of amplification were done for the first round. Then the reaction was cleaned by 1× Ampure XP beads. The elution from the cleanup was used as the template for the second round of PCR by fusion enzyme with 12 cycles. The second round of PCR was designed so that indexes were added to each sample. The samples were then purified by 0.8-1× Ampure beads for 1-2 rounds until no primer dimers were seen, with fragments below 200 bp considered primer dimers. Then amplicons were sent for paired-end 150 bp next generation sequencing.

Amplicon Sequencing Result Analysis

Reads were first quality- and adaptor-trimmed with trim-galore, then mapped to the AtPDS3 genomic region by BWA aligner. Sorted and indexed bam files were used as input files for further analysis by the CrispRvariants R package. Each mutation pattern with corresponding reads counts were exported by the CrispRvariants R package. After assessing all control samples, a criterion to classify reads containing deletions was established: only reads with >=3 bp deletion of same pattern (deletion of same size starting with same location) with >=100 reads counts from a sample were counted into the reads number with deletion. This criterion was established due to the fact that 1-bp indels and occasionally 2 bp deletions were observed with reads number>100 in control samples. Larger deletions were also observed at very low frequencies (much lower than 100 reads) in control samples. These observations indicate that occasional PCR inaccuracy and low-quality sequencing in a small fraction of reads can result in the deletion patterns with corresponding read number ranges as stated above in control samples. These stringent criteria were employed so that the counted deletion signals were true signal indicating editing events, though it is possible that CAS12J-2 might be able to create 1-2 bp indels at lower frequency.

### Results

In Vitro Cleavage Assay

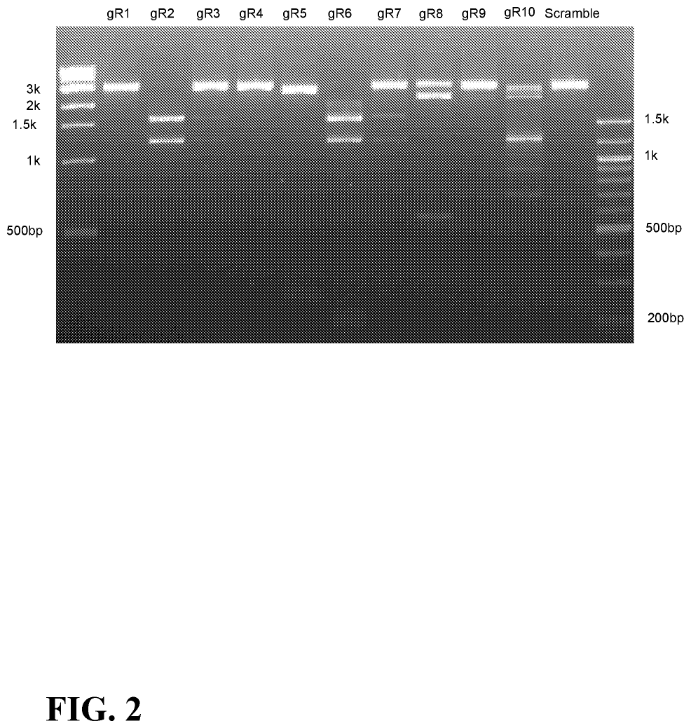

In an in vitro cleavage assay, CAS12J-2 RNPs with guide RNA 2, 5, 6 or 10 showed complete cleavage of target AtPDS3 gene fragment by 1-hour incubation at 37° C. RNPs with some other guides, such as gRNA 8, showed partial digestion of the substrate (FIG. 2).

Protein Expression

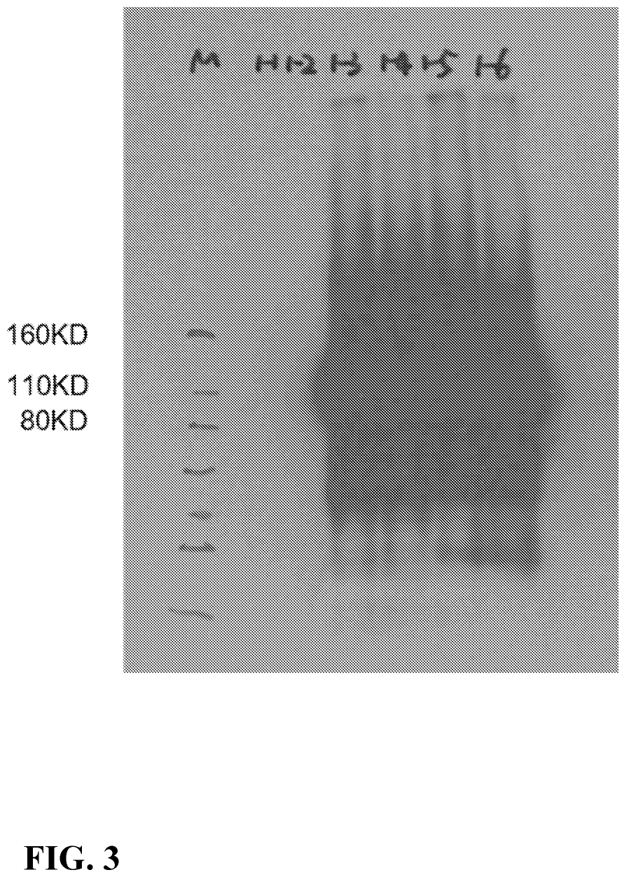

For plasmid transfection, two versions of plasmids were used, with the major difference being the format of fusing the nuclear localization signal (NLS) and flag tag to the CAS12J-2 protein (for which the Arabidopsis codon-optimized DNA sequence was used). In version 1 (ver1), 2× flag tag and one SV40 NLS was fused to the N-terminal end of CAS12J-2, and a nucleoplasmin NLS was fused to the C-terminal end of CAS12J-2. In version 2 (ver2), two SV40 NLS and 2× flag tag were fused to the C-terminal end of CAS12J-2. In both versions, an IV2 intron (modified second intron of the potato ST-LSI gene) was inserted into the CAS12J-2 coding sequence for the purpose of enhancing the CAS12J-2 expression level in plants and preserving plasmid stability when culturing bacteria for plasmid extraction. Both versions of plasmids for gRNA 1, 2, 3, 4, 5 were tested. RNPs of gRNA 1 to 10 were also tested. Abundant CAS12J-2 protein expression was observed by western blot from both versions of plasmids (FIG. 3).

Gene Editing

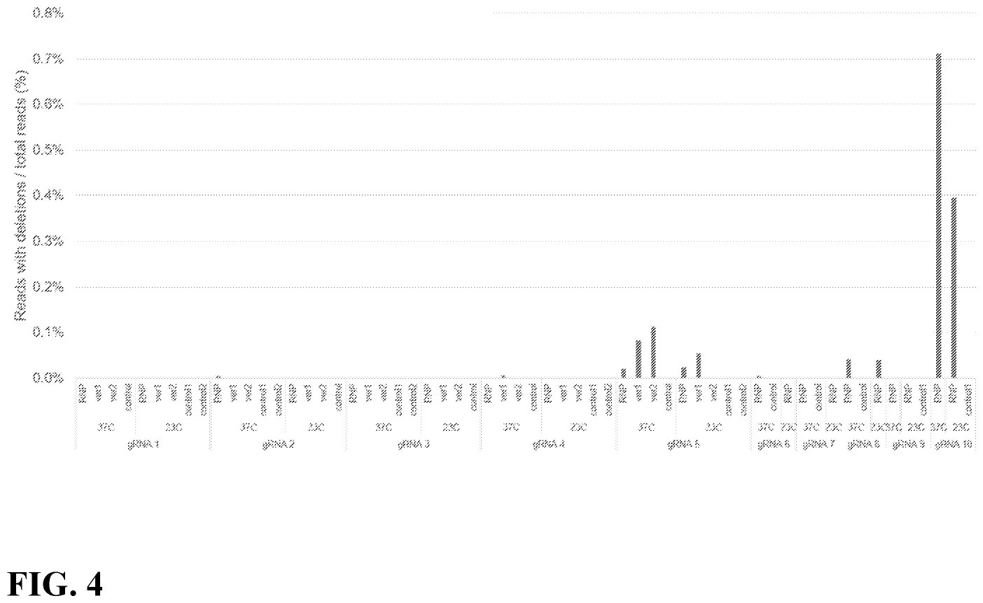

Successful gene editing events were detected for gRNA 5 with both the plasmid transfection (both versions of plasmid) and the RNP transfection (FIG. 4). RNP transfections also resulted in gene editing by gRNA8 and gRNA 10 (FIG. 4). Gene editing was detected by incubating transfected protoplasts at 23° C., or with 37° C. incubation added in the middle of 23° C. incubation (FIG. 4). Another set of plasmid protoplast transfection experiments were also performed for gRNA1 to gRNA5 with protoplasts being incubated at 28° C. Editing was also observed for gRNA5 with this set of experiments.

Editing Patterns

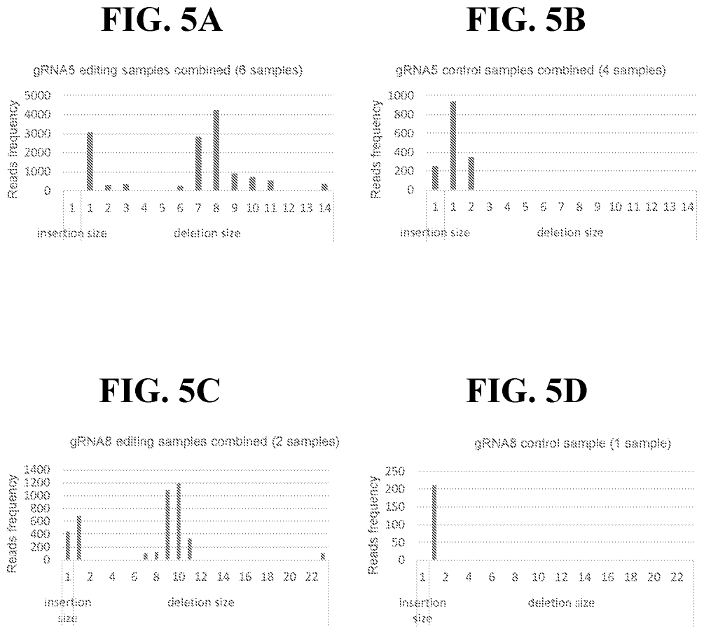

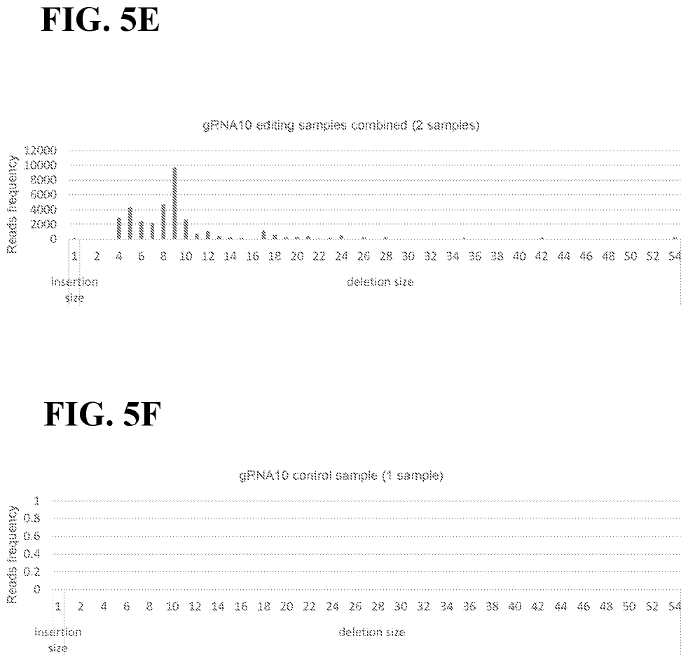

The in vivo editing by CAS12J-2 in plant cells preferably results in deletions with more than 3 bp. Detailed editing patterns detected from 3 example samples are shown in Table 1-2, Table 1-3, and Table 1-4. The highest deletion frequency appears to be around 8-10 bp (FIG. 5A-FIG. 5F). Without wishing to be bound by theory, it is possible that CAS12J-2 is also able to generate 1-2 bp indels and/or single nucleotide changes at lower frequencies. However, the current experimental setup and data analysis method are not able to determine if such variations observed are caused by CAS12J-2 editing or caused by experimental imperfections which cannot be avoided (e.g. PCR inaccuracy, sequencing errors).

TABLE 1-2
Amplicon sequencing results from protoplasts transfected with
pCAMBIA1300_pUB10_pcoCAS12J2_E9t_version2_AtPDS3_gRNA5
and incubated at 23° C. with an additional 37° C. incubation.
The column labeled “Editing Pattern” lists the mutant allele
created by in vivo CAS12J-2 editing. Editing patterns are labeled
as [position where the editing starts]:[number of
nucleotides deleted (D)]. Position 0 is between the 18th and 19th
nucleotides of the guide, such that the 18th nucleotide is position −1,
the 19th nucleotide is position +1, and so on.

| Editing Pattern | number of reads |
|---|---|
| −5:8D | 524 |
| −5:9D | 426 |
| −5:10D | 403 |
| −5:7D | 349 |
| −2:3D | 340 |
| −6:10D | 330 |
| −4:7D | 319 |
| total reads number with editing | 2691 |
| total number of reads | 2386054 |
| percent of edited reads | 0.11% |

TABLE 1-3
Amplicon sequencing results from protoplasts transfected
with RNP of CAS12J-2 protein and AtPDS3 gRNA10 and
incubated at 23° C. with an additional 37°
C. incubation. Editing patterns are labeled as in Table 1-2.

| Editing Pattern | number of reads |
|---|---|
| −5:9D | 5181 |
| −4:4D | 2147 |
| −1:5D | 1771 |
| −4:8D | 1512 |
| −2:6D | 1474 |
| −4:7D | 1316 |
| −5:8D | 1026 |
| −4:5D | 847 |
| −2:9D | 823 |
| −6:10D | 819 |
| −7:17D | 778 |
| −7:12D | 734 |
| 1:5D | 336 |
| −6:18D | 329 |
| −8:11D | 299 |
| −5:19D | 289 |
| −5:14D | 282 |
| −5:10D | 280 |
| −11:28D | 279 |
| −21:24D | 277 |
| −4:18D | 268 |
| −16:24D | 265 |
| −4:13D | 247 |
| −24:26D | 247 |
| −3:21D | 241 |
| −9:54D | 221 |
| −34:42D | 218 |
| −1:4D | 205 |
| 3:9D | 191 |
| −4:6D | 188 |
| −6:17D | 130 |
| −6:11D | 121 |
| −18:22D | 116 |
| total reads number with editing | 23457 |
| total number of reads | 3295708 |
| percent of edited reads | 0.71% |

TABLE 1-4
Amplicon sequencing results from protoplasts transfected
with RNP of CAS12J-2 protein and AtPDS3 gRNA8 and incubated
at 23° C. Editing patterns are labeled as in Table 1-2.

| Editing Pattern | number of reads |
|---|---|
| −6:10D | 753 |
| −3:9D | 350 |
| −7:11D | 169 |
| −5:11D | 162 |
| 1:8D | 122 |
| −29:7D, −20:23D | 105 |
| total reads number with editing | 1661 |
| total number of reads | 4194455 |
| percent of edited reads | 0.04% |

Overall, the data presented in this Example demonstrates successful in vivo editing by CAS12J-2 in plant cells.

### Example 2: Detailed Characterization of CAS12J-2 Mediated Gene Editing in Plant Cells

This Example provides more detailed characterizations of CAS12J-2-mediated gene editing in plant cells described in Example 1, focused on AtPDS3 gRNA5, gRNA8 and gRNA10. Each of these three guides showed editing of the target AtPDS3 gene in Example 1. This Example demonstrates further that AtPDS3 gRNA5, gRNA8 and gRNA10 conduct editing through transfection of RNPs (CAS12J-2 protein preloaded with guide RNA) and by transfection of plasmids (containing the CAS12J-2 expression cassette and guide RNA transcription cassette). The CAS12J-2 editing in protoplast was successful both at 23° C. and also with a 37° C. incubation added in the middle of incubation at 23° C. In vitro RNP cleavage of AtPDS3 gene PCR fragment was also successful when the reaction was carried out at 23° C.

### Materials and Methods

Plasmid Cloning and RNP Reconstitution

Plasmids and RNPs are the same as those in Example 1 or were made by the methods provided in Example 1.

In Vitro RNP Cleavage Assay

The AtPDS3 gene fragment, which spans all guide RNAs, was amplified by PCR. The size of the PCR product (2.76 Kb) was checked by gel electrophoresis and extracted. The gel extracted substrate was combined with RNP in a 1:100 molar ratio (substrate/Cas12J) in 1×CB, and the reaction mixed by pipetting. The reaction was incubated at 23° C. for 2 hours, then stopped by addition of 50 μM EDTA. 1 μL of proteinase K (Invitrogen, 20 mg/μl) was added to the reaction and incubated for 20 minutes at 37° C. Then the reaction was run on a 1% agarose gel for visualization.

Protoplast Isolation and Transfection

Protoplast isolation and transfection were performed as described in Example 1, except that after RNP transfection, the total protoplast incubation time was 48 hours instead of 36 hours. For the 37° C. treatment, protoplasts were incubated first for 12 hours at 23° C., then 37° C. for 2.5 hours, then the remaining time at 23° C.

Amplicon Sequencing and Data Analysis

Amplicon sequencing and data analysis was done as described in Example 1.

### Results

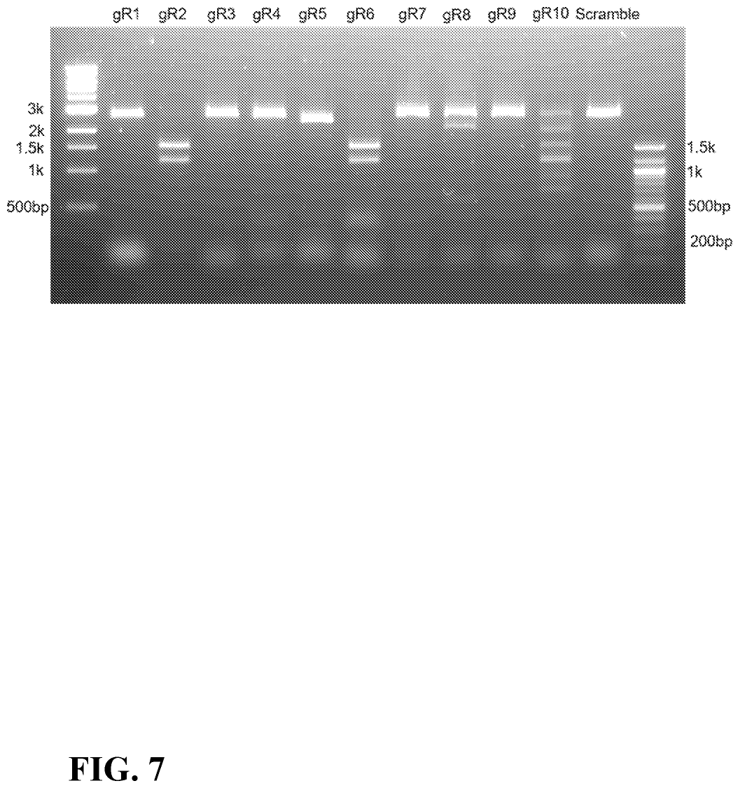

Considering that editing of the AtPDS3 gene was observed in the assays from Example 1 when protoplasts were incubated at 23° C. an in vitro RNP cleavage assay was performed to directly assess the activity of CAS12J-2 at 23° C. Cleavage of the AtPDS3 PCR fragment was observed by incubation with CAS12J-2 RNPs containing gRNA2, gRNA5, gRNA6, gRNA8 and gRNA10 at 23° C. (FIG. 7). These results directly confirm that CAS12J-2 is highly active at 23° C.

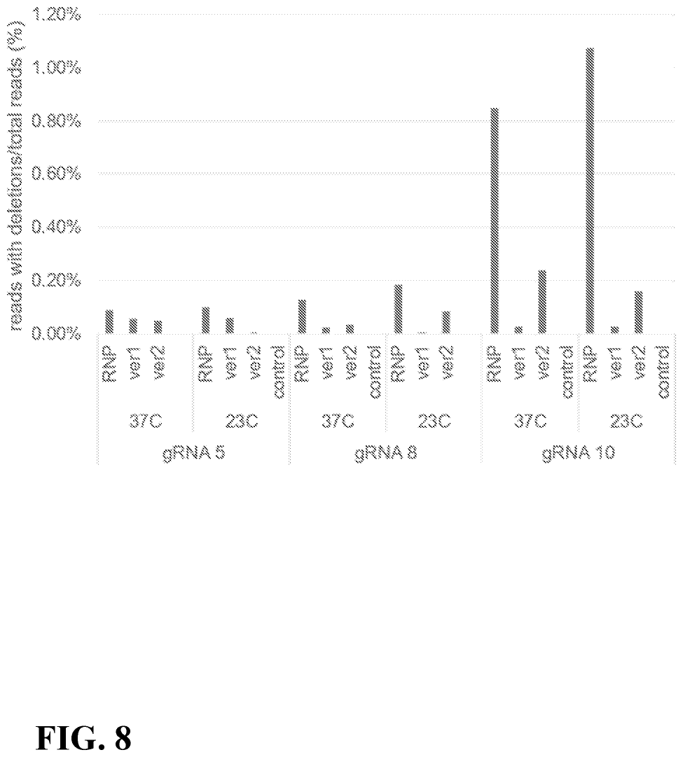

To examine CAS12J-2 editing in plant cells, Arabidopsis mesophyll protoplasts were isolated. For each guide of gRNA5, gRNA8 and gRNA10, two sets of experiments were performed: 23 C set (23° C. incubation), and 37 C set (23° C. incubation with 37° C. incubation added in the middle). For each set of experiments, version 1 and version 2 plasmids are as described in Example 1, which carry DNA cassettes encoding both the CAS12J-2 protein and guide RNA. These plasmids were transfected into protoplasts. Also, RNPs of CAS12J-2 protein and corresponding gRNAs were also transfected into protoplasts. In each set, two control samples were included where HBT-sGFP (S65T) control plasmid was transfected into protoplasts and used as control for amplicon seq. Editing of the AtPDS3 gene was observed at corresponding guide RNA target regions for all three guides, with both plasmids (ver1 and ver2) and RNPs, at both 23° C. and with the 37° C. incubation added (FIG. 8). Higher editing efficiency was observed with RNP transfection than plasmid transfection (FIG. 8).

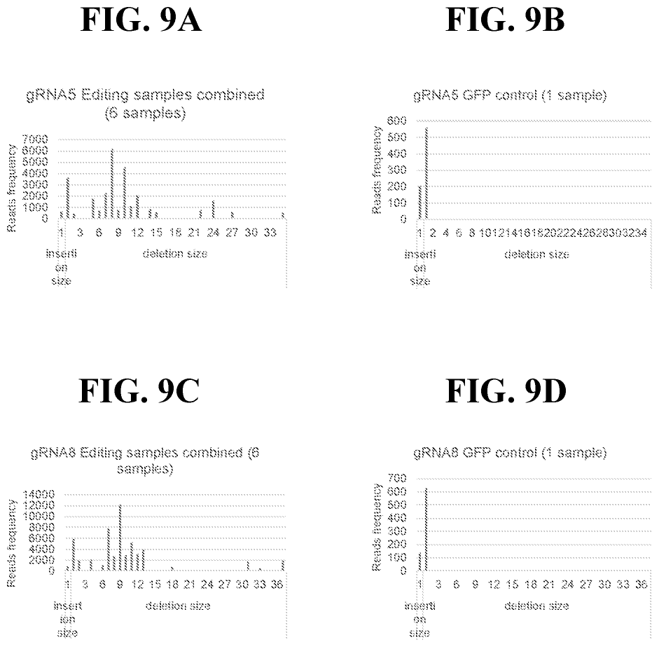

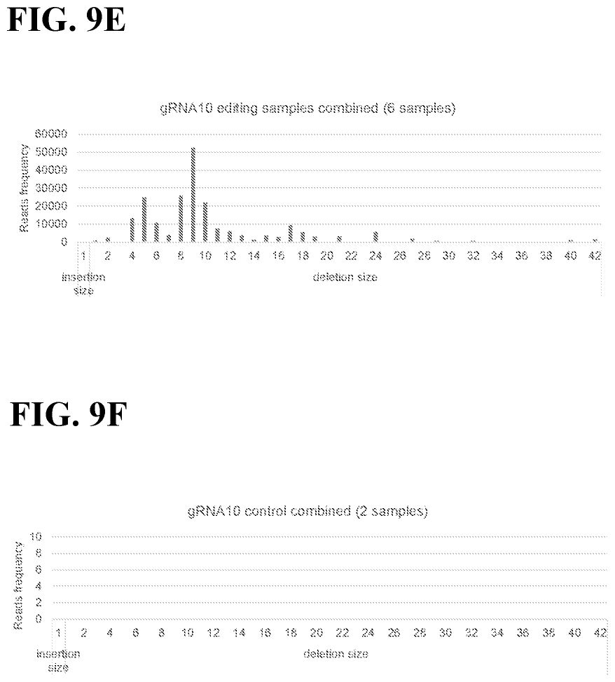

For the RNP assays, examples of editing patterns discovered in protoplast amplicons are shown in Table 2-1, Table 2-2, and Table 2-3. It was also observed that the majority of in vivo CAS12J-2 editing patterns discovered from amplicon seq are deletions, with very rare case of insertions (Table 2-1, Table 2-2, and Table 2-3). By compiling reads for each size of deletion in all editing samples for each guide, we observed that CAS12J-2 preferably creates deletions larger than 3 bp in vivo, with the most frequent alleles showing deletion of around 8-10 bp (FIG. 9A-FIG. 9F). In the case of several of the guide RNAs (e.g. gRNA8 and gRNA10), 9 bp deletions are the most frequent deletion observed (FIG. 9A-FIG. 9F), suggesting that CAS12J could be used for critical amino acid screening of proteins of interest or creating weaker alleles of genes by generating in-frame deletions.

TABLE 2-1
Protoplast amplicon sequencing results with detailed mutant alleles
created by in vivo CAS12J-2 editing with RNPs of CAS12J-2 protein
and AtPDS3 gRNA5 and incubated at 23° C. Editing patterns are shown
as: (position where the editing starts):(number of nucleotides of)
D (deletion) or I (insertion). Position 0 is between the
18^{th} and 19^{th} nucleotides of the guide, so that
the 18th nucleotide is position −1, the 19th
nucleotide is position +1 and so on.

| Editing Pattern | number of reads |
|---|---|
| −7:12D | 2069 |
| −5:24D | 1607 |
| [symbol] :7D | 1334 |
| −5:8D | 1247 |
| −6:10D | 684 |
| total reads number with editing | 6941 |
| total number of reads | 6903155 |
| percent of edited reads | 0.10% |

[symbol]

indicates data missing or illegible when filed

TABLE 2-2
Protoplast amplicon sequencing results with detailed
mutant alleles created by in vivo CAS12J-2 editing
with RNPs of CAS12J-2 protein and AtPDS3 gRNA8 and
incubated at 23° C. Labels are as in Table 2-1.

| Editing Pattern | number of reads |
|---|---|
| −3:9D | 5080 |
| −3:7D | 2762 |
| −6:12D | 2307 |
| −5:31D | 1762 |
| −5:11D | 1544 |
| −3:13D | 1423 |
| 1:4D | 1392 |
| −7:11D | 1285 |
| −5:8D | 721 |
| total reads number with editing | 18276 |
| total number of reads | 9843693 |
| percent of edited reads | 0.19% |

TABLE 2-3
Protoplast amplicon sequencing results with detailed
mutant alleles created by in vivo CAS12J-2 editing
with RNPs of CAS12J-2 protein and AtPDS3 gRNA10 and
incubated at 23° C. Labels are as in Table 2-1.

| Editing Pattern | number of reads |
|---|---|
| −5:9D | 19639 |
| −5:8D | 7205 |
| −1:5D | 7026 |
| − [symbol] :10D | 6231 |
| −4:8D | 5287 |
| −4:5D | 4782 |
| −2:6D | 3132 |
| −2: [symbol] D | 2878 |
| −7:12D | 2839 |
| −1:4D | 2826 |
| −9:11D | 2494 |
| −7:17D | 2426 |
| −5:10D | 2348 |
| − [symbol] :19D | 1840 |
| −4:7D | 1697 |
| −4:4D | 1594 |
| −5:18D | 1534 |
| −6:42D | 1533 |
| −4:17D | 1354 |
| −14:19D | 1252 |
| −8:16D | 1194 |
| −4:12D | 1192 |
| −3:21D | 1143 |
| −6:17D | 1085 |
| −4:10D | 1064 |
| −6:5D | 1025 |
| −2:2D, 3:5I | 955 |
| −8:32D | 716 |
| −6:11D | 649 |
| −5:14D | 636 |
| −5:15D | 632 |
| −7:11D | 6 [symbol] 7 |
| −3:8D | 589 |
| −4:13D | 546 |
| total reads number with editing | 91950 |
| total number of reads | 8572470 |
| percent of edited reads | 1.07% |

[symbol]

indicates data missing or illegible when filed

### Summary and Applications

CAS12J, a newly discovered subtype of Cas proteins which exclusively resides in Phage genomes, is the smallest Cas protein sub-type that are shown to be functional for cutting double stranded DNA. The CAS12J protein sizes range from around 50 KD to 90 KD, which are much smaller than that of Cas9 (162 KD) and Cas12a (also called cpf1, 151 KD). This exceptionally small size of CAS12J may allow for use of this protein in various CRISPR-based nucleic acid editing applications, such as packaging them into plant virus vectors which have cargo size limitations.

Due to the original host environment where Cas9 and Cas12a proteins evolved, these proteins require a relatively high temperature to exert optimal activity. Cas12a usually prefers 28° C. or higher temperature, while Cas9 prefers 32° C. or higher temperature. However, the ecosystems where the CAS12J host phages are discovered are highly variable, leading to a wide optimum temperature range for CAS12J proteins. From Examples 1 and 2, CAS12J-2 was observed to be functional at both 23° C. and 37° C. without drastic difference in activity at these two temperatures. This wide optimal temperature range may allow CRISPR-Cas related tools utilizing Cas12J to be developed for plants which prefer lower temperatures, as well as for cold-blooded animals and insects.

In terms of the substrate cutting activity. Cas9 employs two nuclease domains (HNH and RuvC-like) to cleave the two strands of target DNA. The result of Cas9 cutting is a blunt end cleavage. Cas12a, on the other hand, induces 4-5 nucleotides of staggered cut with a single RuvC domain. CAS12J also uses a single RuvC domain for target cleavage, but creates longer staggers ranging from 8 to 12 nt in the CAS12J proteins tested herein. This long-staggered cut created by Cas12J may be particularly useful for various applications. For example, coupled with cellular DNA repair mechanisms. CAS12J could be used for (1) creating mutant alleles, as in the case of Cas9 and Cas12a, and (2) modulation of target DNA by supplying donor DNA. The second process could be strongly enhanced by the fact that CAS12J creates long staggered cuts. Also, as was seen in Examples 1 and 2, CAS12J-2 preferably creates longer deletions (peak frequency at 8-10nt) in vivo, allowing for a series of applications based on this, such as promoter mutation scanning.

Cas9 utilizes a crRNA:tracrRNA duplex to function as its guide RNA and needs other protein components to process pre-crRNA into mature crRNA. Although well-known single guide RNAs have been engineered for Cas9, the length of Cas9 sgRNA is significantly longer than the crRNA employed by Cas12a and CAS12J. Cas12a can process pre-crRNA into crRNA by itself with the crRNA size as 44 bp, while CAS12J also doesn't need tracrRNA and is also capable of self-processing pre-crRNA. Pre-crRNA self-processing activity could be utilized for multi-targeting by introducing a CRISPR array in the organism of interest. The size of Cas12J-2 guide RNA tested herein and shown to be functional in vivo is 25nt repeat+18nt spacer, which is on the same scale as Cas12a and much smaller than that of Cas9. Cas12J processes its gRNAs via its RuvC domain, which may help explain the compact size of Cas12J.

As was seen in Examples 1 and 2, the most common deletion event created by Cas12J-2 was 9 base pairs in length. This is in contrast to Cas9 which usually creates one basepair deletions, and Cas12A makes small deletions. Without wishing to be bound by theory, it is thought that after Cas12J-2 creates a staggered cut on a DNA molecule, the cell trims back the overhanging sequences to create the nucleotide sequence deletion. It is noteworthy that 9 is a multiple of 3, and 3 bp is the size of a codon for one amino acid. Thus, Cas12J could be used for making small in-frame deletions across a protein coding sequence for the purpose of e.g. creating weak alleles in proteins (e.g. partial loss of function). Weak alleles are often very useful in crop improvement. Examples of in-frame deletions that could be important would be in genes with several known domains, such as enzymatic domains, DNA-binding domains, etc. Cas12J could be used to make 3, 6, 9, 12, 15 or other in-frame deletions to specifically delete individual domains in a protein. An exemplary target could be the LRR domains of CLV receptor proteins.

Further, Cas12J may also find use in creating weak alleles in promoters. Cas9 and Cas12a make smaller deletions and are therefore less useful for chopping out transcription factor binding sites. The larger deletions created by Cas12J, in view of the T-rich and permissive PAM sequence used by Cas12J, may allow for a much higher range of transcription factor binding sites that can be deleted or edited with Cas12J. Promoters are usually AT-rich compared to exons, which are more GC-rich. Corn and many other plants have higher GC content in exons than introns or intergenic regions which include the promoter regions, so Cas12-based editing of AT-rich regions may find particular use in these systems to allow for finer tuning of deletions and edits.

Finally, the unique properties of Cas12J may allow this protein to be developed into a cloning reagent for use in plants. Type II restriction endonuclease systems are currently used for the cloning of guide RNAs into vectors. However, use of these systems as cloning reagents in plants is challenging given the often large size and complexity of plant vectors (e.g. plant dual vectors). In view of this, it is possible that Cas12J could be developed into an engineerable restriction enzyme similar to existing type II restriction systems used in other organisms. This may be particularly beneficial given the apparent relative ease at which Cas12J can be purified and concentrated, and its good stability. Further, the wide range of temperatures at which Cas12J is active as shown herein suggest that this protein could find use as a flexible and efficient cloning enzyme. The pattern of staggered cuts produced by Cas12J may also allow for efficient ligation.

### Example 3: Factors Influencing Transfection and Editing Efficiency

This Example outlines factors that influence the efficiency of plasmid transfection of protoplasts.

### Introduction

In regular plasmid transfection of protoplasts, the transfection efficiency is usually 60-90% with healthy protoplasts and good quality plasmid DNA (PMID: 17585298). However, the transfection efficiency can be affected by many factors such as the health of plants, plasmid DNA quality, and the plasmid: protoplast ratio. This Example explores additional factors that can influence transformation efficiency.

### Materials and Methods

Protoplast Isolation and Transfection

Protoplast isolations were performed with the same procedure as outlined in Example 1. In the “no CB buffer” sample, 10 μL of HBT-sGFP (S65T) plasmid (1 ug/ul, ABRC stock CD3-911) were added to 200 μL protoplast and briefly mixed by gently tapping tube 3-4 times. Then, 210 μL of freshly prepared PEG-CaCl₂) solution was added and mixed well by tapping the tube. After incubation at 23° C. for 10 min, 880 μL of W5 buffer was added and the tube was inverted 2-3 times to stop transfection process. Protoplasts were collected by centrifugation at 100 rcf for 2 min and resuspended gently in 1 mL WI. Then protoplasts were plated in 1 well of 6 well plates precoated with 5% calf serum. In the “with CB buffer” sample, 10 μL HBT-sGFP (S65T) plasmid (1 μg/μL) and 13 μL of 2×CB buffer (components shown in methods of Example 1) were added to 200 μL protoplasts, mixed by gentle tapping 3-4 times. Then 223 μL (to keep a 1:1 volume ratio of sample to PEG solution) of fresh PEG-CaCl₂ buffer were added and mixed well by gently tapping the tube. After incubation at 23° C. for 10 min, 880 μL of W5 buffer was added and the tube was inverted 2-3 times to stop transfection process. Protoplasts were collected by centrifugation at 100 rcf for 2 min and resuspended gently in 1 mL WI. Then protoplasts were plated in 1 well of 6 well plates precoated with 5% calf serum. Both samples were incubated at 23° C. for 10 hours.

Microscopy Assays

GFP and bright field pictures were taken with a fluorescent microscope and shared the same settings between two sets of samples. The number of cells with GFP signal and total intact cells were counted with the GFP channel picture and the brightfield picture respectively. When counting for intact cells (cells not fractured), the criteria was as follows: if the edge of a cell revealed by the picture is a round circle or a part of a round circle, the cell is counted as an intact cell.

### Results

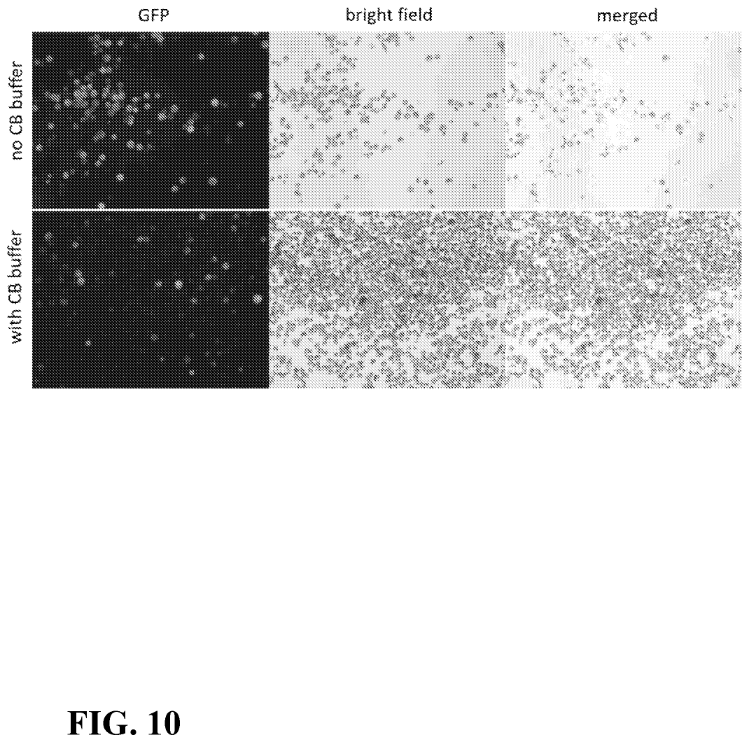

In these assays, it was discovered that adding CB buffer to the transfection reaction significantly reduces transfection efficiency as reported by GFP signal expressed from transfected HBT-sGFP (S65T) plasmid (FIG. 10 and Table 3-1). This observation suggests that in the population of protoplasts which actually received the CAS12J-2 RNPs, the editing efficiency is much higher than what was obtained by calculating transfection efficiency against the whole protoplast population.

TABLE 3-1
Summary of cell counts and transfection efficiency
from the data depicted in FIG. 10.

| | GFP positive | Total intact | Transfection |
|---|---|---|---|
| | Cell number | cell number | efficiency |
| No CB buffer | 153 | 191 | 80.1% |
| With CB buffer | 92 | 814 | 11.3% |

### Example 4: In Planta Editing with CAS12J-2 Targeting PDS3

In previous examples, it was shown that CAS12J-2 is able to conduct gene editing in plant cells by transfecting either CAS12J-2 RNP or plasmid DNA encoding CAS12J-2 and guide RNA into Arabidopsis protoplasts. In this example, transgenic plants were generated by inserting DNA encoding CAS12J-2 and guide RNA into the Arabidopsis genome using Agrobacterium transformation. Editing of the targeted gene was observed in transgenic plants grown constantly at room temperature (23° C.), as well as transgenic plants cultured initially at 28° C. for 2 weeks then transferred to room temperature. From the T2 population, transgene free seedlings that maintain the targeted gene edits were identified indicating the heritability of gene editing by CAS12J-2.

### Materials and Methods

Plasmid Cloning

Step 1: Binary vector of pCAMBIA1300_pYAO_-pcoCAS12J2_version1 MCS and pCAMBIA1300_pYAO_pcoCAS12J2_version2 MCS were constructed. These two binary vectors have the CAS12J-2 protein expression cassette with corresponding NLS and FLAG tag as described in Example 1, driven by the promoter of Yao gene. At this step, the guide RNA cassette has not been added yet. To construct these two plasmids, the following fragments were assembled in an in-fusion reaction with TAKARA in-fusion HD cloning kit: (1) pCAMBIA1300-pYAO-cas9 vector (with name as pYAO:hSpCas9 in PMID: 26524930) was digested with KpnI and BamHI, the larger fragment was gel purified, (2) Yao promoter fragment was PCR amplified from pCAMBIA1300-pYAO-cas9 vector. During PCR, >=16 bp of sequence was added by the primer which is overlapping with the pCAMBIA1300-pYAO-cas9 vector backbone fragment and with the coding sequence of CAS12J-2 protein with NLS and FLAG in version1 or version2 on the corresponding side of fragment end. (3) The coding sequences of CAS12J-2 protein with NLS and FLAG in version1 and version2 were amplified from HBT-pcoCAS12J-2 version1 and version2 described in Example 1. During PCR, >=16 bp of sequence was added by the primer which is overlapping with the pCAMBIA1300-pYAO-cas9 vector backbone fragment and the Yao promoter fragment on the corresponding side of fragment end. After the assembly of these fragments for both version1 and version2 plasmids, Sanger sequencing was used to check the sequences.

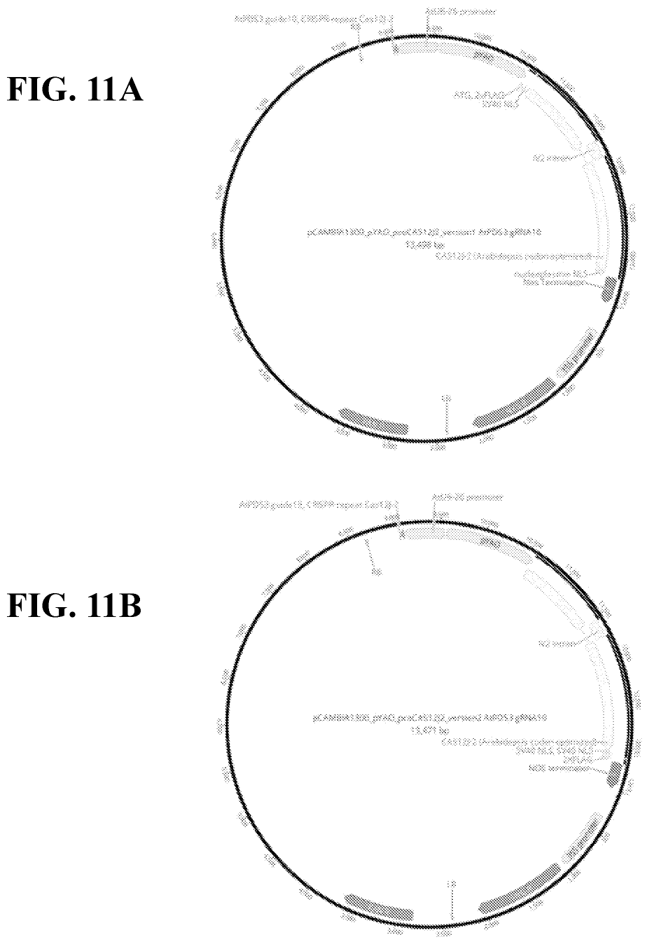

Step 2: Clone the AtU6-26 guide RNA cassette into the plasmids from step 1. This step is carried out with the same guide RNA cassette cloning method as described in Example 1 plasmid cloning method step 3. The resulting plasmid maps are shown in FIG. 11A-FIG. 11B. Maps and sequences containing the AtPDS3 gRNA10 are shown as an example. For other AtPDS3 guides, the spacer part sequence is changed according to Table 1-1.

The plasmid sequence of pCAMBIA1300_pYAO_pcoCAS12J2_version1_AtPDS3_gRNA10 is shown in SEQ ID NO: 25 and the sequence of pCAMBIA1300_pYAO_pcoCAS12J2_version2_AtPDS3_gRNA10 is shown in SEQ ID NO: 26. The corresponding plasmid sequences for other guides are only changed in the spacer sequence part according to Table 1-1. Note that the guide RNA cassette is going in reverse direction compared to the CAS12J protein encoding cassette, so the guide RNA sequence (depicted as DNA sequence) are revealed as reverse complement in the following plasmid sequences. Letters in bold indicate CAS12J-2 DNA sequence (Arabidopsis codon optimized). Letters in italic indicate the IV2 intron. Letters in bold and italic indicate guide RNA sequence (spacer part). Underlined: CAS122J repeat sequence.

Agrobacterium-Mediated Transformation

Transformation of Arabidopsis was performed with Agrobacterium strain AGL0 following the protocol described in PMID: 17406292. Arabidopsis ecotype Col-0 plants were used for transformation.

Selection of Transgenic T1 Plants

Seeds of Agrobacterium transformed plants were sterilized and plated onto ½ MS medium plates with 40 μg/ml hygromycin B (ThermoFisher 10687010). Then the seeds were stratified in dark at 4° C. for 48-72 hours. For room temperature (23° C.) selection, plates were placed into growth room at room temperature. Transgenic T1 plants were transferred from plates to soil when they can be clearly separated from plants that are not resistant to hygromycin. On hygromycin MS plates, resistant plants are able to develop normal long roots and true leaves while non-resistant plants have roots that do not elongate and do not develop true leaves. For 28° C. selection, stratified seeds on hygromycin MS plates were placed into incubator set at 28° C. Transgenic T1 plants were transferred to soil when they can be clearly separated from non-resistant plant and placed back to 28° C. incubator for a total of 2 weeks incubation at 28° C. Then the T1 plants were moved to regular growth room (room temperature).

DNA Extraction

Plant DNA was extracted with Platinum Direct PCR Universal Master Mix kit (ThermoFisher A44647500).

Sanger Sequencing and Alignment of Protein Homologs

Purified PCR products were sent to Genewiz for Sanger sequencing with proper primers. Sanger sequencing results were analyzed with Geneious software. Protein homologs alignment (for AtPDS3 homologs in different species) was performed with Clustal Omega by Geneious software.

Amplicon Sequencing

The amplicon was obtained by two rounds of PCR. Amplification primers for the first round of PCR were designed to have the 3′ sequence of the primer flanking a 200-300 bp fragment of the AtPDS3 gene around the region targeted by the guide RNA of interest. The 5′ part of the primer contains a sequence which will be bound by common sequencing primers (for reading paired-end reads, read 1 and read 2). The primers were designed so that the gRNA target sequence starts from within 100 bp of the beginning of read 1. The first round of PCR was done with Thermo Phusion enzyme and DNA extracted from the T1 generation of transgenic plants as template. After 25 cycles of amplification, the reaction was cleaned using 1× Ampure XP beads. The eluate was used as template for the second round of PCR using the Phusion enzyme and 12 cycles of amplification. The second round PCR was designed so that indexes were added to each sample. The samples were then purified using 0.8× Ampure XP. The resulting amplicons were then sent for next generation sequencing.

Amplicon Sequencing Result Analysis

Reads were first quality and adaptor trimmed with trim-galore and then mapped to AtPDS3 genomic region by BWA aligner. Sorted and indexed bam files were used as input files for further analysis by the CrispRvariants R package. Each mutation pattern with corresponding read counts were exported by the CrispRvariants R package. After assessing all control samples, a criterion to classify reads as reads with a deletion was established: only reads with a >=3 bp deletion of the same pattern (deletion of the same size starting at the same location) with >=100 reads counts from a sample are counted as reads with a deletion. This criterion is established due to the observation of 1 bp indels and occasionally, 2 bp deletions with read numbers>100 in control samples. Also observed were larger deletions that happen at very low frequencies (much lower than 100 reads) in control samples. These observations indicate that occasional PCR inaccuracy and low-quality sequencing in a small fraction of reads can result in deletion patterns with corresponding read number ranges as stated above in control samples. By employing such stringent criteria, it is believed that the deletion signals that were counted are true signal indicating editing events.

### Results

To investigate if CAS12J-2 is able to edit a target gene in transgenic plants, the Agrobacterium transformation method was used to insert DNA encoding CAS12J-2 protein and a guide RNA of interest into the Arabidopsis genome. In addition to the pCAMBIA1300 pUB10 pcoCAS12J2 E9t version1 and version2 plasmids, pCAMBIA1300 pYAO pcoCAS12J2 version1 and version2 plasmids were constructed (FIG. 11A-FIG. 11B). In these plasmids, the promoter of the YAO gene, which has high activity in dividing cells (PMID20699009), is used to drive the expression of the CAS12J-2 protein. DNA sequences encoding AtPDS3 gRNA5, gRNA8, and gRNA10 (Table 1-1) were cloned into these plasmids driven by the AtU6-26 promoter. The floral dip method (PMID: 17406292) with Agrobacterium strain AGL0 was used to transform these plasmids into wild type (Col-0 ecotype) Arabidopsis plants. T1 seedlings were selected on half MS plates with 40 μg/ml hygromycin at room temperature (23° C.) or 28° C. incubator. T1 plants which were resistant to hygromycin were transferred to soil when they could be clearly separated from non-resistant plants. After transferring to soil, T1 plants that were screened in a 28° C. incubator were placed back in the 28° C. incubator for a total of 2 weeks and then moved to room temperature. Leaves of soil grown T1 plants were collected for DNA extraction and PCR amplified for the target region (around the guide RNA sequence in the AtPDS3 gene). PCR products were analyzed by Sanger sequencing. The total numbers of T1 plants screened by Sanger sequencing for different transgenes are listed in Table 4-1.

TABLE 4-1
Summary of T1 transgenic plants screened by Sanger sequencing. The floral dip method with Agrobacterium
strain AGL0 was used to transform plasmids of interest into wild type (Col-0 ecotype) Arabidopsis
plants. T1 transgenic plants were screened by hygromycin selection at room temperature (23°
C.) or 28° C. for two weeks. Leaves of T1 plants transferred to soil were collected for DNA
extraction and PCR amplified for the target region. PCR products were analyzed by Sanger sequencing.

| | number of T1 plants | |
|---|---|---|
| | screened by sanger | |
| plasmid used to generate T1 plants | sequencing | selection condition |
| pCAMBIA1300 pUB10 pcoCAS12J2 E9t version1 AtPDS3 gR5 | 11 | 23° C. |
| pCAMBIA1300 pUB10 pcoCAS12J2 E9t version1 AtPDS3 gR8 | 37 | 23° C. |
| pCAMBIA1300 pUB10 pcoCAS12J2 E9t version1 AtPDS3 gR10 | 7 [symbol] | 23° C. |
| pCAMBIA1300 pUB10 pcoCAS12J2 E9t version1 AtPDS3 gR5 | 11 | 23° C. |
| pCAMBIA1300 pUB10 pcoCAS12J2 E9t version1 AtPDS3 gR8 | 54 | 23° C. |
| pCAMBIA1300 pUB10 pcoCAS12J2 E9t version1 AtPDS3 gR10 | 15 | 28° C. 2 weeks |
| pCAMBIA1300 pYAO pcoCAS12J2 version1 AtPDS3 gR5 | 46 | 23° C. |
| pCAMBIA1300 pYAO pcoCAS12J2 version1 AtPDS3 gR8 | 27 | 28° C. 2 weeks |
| pCAMBIA1300 pYAO pcoCAS12J2 version1 AtPDS3 gR10 | 26 | 23° C. |
| pCAMBIA1300 pYAO pcoCAS12J2 version1 AtPDS3 gR10 | 6 | 28° C. 2 weeks |
| pCAMBIA1300 pYAO pcoCAS12J2 version2 AtPDS3 gR5 | 5 | 23° C. |
| pCAMBIA1300 pYAO pcoCAS12J2 version2 AtPDS3 gR5 | 40 | 28° C. 2 weeks |
| pCAMBIA1300 pYAO pcoCAS12J2 version2 AtPDS3 gR8 | 6 | 28° C. 2 weeks |
| pCAMBIA1300 pYAO pcoCAS12J2 version2 AtPDS3 gR10 | 59 | 23° C. |

[symbol]

indicates data missing or illegible when filed

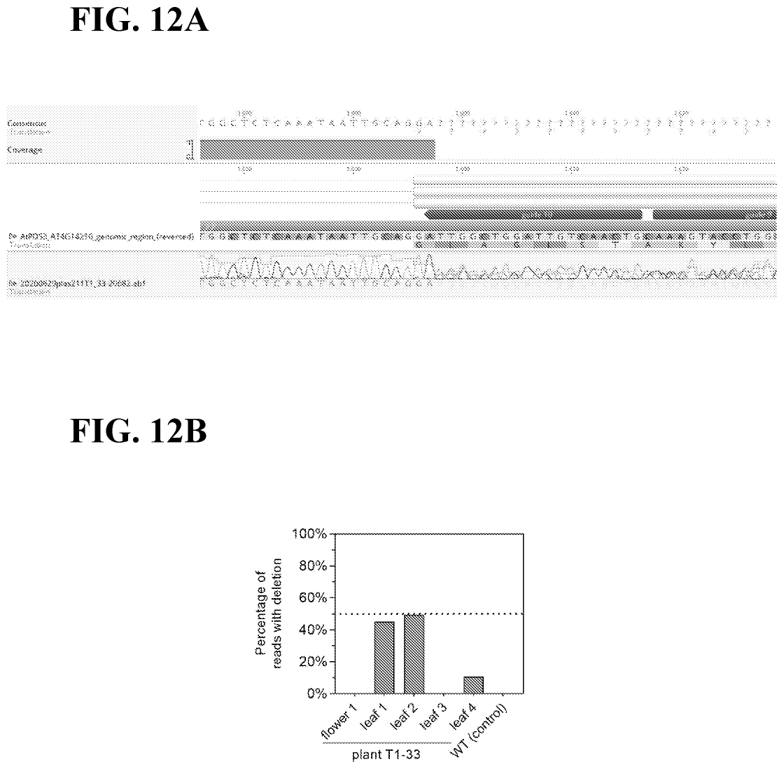

From the screen performed on the T1 plants, a T1 plant was identified that was heterozygous for a mutation in the AtPDS3 gR10 targeted region (FIG. 12A). This was T1 plant number 33 from room temperature screening of pCAMBIA1300 pUB10 pcoCAS12J2 E9t version1 AtPDS3 gR10 plasmid transformation. By performing amplicon seq with tissues from different parts of this T1 plant, we found that it was mosaic for the mutation, and thus only part of this plant carried the heterozygous mutation (FIG. 12B). The dominant mutation detected in this plant by amplicon sequencing was a 6 bp deletion in the AtPDS3 gR10 region, although small numbers of reads with other forms of deletion were also detected. The counts of different deletion patterns in leaf 2 of this plant are shown in Table 4-2.

TABLE 4-2
Detailed mutant alleles (editing pattern) detected from leaf 2
of T1 plant 33 by amplicon sequencing. Editing patterns are shown
as: (position where the editing starts):(number of nucleotides
of) D (deletion) or I (insertion). position 0 is between the
18th and 19th nucleotides of the guide, so that the 18th nucleotide
is position −1, the 19th nucleotide is position +1.

| Editing Pattern | number of reads |
|---|---|
| −5:6D | 1922589 |
| −4:6D | 2713 |
| −6:6D | 694 |
| −2:6D | 130 |
| total reads number with editing | 1926126 |
| total number of reads | 3888839 |
| percent of edited reads | 49.53% |

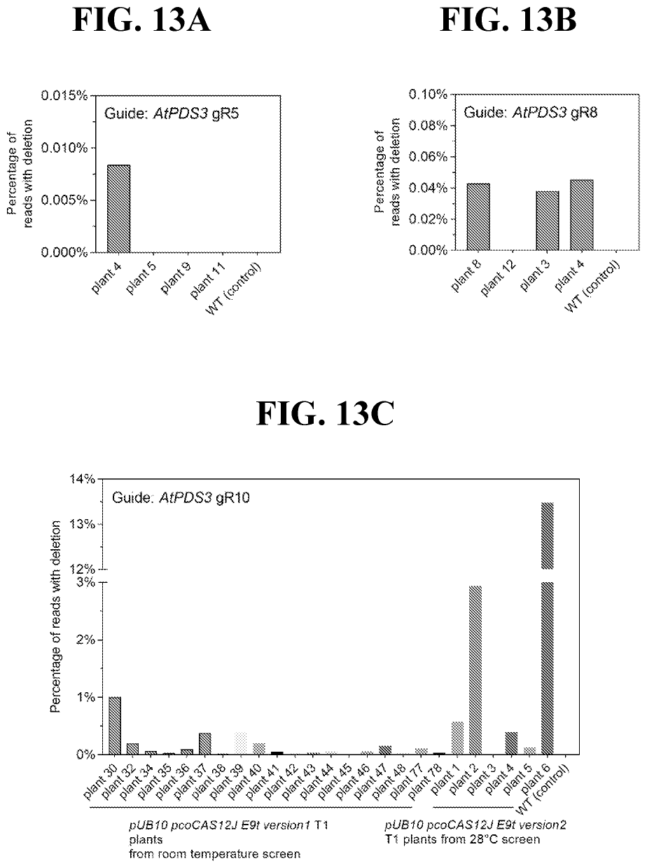

Sanger sequencing is neither powerful enough to detect mutant alleles which occur at low frequency, nor accurate at detecting mixtures of different mutant alleles. This is supported by the fact that different alleles that occur at lower frequencies, in addition to the major 6 bp deletion at gR10, were detected by amplicon sequencing in T1 plant 33 (Table 4-2). Therefore, transgenic plants with lower mutation frequencies were likely missed by the screen with Sanger sequencing, suggesting that the initial screen underestimated the rate of editing in these plants. Thus, amplicon sequencing was performed to analyze some of the transgenic T1 plants which Sanger sequencing had shown to have a wild type sequence in the target region. With this method, various forms of editing were detected which occurred at lower frequency for all three guides tested (AtPDS3 gR5, gR8 and gR10) (FIG. 13A-FIG. 13C). Editing events were detected in both version 1 and version 2 plasmids transformation (FIG. 13B and FIG. 13C. Editing was also detected in T1 plants screened both at room temperature (23° C.) and 28° C. (for gR10) (FIG. 13C). In T1 plant number 6 of pCAMBIA1300 pUB10 pcoCAS12J2 E9t version 2 AtPDS3 gR10 transformation, 13.48% of reads carried mutations of various forms, indicating that editing was occurring actively and independently in different cells of this T1 plant (Table 4-3).

TABLE 4-3
Detailed mutant allele analysis (editing patterns) detected in
T1 plant 6 containing pCAMBIA1300 pUB10 pcoCAS12J2 E9t version
2 AtPDS3 gR10 by amplicon sequencing. Editing patterns are shown
as: (position where the editing starts):(number of nucleotides
of) D (deletion) or I (insertion). position 0 is between the
18th and 19th nucleotides of the guide, so that the 18th nucleotide
is position −1, the 19th nucleotide is position +1.

| Editing Pattern | number of reads |
|---|---|
| −2:6D | 450477 |
| −12:18D | 7172 |
| −1:6D | 3849 |
| −5: [symbol] D | 3034 |
| −5:8D | 2307 |
| −4:5D | 2219 |
| −7:12D | 1675 |
| −16:24D | 14 [symbol] 9 |
| −5:6D | 1364 |
| −34:42D | 1262 |
| −4:8D | 1221 |
| −1:5D | 948 |
| −4:4D | 736 |
| −6:10D | 681 |
| −4:7D | 661 |
| −6:13D | 441 |
| −2:9D | 328 |
| −7:17D | 260 |
| −5:10D | 25 [symbol] |
| −7:11D | 210 |
| −6:11D | 200 |
| −4:6D | 157 |
| −5:14D | 147 |
| −4:8D, 9:1I | 147 |
| −5:18D | 141 |
| 2:6D | 132 |
| −6:7D | 128 |
| 1:4D | 117 |
| −8:11D | 101 |
| total reads number with editing | 481 [symbol] 13 |
| total number of reads | 3573664 |
| percent of edited reads | 13.48% |

[symbol]

indicates data missing or illegible when filed

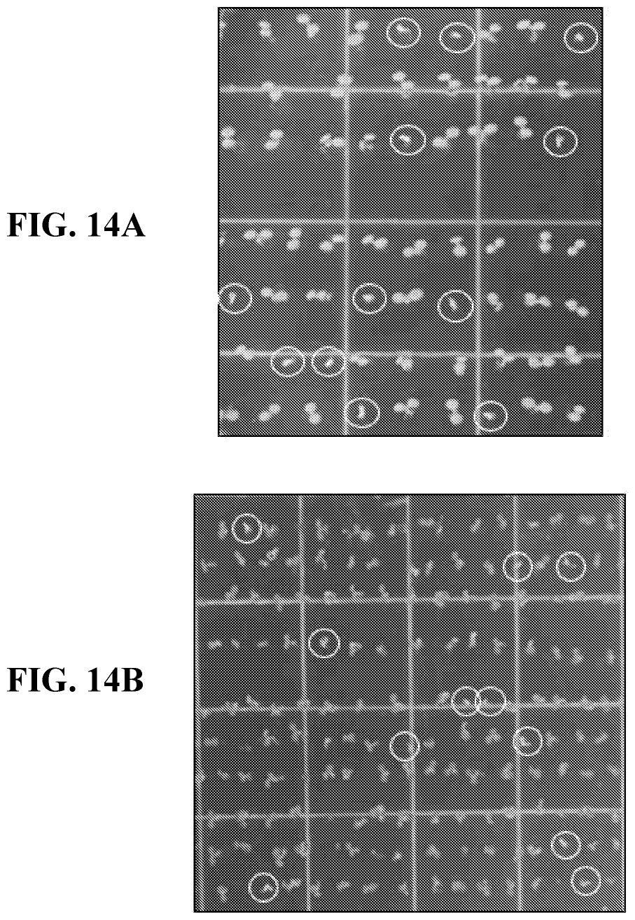

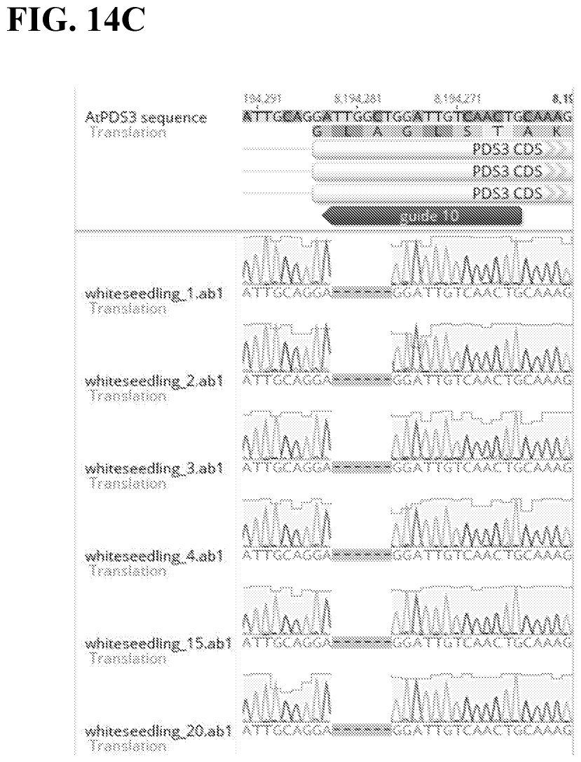

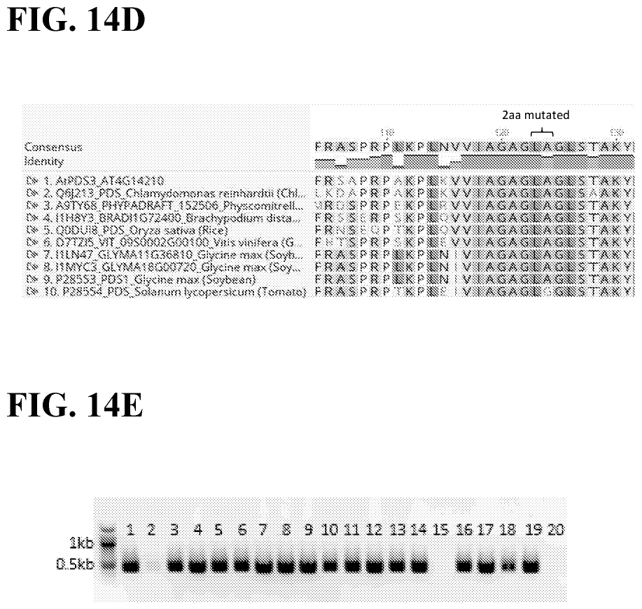

To test if the mutations generated by CAS12J-2 can be inherited in subsequent generations, seeds of pCAMBIA1300 pUB10 pcoCAS12J2 E9t version1 AtPDS3 gR10 T1 plant 33 and pCAMBIA1300 pUB10 pcoCAS12J2 E9t version 2 AtPDS3 gR10 T1 plant 6 were grown on ½ MS medium plates. The AtPDS3 gene encodes a phytoene desaturase enzyme that is essential for chloroplast development (PMID: 17486124). Disruption of this gene function results in albino and dwarfed seedlings (PMID: 17486124). It was observed that in the earlier batch of seeds harvested from T1 plant 33 (produced by the first set of flowers), a significant number of seedlings appeared as albino and dwarf (12 out of 60 in the image in FIG. 14A). In the later batch of harvested seeds of this T1 plant (those produced by flowers that developed later in development), there were also some albino/dwarf seedlings, but a lower number relative to the normal seedlings (11 out of 149 in the image in FIG. 14B). 20 albino/dwarf seedlings were collected individually for DNA extraction and the AtPDS3 gR10 target region was PCR amplified for Sanger sequencing. All 20 seedlings were homozygous for a 6 bp deletion at the gR10 target region (FIG. 14C), which was the major mutation allele observed by amplicon sequencing in the T1 plant 33 leaf tissue (Table 4-2). This 6 bp deletion is located in the coding sequence of AtPDS3 gene, resulting in the loss of two amino acids. The fact that deletion of these two amino acids caused an atpds3 mutant phenotype indicates that these two amino acids are important for the function of the AtPDS3 protein. Consistent with this finding, it was found, by aligning the protein sequences of different AtPDS3 homologs from different species, that these two amino acids were highly conserved across species (FIG. 14D), indicating an important role of these amino acids over evolutionary time. PCR amplification for the CAS12J-2 transgene was also performed to test if the 20 albino/dwarf T2 seedlings carried the transgene (FIG. 14E). As expected from genetic segregation, some of the T2 seedlings no longer contained the CAS12J-2 transgene (seedling 15 and 20). This result shows that the 6 bp atpds3 mutation was created in the T1 plants and inherited into the T2 plants in the absence of the CAS12J-2 transgene (which would have been hemizygous in the T1 plants) confirming the germline transmission (heritability) of the CAS12J-2 generated mutation in AtPDS3. This experiment represents an example of utilizing CAS12J-2 to generate in-frame deletions.

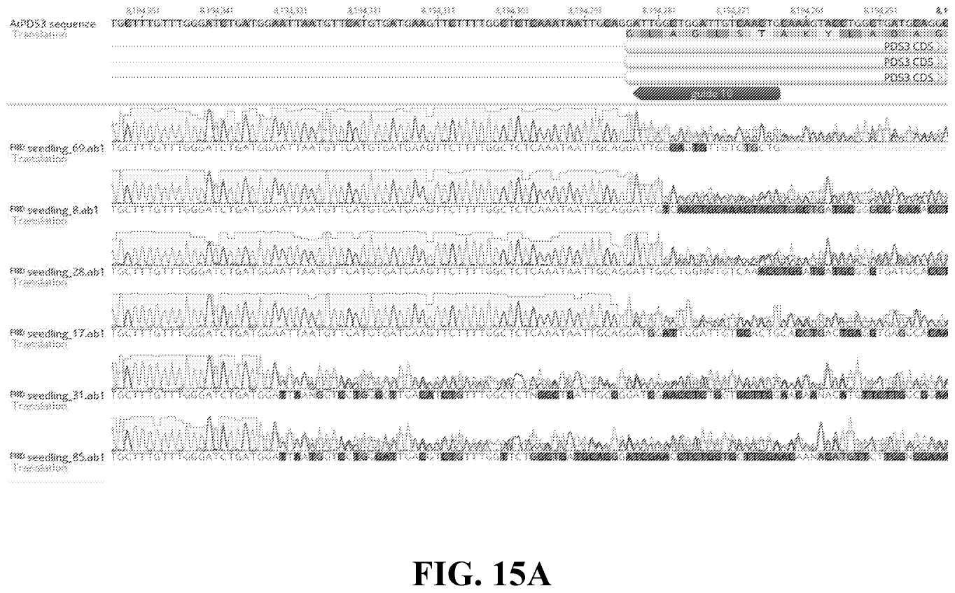

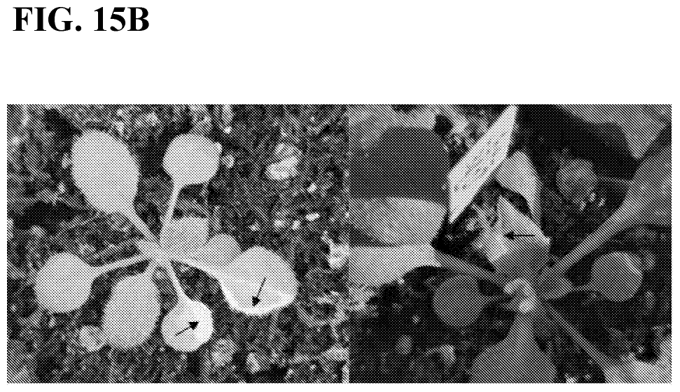

The pCAMBIA1300 pUB10 pcoCAS12J2 E9t version 2 AtPDS3 gR10 T1 plant 6 offspring population (96 T2 seedlings screened) was also analyzed, and 6 seedlings were identified that were heterozygous for mutation of the AtPDS3 gR10 target region (FIG. 15A). In addition, in one of these 6 T2 plants that were CAS12J-2 transgene positive and heterozygous for mutation at the AtPDS3 gR10 target region, albino sectors were also observed. This indicates that CAS12J-2 is actively editing the remaining wild type AtPDS3 allele in this T2 plant, leading to segments of this plant that are missing functional AtPDS3 protein (FIG. 15B, right). White sectors were also observed on a T2 plant from pCAMBIA1300 pUB10 pcoCAS12J2 E9t version1 AtPDS3 gR10 T1 plant 33 that was heterozygous for mutation of AtPDS3, again suggesting active editing of the remaining wild type allele in these plants during somatic development (FIG. 15B, left).

### Example 5: Editing with CAS12J-2 Targeting FWA in Protoplasts

In previous examples. AtPDS3 was used as a target gene for CAS12J-2 mediated editing. However, CAS12J-2 mediated editing would be useful for editing any plant gene. In this example, RNPs consisting of CAS12J-2 protein loaded with CAS12J-2 guide RNAs for the promoter region of the Arabidopsis FWA gene were introduced into protoplasts prepared from wild type plants or fwa epi-mutant plants. The data shows that CAS12J-2 is able to conduct gene editing in the promoter region of FWA gene under both repressive and active chromatin states, with editing efficiency much higher under active chromatin state compared to that under repressive chromatin state.

### Materials and Methods

RNP Reconstitution

Guide RNAs were synthesized (25nt repeat+20nt spacer as shown in Table 5-1) by Synthego. 5 nmol dry RNA was dissolved by adding 10 ul DEPC-treated H2O. 5 μl of the dissolved RNA was incubated at 65° C. for 3 min, then cooled down to RT. For RNP reconstitution, 3 μl of heated and cooled RNA was added to 292.2 ul 2×CB buffer, vortexed to mix and spun down. Then 4.8 μl of 250 μM CAS12J-2 protein was added and mixed by pipetting. This solution was then incubated at room temperature for 30 min. The resulting solution contains 4 μM of RNP in 2×CB buffer. 2×CB: 20 mM Hepes-Na, 300 mM KCl. 10 mM MgCl₂, 20% glycerol, 1 mM TCEP, PH 7.5. Special care was taken to keep all reagents RNase free.

TABLE 5-1
Guide RNA sequences used for RNP
reconstitution targeting the FWA
gene promoter region. Guide RNAs
are composed of two parts: repeat
and spacer, with spacer at the 3′
side of the repeat. A common 25 nt
repeat with the same sequence
was used for all guide RNAs.
CAS12J-2 Guide RNA repeat sequence
(common to all guides)
CAACGAUUGCCCCUCACGAGGGGAC (SEQ ID NO: 104)

| | | | Direction |
|---|---|---|---|
| FWA | | | relative |
| Guide | Guide RNA spacer | | to |
| # | sequence | PAM | FWA gene |
| 1 | UCCCAUUCAACAUUCAUACG | TTA | forward |
| | (SEQ ID NO: 105) | | |
| 2 | UCGAAGCCCAUACAUCUUUC | TTA | forward |
| | (SEQ ID NO: 106) | | |
| 3 | UGGGCCGAAGCCCAUACAUC | TTA | forward |
| | (SEQ ID NO: 107) | | |
| 4 | UGGUUCUAUACUAAUAUCAA | TTA | forward |
| | (SEQ ID NO: 108) | | |
| 5 | AUAUUAGUAUAGAACCAUAA | TTG | reverse |
| | (SEQ ID NO: 109) | | |
| 6 | GUAUAGAACCAUAACAAAAG | TTA | reverse |
| | (SEQ ID NO: 110) | | |
| 7 | CUAAAUUUAGUAAAGAAUCA | TTA | forward |
| | (SEQIDNO: 111) | | |
| 8 | GUAAUCAAUGGUUAUUGUGA | TTA | reverse |
| | (SEQ ID NO: 112) | | |
| 9 | UGAAAUGAAAUUUAACUUUU | TTG | reverse |
| | (SEQIDNO: 113) | | |
| 10 | GUUAUCUAAAUAAAACUAGG | TTA | forward |
| | (SEQIDNO: 114) | | |
| scramble | GCGACACGACUCAUUAUAAC | NONE | |
| control | (SEQIDNO: 115) | | |

RNP In Vitro Cleavage Assay

An FWA gene fragment spanning all guide RNA target regions was amplified by PCR. The PCR product was then run on gel to check for size (1.57 Kb) and gel extracted. The gel extracted substrate was combined with RNPs (in 2×CB buffer) in a 1:100 molar ratio (substrate/Cas12J) and proper amount of RNase free water was added resulting in a final 1×CB buffer concentration, and mixed by pipetting. The reaction was incubated at 37° C. for 1 h and then stopped by adding 50 uM EDTA. 1 μl of proteinase K (Invitrogen, 20 mg/ul) was added to the reaction and incubate for 20 min at 37° C. Then the reaction was run on 2% agarose gel for visualization.

Protoplast Isolation and Transfection

Wild type (Col-0 ecotype) and fwa-4 epiallele plants were grown under a 12 h light/12 h dark photoperiod and with a relatively low light condition in an incubator. Protoplast isolation was performed strictly according to the following publication: PMID: 17585298. Special care was taken to maintain a sterile environment when preparing protoplast.

For RNP transfection, 26 μl of 4 μM RNP was first added to a round bottom 2 ml tube, followed by 200 μl of protoplasts (2×10⁵ cells/ml). Then, 2 μl of 5 μg/μl salmon sperm DNA was added and mixed gently by tapping the tube 3-4 times. Finally, 228 μl of fresh, sterile and RNase free PEG-CaCl₂ solution (PMID: 17585298) was added to the protoplast-plasmid mixture and mixed well by gently tapping the tube. The protoplasts with PEG solution were incubated at RT for 10 min, then 880 μl of W5 solution (PMID: 17585298) was added and mixed with the protoplasts by inverting the tube 2-3 times to stop the transfection. Protoplasts were harvested by centrifuging tubes at 100 rcf for 2 min and resuspended in 1 ml of WI solution. They were then plated in 6-well plates pre-coated with 5% calf serum. These 6-well plates were then incubated either at room temperature for 48 h (23° C. set) or at 23° C. for 12 hours and then at 37° C. for 2.5 hours, and finally, moved back to 23° C. for 33.5 hours (37° C. set). For the fwa-4 epi-allele protoplast editing. HBT-GFP plasmids were transfected and used as a negative control.

At the end of the incubations, the protoplasts were harvested by centrifugation at 100 rcf for 2-3 min. The resulting supernatant was moved to another tube and went through another centrifugation at 3000 rcf for 3 min to collect any residual protoplasts. Pellets from these two centrifugations were combined and flash frozen for further analysis.

Amplicon Sequencing

DNA was extracted from protoplast samples with Qiagen DNeasy plant mini kit. The amplicon was obtained using two rounds of PCR. Amplification primers for the first round of PCR were designed to have the 3′ sequence of the primer flanking a 200-300 bp fragment of the FWA gene around the area targeted by the guide RNA of interest. The 5′ part of the primer contains a sequence which will be bound by common sequencing primers (for reading paired-end reads, read 1 and read 2). The primers were designed so that the gRNA target sequence starts from within 100 bp of the beginning of read 1. The first round of PCR was done with the Thermo Phusion enzyme and half of all DNA extracted from a protoplast sample as template. After 25 cycles of amplification, the reaction was cleaned using 1× Ampure XP beads. The eluate was used as template for the second round of PCR using the Phusion enzyme and 12 cycles of amplification. The second round of PCR was designed so that indexes were added to each sample. The samples were then purified using 0.8× Ampure XP. Part of the purified libraries were run on a 2% agarose gel to check for size and absence of primer dimer (fragments below 200 bp considered as primer dimer). Then amplicons were sent for next generation sequencing.

Amplicon Sequencing Result Analysis

Reads were first quality and adaptor trimmed with trim-galore and then mapped to the FWA genomic region including the promoter by BWA aligner. Sorted and indexed bam files were used as input files for further analysis by the CrispRvariants R package. Each mutation pattern with corresponding read counts was exported by the CrispRvariants R package. After assessing all control samples, a criterion to classify reads as reads with a deletion was established: only reads with a >=3 bp deletion of the same pattern (deletion of same size starting at the same location) with >=100 read counts from a sample are counted as reads with a deletion. This criterion is established due to the observation of 1 bp indels and occasionally 2 bp deletions with read numbers>100 in control samples. Also observed are larger deletions that happen at very low frequencies (much lower than 100 reads) in control samples. These observations indicate that occasional PCR inaccuracy and low-quality sequencing in a small fraction of reads can result in deletion patterns with corresponding read number ranges as stated above in control samples. By employing such stringent criteria, it is believed that the deletion signals counted are true signal indicating editing events. Additionally, for FWA gR6 and gR9 targeted regions, there are long stretches of adenines a few nucleotides just after these target regions. Due to the high error rate of polymerases dealing with long stretches of adenines, reads with deletions only within these stretches of adenines were not counted as real reads with deletions.

### Results

In wild type (WT) Arabidopsis plants, the promoter of the FWA gene contains DNA methylated region and the FWA gene is silent in all adult plant tissues. FWA is only expressed by the maternal allele in the developing endosperm where it is imprinted and demethyated (PMID: 14631047). In the epiallele fwa-4, the promoter is heritably unmethylated and thus the FWA gene is expressed ectopically leading to a late flowering phenotype (PMID: 11090618). In this example, the promoter region of the FWA gene was used as another target of editing by CAS12J-2 in addition to the AtPDS3 gene. The genomic DNA sequence of the FWA gene including the promoter is as indicated in SEQ ID NO: 27. Letters in bold are coding sequence, and letters in italic are promoter region.

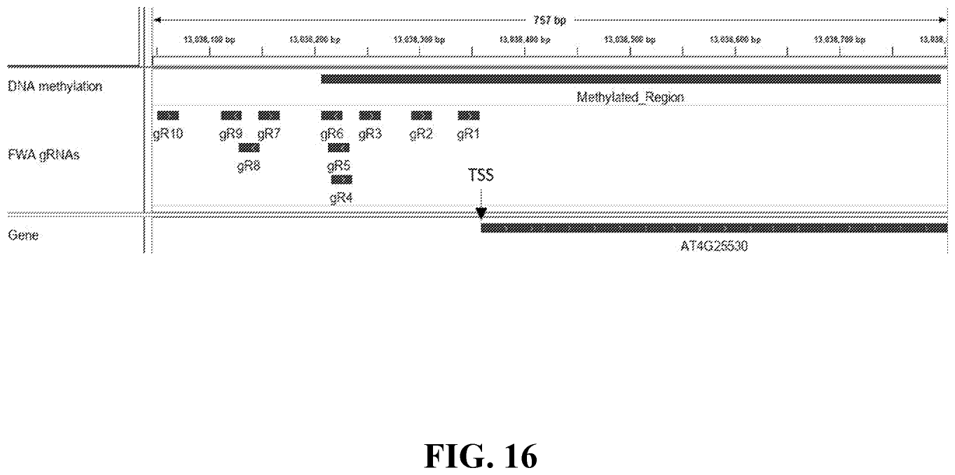

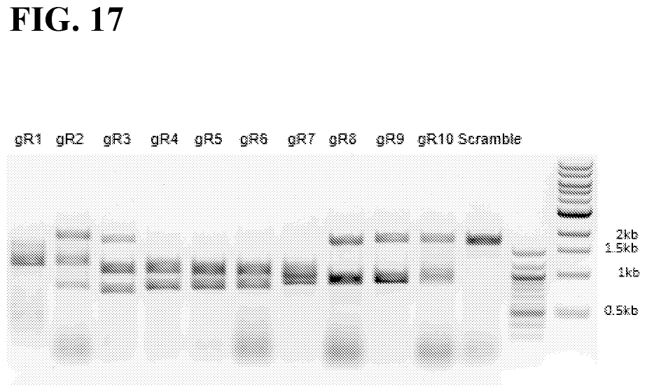

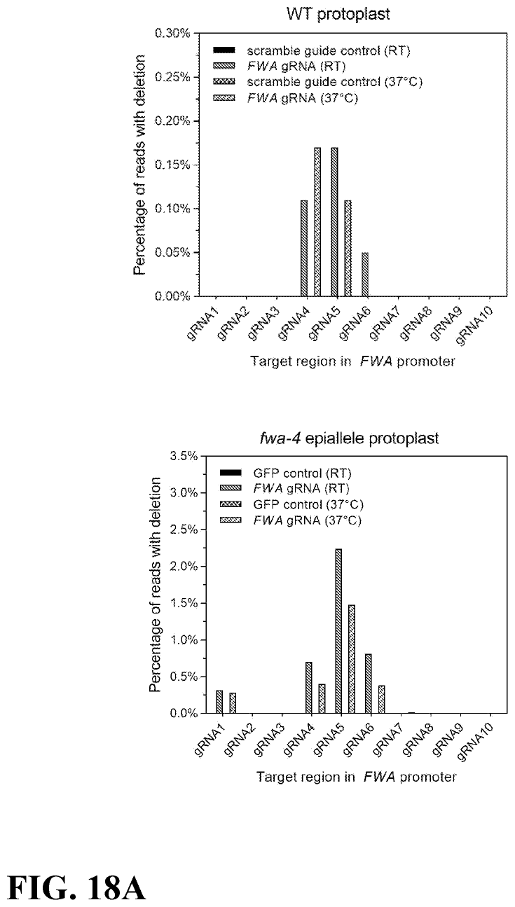

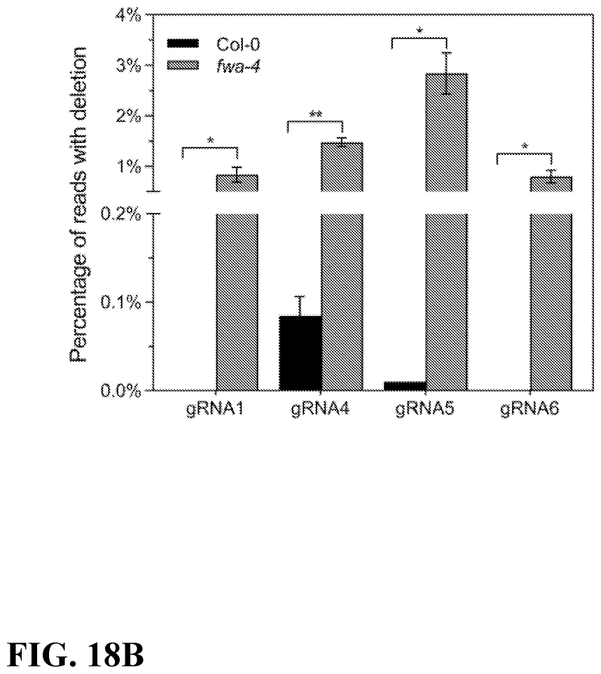

Ten guide RNAs were designed targeting the promoter region of the FWA gene, with the guide RNA sequences listed in Table 5-1 and guide RNA locations indicated in FIG. 16. In an in vitro cleavage assay with CAS12J-2 RNPs, all 10 FWA guide RNAs showed effective cleavage of the FWA gene fragment substrate, with gRNA1, gRNA4, gRNA5, gRNA6, and gRNA7 cleaving almost all of the substrate in 1 h at 37° C. (FIG. 17). CAS12J-2 RNPs were transfected into Arabidopsis mesophyll protoplasts prepared from either wild type plants (Col-0 ecotype) or fwa-4 epi-mutant plants. After the transfection, protoplasts were incubated at either room temperature (23° C.) or at room temperature with 37° C. heat step in the middle of the incubation. Successful gene editing events were observed with gRNA4, gRNA5 and gRNA6 when RNPs were transfected into wild type protoplasts, while successful gene editing events were observed with gRNA1, gRNA4, gRNA5 and gRNA6 when RNPs were transfected into fwa-4 epi-mutant protoplasts (FIG. 18). These results show that CAS12J-2 mediated gene editing is occurring when FWA is in both a repressive chromatin state (WT protoplasts where the promoter of the FWA gene contains DNA methylation and is silenced) and an active chromatin state (fwa-4 epi-mutant protoplasts where the promoter of the FWA gene is unmethylated and actively transcribed). Similar to the case when the AtPDS3 gene was used as a target of editing, deletions caused by CAS12J-2 preferably resulted in deletions of more than 3 bp, with examples of editing pattern indicated in Tables 5-2, Table 5-3, Table 5-4, Table 5-5, Table 5-6, Table 5-7, and Table 5-8.

To compare the editing efficiency under different chromatin states, an independent experiment was performed, in which WT and fwa-4 epi-mutant plants were grown under the same conditions and the protoplasts were prepared and transfected with CAS12J-2 RNPs with FWA gRNA1, gRNA4, gRNA5 and gRNA6 in parallel. Significantly higher editing efficiency was observed for each of the gRNAs used in the fwa-4 protoplasts compared to the WT protoplasts (FIG. 18B), suggesting that the CAS12J-2 mediated editing is more efficient under active chromatin state compared to repressive chromatin state. Examples of editing pattern observed in this experiment are indicated in Table 5-9, Table 5-10, Table 5-11, Table 5-12, Table 5-13 and Table 5-14. These observations suggest that lower local chromatin compaction level could potentially allow for a higher CAS12J-2 editing efficiency. Thus, it may be beneficial to choose more active and open genomic regions when designing guide RNAs.

TABLE 5-2
Detailed amplicon sequencing results of fwa epi-mutant protoplasts
transfected with CAS12J-2 RNP and FWA gRNA1. In this sample,
fwa-4 protoplasts were transfected with RNP of CAS12J-2 protein
and FWA gRNA1 and incubated at 23° C. Editing patterns are
shown as: (position where the editing starts):(number of nucleotides
of) D (deletion) or I (insertion). position 0 is between the
19th and 20th nucleotides of the guide, so that the 19th nucleotide
is position −1, the 20th nucleotide is position +1.

| Editing Pattern | number of reads |
|---|---|
| −7:12D | 1227 |
| −5:8D | 1058 |
| −6:9D | 681 |
| −5:9D | 660 |
| −3:8D | 639 |
| −9:14D | 4 [symbol] 1 |
| −2:16D | 463 |
| −6:10D | 462 |
| −6:13D | 459 |
| −7:13D | 445 |
| −5:10D | 443 |
| −9:12D | 420 |
| −1:6D | 257 |
| −4:7D | 243 |
| −8:13D | 233 |
| −6:8D | 194 |
| −7:11D | 190 |
| −6:6D | 186 |
| −8:9D | 180 |
| total reads number with deletion | 8921 |
| total number of reads | 2774111 |
| percent of reads with deletion | 0.32% |

[symbol]

indicates data missing or illegible when filed

TABLE 5-3
Detailed amplicon sequencing results of fwa epi-mutant protoplasts
transfected with CAS12J-2 RNP and FWA gRNA4. In this sample,
fwa-4 protoplasts were transfected with RNP of CAS12J-2 protein
and FWA gRNA4 and incubated at 23° C., Editing patterns
are shown as: (position where the editing starts):(number of
nucleotides of) D (deletion) or I (insertion). position 0 is
between the 19th and 20th nucleotides of the guide, so that the
19th nucleotide is position −1, the 20th nucleotide is position +1.

| Editing Pattern | number of reads |
|---|---|
| −7:7D | 4430 |
| −8:3D | 2045 |
| −7:9D | 1837 |
| −8:10D | 1666 |
| −6:9D | 1608 |
| −7:8D | 1244 |
| −7:10D | 1060 |
| −8:11D | 782 |
| −8:5D | 659 |
| −9:7D | 654 |
| −4:5D | 643 |
| −8: [symbol] D | 615 |
| −9:12D | 475 |
| − [symbol] :8D | 456 |
| −7:11D | 312 |
| −4:6D | 265 |
| −7: [symbol] D | 223 |
| −8:12D | 214 |
| − [symbol] 21D | 20 [symbol] |
| −10:13D | 199 |
| −7:2D, −1:3D [symbol] 6:4D | 184 |
| −10:11D | 179 |
| −9:14D | 173 |
| −11:17D | 121 |
| total reads number with deletion | 20247 |
| total number of reads | 2906092 |
| percent of reads with deletion | 0.70% |

[symbol]

indicates data missing or illegible when filed

TABLE 5-4
Detailed amplicon sequencing results of fwa epi-mutant protoplasts
transfected with CAS12J-2 RNP and FWA gRNA6. In this sample,
fwa-4 protoplasts were transfected with RNP of CAS12J-2 protein
and FWA gRNA6 and incubated at 23° C. Editing patterns are
shown as: (position where the editing starts):(number of nucleotides
of) D (deletion) or I (insertion). position 0 is between the
19th and 20th nucleotides of the guide, so that the 19th nucleotide
is position −1, the 20th nucleotide is position +1.

| Editing Pattern | number of reads |
|---|---|
| −4:11D | 2305 |
| 2: [symbol] D | 2126 |
| −4:5D | 1323 |
| 2:6D | 1277 |
| 2:4D | 1056 |
| −4:8D | 9 [symbol] 0 |
| −7:11D | 976 |
| −8:24D | 958 |
| −4:10D | 932 |
| −4:9D | 875 |
| −7:12D | 815 |
| −5:6D | 669 |
| −4:13D | 650 |
| −4:7D | 6 [symbol] |
| −7:10D | 602 |
| 2:7D | 505 |
| −7:17D | 4 [symbol] 5 |
| −10:16D | 46 [symbol] |
| −7:47D | 414 |
| −3:18D | 413 |
| 2:3D | 368 |
| −7: [symbol] D | 337 |
| −4:4D | 336 |
| −7:1 [symbol] D | 324 |
| −12:28D | 318 |
| −4:12D | 300 |
| −10:17D | 2 [symbol] 7 |
| −21:48D | 264 |
| −4:15D | 260 |
| −9:13D | 257 |
| −9:31D | 250 |
| −10:22D | 228 |
| −5:7D | 220 |
| −7:9D | 206 |
| −10:15D | 202 |
| −8:46D | 195 |
| −4:17D | 195 |
| −7:5D | 186 |
| −7:6D | 13 [symbol] |
| −10:14D | 13 [symbol] |
| −7:3D | 107 |
| total reads number with deletio [symbol] | 23593 |
| total number of reads | 2926828 |
| percent of reads with deletion | 0. [symbol] 1% |

[symbol]

indicates data missing or illegible when filed

TABLE 5-5
Detailed amplicon sequencing results of fwa epi-mutant protoplasts transfected
with CAS12J-2 RNP and FWA gRNA5. fwa-4 protoplasts were transfected with
RNP of CAS12J-2 protein and FWA gRNA5 and incubated at 23° C. Editing
patterns are shown as: (position where the editing starts):(number of
nucleotides of) D (deletion) or I (insertion). position 0 is between
the 19th and 20th nucleotides of the guide, so that the 19th nucleotide
is position −1, the 20th nucleotide is position +1.

| Editing Pattern | number of reads | Editing Pattern | number of reads |
|---|---|---|---|
| −1:5D | 11841 | −1:7D | 323 |
| −4:10D | 7165 | −1:13D | 302 |
| −4:9D | 4365 | −7:9D | 301 |
| 3:3D | 4133 | −11:22D | 294 |
| −1:6D | 4079 | −7:22D | 285 |
| −3:6D | 4050 | −5:7D | 2 [symbol] 5 |
| −4:11D | 330 [symbol] | −5:12D | 266 |
| −4:8D | 2771 | −25:32D | 249 |
| −7:12D | 1978 | −7:17D | 249 |
| −4:14D | 1529 | −12:32D | 238 |
| −7:11D | 1415 | −11:19D | 232 |
| 3:4D | 1284 | −2:24D | 221 |
| −4:5D | 111 [symbol] | −19:5D, −9:1 [symbol] D | 215 |
| −3:7D | 102 [symbol] | −9:19D | 205 |
| −4:15D | 987 | −14: [symbol] 1D | 20 [symbol] |
| −7:13D | 914 | −7:21D | 191 |
| 3:5D | 90 [symbol] | −7:20D | 191 |
| −9:42D | 904 | −34:36D | 190 |
| −7:6D | 870 | −3:10D | 184 |
| −4:22D | 764 | −7:10D, 14:2I | 178 |
| −9:14D | 640 | −7: [symbol] D | 159 |
| −3:12D | 601 | −28:32D | 158 |
| −5:26D | 572 | −7:23D | 152 |
| −7:10D | 469 | −9:17I | 151 |
| −4:19D | 391 | −3:2I, 3:7D | 149 |
| −9:15D | 37 [symbol] | −25:39D | 139 |
| −4:3D | 352 | −4:12D | 139 |
| −17:22D | 34 [symbol] | [symbol] :6D | 129 |
| −4:18D | 346 | −17:21D | 127 |
| −23:27D | 33 [symbol] | −12:17D | 112 |
| −4:13D | 334 | total reads number with deleti [symbol] | 66716 |
| −3:26D | 325 | total number of reads | 2979338 |

| | | |
|---|---|---|
| Continued in the table on the right | percent of reads with deletion | 2.24% |

[symbol]

indicates data missing or illegible when filed

TABLE 5-6
Detailed amplicon sequencing results of wild type (WT) protoplasts
transfected with CAS12J-2 RNP and FWA gRNA4. In this sample,
WT protoplasts were transfected with RNP of CAS12J-2 protein
and FWA gRNA4 and incubated at 23° C. Editing patterns are
shown as: (position where the editing starts):(number of nucleotides
of) D (deletion) or I (insertion). position 0 is between the
19th and 20th nucleotides of the guide, so that the 19th nucleotide
is position −1, the 20th nucleotide is position +1.

| Editing Pattern | number of reads |
|---|---|
| −7:6D | 1313 |
| −11:30D | 1202 |
| −10:13D | 1088 |
| −8:14D | 1024 |
| −8:11D | 399 |
| total reads number with deletion | 5026 |
| total number of reads | 4585427 |
| percent of reads with deletion | 0.11% |

TABLE 5-7
Detailed amplicon sequencing results of wild type (WT) protoplasts
transfected with CAS12J-2 RNP and FWA gRNA5. In this sample,
WT protoplasts were transfected with RNP of CAS12J-2 protein
and FWA gRNA5 and incubated at 23° C. Editing patterns are
shown as: (position where the editing starts):(number of nucleotides
of) D (deletion) or I (insertion). position 0 is between the
19th and 20th nucleotides of the guide, so that the 19th nucleotide
is position −1, the 20th nucleotide is position +1.

| Editing Pattern | number of reads |
|---|---|
| −1:5D | 3120 |
| −1:6D | 1522 |
| −4:14D | 1261 |
| −4:10D | 1085 |
| −4:11D | 721 |
| −9:14D | 618 |
| −7:11D | 553 |
| −4:15D | 300 |
| −4:13D | 119 |
| −3:12D | 104 |
| total reads number with deletion | 9403 |
| total number of reads | 5629525 |
| percent of reads with deletion | 0.17% |

TABLE 5-8
Detailed amplicon sequencing results of wild type (WT) protoplasts
transfected with CAS12J-2 RNP and FWA gRNA6. In this sample,
WT protoplasts were transfected with RNP of CAS12J-2 protein
and FWA gRNA6 and incubated at 23° C. Editing patterns are
shown as: (position where the editing starts):(number of nucleotides
of) D (deletion) or I (insertion). position 0 is between the
19th and 20th nucleotides of the guide, so that the 19th nucleotide
is position −1, the 20th nucleotide is position +1.

| Editing Pattern | number of reads |
|---|---|
| −4:10D | 1289 |
| −7:11D | 1022 |
| −4:11D | 803 |
| −7:12D | 155 |
| total reads number with deletion | 3269 |
| total number of reads | 6130572 |
| percent of reads with deletion | 0.05% |

TABLE 5-9
Detailed amplicon sequencing results of WT protoplasts transfected with CAS12J-2 RNP and
FWA gRNA4. In this sample, WT protoplasts were transfected with RNP of CAS12J-2 protein
and FWA gRNA4 and incubated at 23° C. Two transfections were performed: replicate
1 is shown on the left and replicate 2 is shown on the right. Editing patterns are shown
as: (position where the editing starts):(number of nucleotides of) D (deletion) or I
(insertion). position 0 is between the 19th and 20th nucleotides of the guide, so that
the 19th nucleotide is position −1, the 20th nucleotide is position +1.

| Editing Pattern | number of reads | Editing Pattern | number of reads |
|---|---|---|---|
| − [symbol] : [symbol] D | 28 [symbol] | −7:7D | 355 |
| −8:1 [symbol] D | 1 [symbol] 7 | − [symbol] : [symbol] D | 299 |
| −7:9D | 19 [symbol] | −7:10D | 273 |
| −7: [symbol] D | 181 | −7: [symbol] D | 166 |
| −7: [symbol] D | 1 [symbol] | − [symbol] :7D | 148 |
| − [symbol] : [symbol] D | 1 [symbol] | −8:10D | 127 |
| −7:11D | 1 [symbol] 7 | −9:7D | 10 [symbol] |
| total reads number with deletion | 1273 | −7: [symbol] D | 10 [symbol] |
| total number of reads | 172 [symbol] 68 | − [symbol] :11D | 101 |
| percent of reads with deletion | 0.07% | total reads number with deletion | 16 [symbol] 3 |
| | | total number of reads | 1692102 |
| | | percent of reads with deletion | 0.10% |

[symbol]

indicates data missing or illegible when filed

TABLE 5-10
Detailed amplicon sequencing results of WT protoplasts transfected with CAS12J-2 RNP and
FWA gRNA5. In this sample, WT protoplasts were transfected with RNP of CAS12J-2 protein
and FWA gRNA5 and incubated at 23° C. Two transfections were performed: replicate
1 is shown on the left and replicate 2 is shown on the right. Editing patterns are shown
as: (position where the editing starts):(number of nucleotides of) D (deletion) or I
(insertion). position 0 is between the 19th and 20th nucleotides of the guide, so that
the 19th nucleotide is position −1, the 20th nucleotide is position +1.

| Editing Pattern | number of reads | Editing Pattern | number of reads |
|---|---|---|---|
| −1:5D | 127 | −4:10D | 131 |
| total reads number with deletion | 127 | total reads number with deletion | 131 |
| total number of r [symbol] ds | 1529113 | total number of reads | 1 [symbol] |
| percent of reads with deletion | 0.01% | percent of reads with deletion | 0.01% |

[symbol]

indicates data missing or illegible when filed

TABLE 5-11
Detailed amplicon sequencing results of fwa-4 epi-mutant protoplasts transfected with CAS12J-2
RNP and FWA gRNA1. In this sample, fwa-4 protoplasts were transfected with RNP of CAS12J-2
protein and FWA gRNA1 and incubated at 23° C. Two transfections were performed: replicate
1 is shown on the left and replicate 2 is shown on the right. Editing patterns are shown
as: (position where the editing starts):(number of nucleotides of) D (deletion) or I (insertion).
position 0 is between the 19th and 20th nucleotides of the guide, so that the 19th nucleotide
is position −1, the 20th nucleotide is position +1.

| Editing Pattern | number of reads | Editing Pattern | number of reads |
|---|---|---|---|
| − [symbol] : [symbol] D | 2 [symbol] 0 | − [symbol] :10D | 14 [symbol] 0 |
| − [symbol] : [symbol] D | 22 [symbol] 8 | − [symbol] :8D | 122 [symbol] |
| −7:11D | 1637 | − [symbol] :8D | [symbol] 73 |
| −3: [symbol] D | 11 [symbol] 0 | −6:9D | 774 |
| − [symbol] :12D | 730 | − [symbol] :11D | 744 |
| − [symbol] :10D | 62 [symbol] | − [symbol] :10D | 591 |
| −6:8D | 596 | − [symbol] :10D | 464 |
| −1:4D | [symbol] | −7:12D | 434 |
| −12:1 [symbol] D | 530 | − [symbol] :4D | 42 [symbol] |
| − [symbol] D | 442 | −6:6D | 412 |
| −8:10D | [symbol] | − [symbol] :13D | 401 |
| − [symbol] :8D | [symbol] | − [symbol] :12D | 371 |
| −7:1 [symbol] D | 380 | − [symbol] D | 368 |
| −5:4D | [symbol] 77 | − [symbol] :4D | 355 |
| − [symbol] :14D | [symbol] 71 | − [symbol] :8D | 32 [symbol] |
| − [symbol] : [symbol] D | 344 | −1 [symbol] :1 [symbol] D | [symbol] 01 |
| −7:12D | 325 | − [symbol] :14D | 2 [symbol] 4 |
| −7:10D | 2 [symbol] | − [symbol] :1 [symbol] D | 2 [symbol] 1 |
| −10: [symbol] D | 2 [symbol] 4 | − [symbol] D | 273 |
| −7:23D | 240 | −12:16D | 272 |
| −4:5D | 2 [symbol] 0 | − [symbol] :11D | 2 [symbol] 2 |
| −1 [symbol] :23D | 216 | −4: [symbol] , [symbol] | 251 |
| −1:10D | 21 [symbol] | −4:7D | 24 [symbol] |
| − [symbol] :1 [symbol] D | 20 [symbol] | −10:22D | 244 |
| − [symbol] :11D | 20 [symbol] | − [symbol] : [symbol] | 243 |
| −20: [symbol] | [symbol] 8 | − [symbol] :21D | 2 [symbol] |
| −13:1 [symbol] D | 197 | − [symbol] : [symbol] D | 227 |
| −1: [symbol] D | 19 [symbol] | −3:6D | 20 [symbol] |
| −12:15D | 1 [symbol] 2 | −12:15D | 208 |
| −4:2 [symbol] D | 1 [symbol] 0 | −4: [symbol] D | 20 [symbol] |
| − [symbol] :13D | 170 | −2: [symbol] D | 137 |
| −12:11D | 1 [symbol] | −1: [symbol] D | 117 |
| − [symbol] D | 16 [symbol] | −9:4D | 10 [symbol] |
| −11:12D | 165 | total reads number with deletion | 1 [symbol] 444 |
| −5:5D | 156 | total number of r [symbol] ds | 1851732 |
| −10:11D | 14 [symbol] | percent of read [symbol] wit [symbol] deletion | 0.73% |
| −5:11 [symbol] − [symbol] | [symbol] 23 | | |
| −5: [symbol] D | [symbol] | | |
| −7:19D | 111 | | |
| −4:16D | 107 | | |
| −11:1 [symbol] D | 107 | | |
| total reads numbers with deletio [symbol] | 1802 [symbol] | | |
| total number of reads | 191764 [symbol] | | |
| percent of reads wit [symbol] deletion | 0. [symbol] 4% | | |

[symbol]

indicates data missing or illegible when filed

TABLE 5-12
Detailed amplicon sequencing results of fwa-4 epi-mutant protoplasts transfected with CAS12J-2
RNP and FWA gRNA4. In this sample, fwa-4 protoplasts were transfected with RNP of CAS12J-2
protein and FWA gRNA4 and incubated at 23° C. Two transfections were performed, replicate
1 is shown on the left and replicate 2 is shown on the right. Editing patterns are shown
as: (position where the editing starts):(number of nucleotides of) D (deletion) or I (insertion).
position 0 is between the 19th and 20th nucleotides of the guide, so that the 19th nucleotide
is position −1, the 20th nucleotide is position +1.

| Editing Pattern | number of reads | Editing Pattern | number of reads |
|---|---|---|---|
| − [symbol] : [symbol] D | [symbol] 410 | − [symbol] :7D | [symbol] 3 |
| −7: [symbol] D | 2793 | −7:6D | 22 [symbol] 4 |
| −7:8D | 16 [symbol] 4 | −7:9D | 1704 |
| −4: [symbol] D | 1512 | −9:7D | 1 [symbol] 6 |
| − [symbol] :7D | 143 [symbol] | −7: [symbol] D | 11 [symbol] |
| − [symbol] : [symbol] D | 12 [symbol] | − [symbol] : [symbol] D | 1112 |
| −7:6D | 1267 | −10:13D | 10 [symbol] 1 |
| − [symbol] :10D | 125 [symbol] | −7:12D | 923 |
| − [symbol] :14D | 112 [symbol] | −8:10D | [symbol] |
| −7:10D | 10 [symbol] | −7:10D | 5 [symbol] 3 |
| − [symbol] :11D | 958 | − [symbol] : [symbol] D | 511 |
| − [symbol] : [symbol] D | 731 | − [symbol] :16D | 4 [symbol] 2 |
| −10:1 [symbol] D | 724 | − [symbol] :11D | 4 [symbol] |
| − [symbol] : [symbol] D | 700 | − [symbol] :11D | 431 |
| − [symbol] : [symbol] D | [symbol] 8 | −3: [symbol] D | 424 |
| − [symbol] : [symbol] D | [symbol] 04 | − [symbol] : [symbol] D | 416 |
| − [symbol] :1 [symbol] D | 44 [symbol] | −8:1 [symbol] D | [symbol] 58 |
| − [symbol] : [symbol] D | 404 | − [symbol] : [symbol] D | [symbol] |
| −10:11D | 3 [symbol] 5 | −11:17D | 2 [symbol] 4 |
| − [symbol] : [symbol] D | [symbol] 2 [symbol] | − [symbol] : [symbol] D | 20 [symbol] |
| − [symbol] : [symbol] D | 310 | total reads number with del [symbol] tion | 24242 |
| − [symbol] : [symbol] D | 2 [symbol] | total number of reads | 1703365 |
| −4:7D | 191 | percent of reads w [symbol] th deletion | 1.42% |
| − [symbol] : [symbol] D | 174 | | |
| − [symbol] :12D | 174 | | |
| −4: [symbol] D | 164 | | |
| −12:11D | 1 [symbol] | | |
| −5:14D | 1 [symbol] | | |
| −10:7D | 1 [symbol] 0 | | |
| −12:14D | 13 [symbol] | | |
| −15:12D | 138 | | |
| − [symbol] :15D | 137 | | |
| −17:21D | 131 | | |
| − [symbol] :10D | 128 | | |
| −10:9D | 126 | | |
| − [symbol] :11D | 124 | | |
| −7:1 [symbol] D | 12 [symbol] | | |
| −9:1D [symbol] −2:7D [symbol] 10:4D | 109 | | |
| [symbol] : [symbol] D | 10 [symbol] | | |
| −7:1 [symbol] D | 10 [symbol] | | |
| −14:1 [symbol] D | 102 | | |
| −12:1 [symbol] D | 10 [symbol] | | |
| [symbol] reads number with deletion | 2 [symbol] 0 [symbol] | | |
| total number of reads | 1 [symbol] 7 [symbol] | | |
| percent of reads w [symbol] th d [symbol] l [symbol] ion | 1.54% | | |

[symbol]

indicates data missing or illegible when filed

TABLE 5-13
Detailed amplicon sequencing results of fwa-4 epi-mutant protoplasts transfected
with CAS12J-2 RNP and FWA gRNA5. In this sample, fwa-4 protoplasts were transfected
with RNP of CAS12J-2 protein and FWA gRNA5 and incubated at 23° C. Two
transfections were performed, replicate 1 is shown on the left and replicate
2 is shown on the right. Editing patterns are shown as: (position where the
editing starts):(number of nucleotides of) D (deletion) or I (insertion). position
0 is between the 19th and 20th nucleotides of the guide, so that the 19th nucleotide
is position −1, the 20th nucleotide is position +1.

| Editing Pattern | number of reads | Editing [symbol] | [symbol] mber of reads |
|---|---|---|---|
| − [symbol] D | [symbol] 02 [symbol] | − [symbol] D | [symbol] |
| [symbol] : [symbol] D | 4363 | [symbol] : [symbol] D | 4117 |
| −1: [symbol] D | 41 [symbol] 1 | − [symbol] D | [symbol] |
| − [symbol] : [symbol] D | [symbol] 7 [symbol] | − [symbol] : [symbol] D | [symbol] |
| −4: [symbol] D | [symbol] 987 | − [symbol] : [symbol] D | [symbol] 31 |
| − [symbol] : [symbol] 11D | [symbol] 717 | − [symbol] :11D | 23 [symbol] 8 |
| −4: [symbol] D | 3 [symbol] 60 | − [symbol] : [symbol] D | 1 [symbol] |
| − [symbol] : [symbol] D | 1 [symbol] | −4:14D | 1 [symbol] 0 [symbol] |
| − [symbol] : [symbol] D | 1104 | − [symbol] : [symbol] D | [symbol] |
| − [symbol] :8D | 1 [symbol] | − [symbol] : [symbol] D | 1 [symbol] |
| − [symbol] : [symbol] D | [symbol] 78 | −4:8D | 98 [symbol] |
| −4:14D | 9 [symbol] 7 | −7:1 [symbol] D | [symbol] |
| [symbol] | [symbol] | − [symbol] : [symbol] D | [symbol] |
| − [symbol] : [symbol] D | 7 [symbol] 4 | − [symbol] : [symbol] D | 726 |
| − [symbol] : [symbol] D | [symbol] | −7: [symbol] D | [symbol] |
| −7:1 [symbol] D | [symbol] 67 | [symbol] : [symbol] D | 6 [symbol] |
| − [symbol] :1 [symbol] D | 62 [symbol] | −4: [symbol] D | [symbol] |
| −4:1 [symbol] D | [symbol] 1 [symbol] | [symbol] : [symbol] D | 4 [symbol] |
| −7:6D | [symbol] 0 [symbol] | − [symbol] : [symbol] D | [symbol] |
| [symbol] : [symbol] D | 4 [symbol] 1 | −4:15D | 379 |
| −9:14D | 417 | −3:12D | 37 [symbol] |
| − [symbol] : [symbol] D | 382 | −4: [symbol] D | 37 [symbol] |
| − [symbol] :1 [symbol] D | [symbol] 0 [symbol] | [symbol] : [symbol] D | [symbol] 04 |
| −9:15D | 2 [symbol] | − [symbol] : [symbol] D | 2 [symbol] |
| −4: [symbol] D | 282 | −9:14D | 262 |
| − [symbol] : [symbol] D | 241 | −7:42D | 261 |
| −3: [symbol] 1D | 2 [symbol] 2 | −7: [symbol] D | 247 |
| −7: [symbol] D | 2 [symbol] 8 | −9: [symbol] D | 22 [symbol] |
| − [symbol] :18D | 214 | − [symbol] : [symbol] D | [symbol] 21 |
| −4:1 [symbol] D | [symbol] | −22:2 [symbol] D | 214 |
| − [symbol] : [symbol] D | 1 [symbol] 0 | − [symbol] : [symbol] D | 207 |
| − [symbol] :1 [symbol] D | 187 | − [symbol] : [symbol] D | 1 [symbol] 6 |
| − [symbol] : [symbol] D | 1 [symbol] | − [symbol] :18D | 1 [symbol] |
| − [symbol] :1 [symbol] D | 17 [symbol] | −12:1 [symbol] D | 17 [symbol] |
| − [symbol] : [symbol] D | 17 [symbol] | − [symbol] : [symbol] D | 17 [symbol] |
| −4: [symbol] D | [symbol] | −4: [symbol] D | 163 |
| − [symbol] :12D | [symbol] | −7:1 [symbol] D | 16 [symbol] |
| − [symbol] : [symbol] D | [symbol] | − [symbol] : [symbol] | 141 |
| − [symbol] : [symbol] D | 14 [symbol] | − [symbol] :12D | 1 [symbol] |
| −4: [symbol] D | [symbol] | − [symbol] : [symbol] D | 12 [symbol] |
| −4: [symbol] D | [symbol] 4 | − [symbol] : [symbol] D | [symbol] |
| − [symbol] :19D | 132 | − [symbol] :18D | 114 |
| − [symbol] : [symbol] D | 126 | [symbol] : [symbol] D | 10 [symbol] |
| −4:1 [symbol] D | 12 [symbol] | − [symbol] : [symbol] | 10 [symbol] |
| − [symbol] : [symbol] D | [symbol] 22 | to [symbol] reads number wi [symbol] deletion | 42 [symbol] 07 |
| − [symbol] : [symbol] D | 120 | tota [symbol] num [symbol] r of reads | 1 [symbol] 63814 |
| − [symbol] : [symbol] D | 119 | pe [symbol] ent of reads wi [symbol] de [symbol] etion | 2. [symbol] % |
| −4:4D | 108 | | |
| −12:24D | 104 | | |
| [symbol] :6D | 10 [symbol] | | |
| − [symbol] : [symbol] D | 10 [symbol] | | |
| total read [symbol] number with [symbol] l [symbol] on | 502 [symbol] | | |
| total number of reads | 16 [symbol] | | |
| p [symbol] nt of r [symbol] w [symbol] deletion | [symbol] . [symbol] % | | |

[symbol]

indicates data missing or illegible when filed

TABLE 5-14
Detailed amplicon sequencing results of fwa-4 epi-mutant protoplasts transfected with CAS12J-2
RNP and FWA gRNA6. In this sample, fwa-4 protoplasts were transfected with RNP of CAS12J-2
protein and FWA gRNA6 and incubated at 23° C. Two transfections were performed, replicate
1 is shown on the left and replicate 2 is shown on the right. Editing patterns are shown
as: (position where the editing starts):(number of nucleotides of) D (deletion) or I (insertion).
position 0 is between the 19th and 20th nucleotides of the guide, so that the 19th nucleotide
is position −1, the 20th nucleotide is position +1.

| Editing Pattern | number of reads | Editing Pattern | number of reads |
|---|---|---|---|
| 2: [symbol] D | 1914 | −4: [symbol] D | 1 [symbol] |
| −4: [symbol] D | 1455 | 2: [symbol] D | 1271 |
| −4:11D | 1374 | −4:11D | 11 [symbol] 2 |
| −4: [symbol] D | 1134 | −4: [symbol] D | 972 |
| − [symbol] : [symbol] D | [symbol] | −4:10D | [symbol] 9 |
| −4:10D | 67 [symbol] | −7:12D | 722 |
| − [symbol] D | 6 [symbol] 1 | −4: [symbol] D | [symbol] |
| 2:6D | 62 [symbol] | 2:7D | 607 |
| 2:8D | 4 [symbol] | 2: [symbol] D | 4 [symbol] |
| − [symbol] : [symbol] D | 438 | −4:1 [symbol] D | 427 |
| − [symbol] :24D | [symbol] | −10:1 [symbol] D | 419 |
| −4:12D | 3 [symbol] 4 | −8:24D | [symbol] |
| −4:17D | 27 [symbol] | −7:1 [symbol] D | 354 |
| − [symbol] : [symbol] D | 2 [symbol] | −3:18D | [symbol] 0 [symbol] |
| − [symbol] :18D | 266 | 2:4D | 273 |
| −13:24D | 260 | −4:4D | 257 |
| −1 [symbol] :17D | 260 | −4:13D | 2 [symbol] |
| −7: [symbol] D | 2 [symbol] 7 | 2: [symbol] D | 245 |
| − [symbol] :14D | 2 [symbol] 3 | −10:23D | 239 |
| 2:4D | 225 | − [symbol] :14D | 22 [symbol] |
| −7:13D | 210 | −4:12D | 211 |
| −4:16D | 207 | −10:17D | 1 [symbol] 3 |
| 2:10D | 194 | −4:7D | 177 |
| − [symbol] :1 [symbol] D | 18 [symbol] | − [symbol] : [symbol] , 6 [symbol] | 135 |
| −7:7D | 1 [symbol] | −10:18D | 124 |
| −7:16D | 179 | total reads number with deletion | 12 [symbol] 4 |
| −2:3D | 174 | total number of reads | 17711 [symbol] 6 |
| −7:8D | 163 | percent of reads with deletion | 0.71% |
| [symbol] : [symbol] D | 1 [symbol] 4 | | |
| −10: [symbol] 1D | 1 [symbol] 2 | | |
| −10:11D | 128 | | |
| −9:14D | 12 [symbol] | | |
| 2: [symbol] D | 121 | | |
| −4: [symbol] D | 121 | | |
| total reads number with deletion | 1482 [symbol] | | |
| total number of reads | 1666072 | | |
| p**ce*t of read [symbol] with deletion | 0. [symbol] % | | |

[symbol]

indicates data missing or illegible when filed

### Example 6: Editing with CAS12J-2 in Protoplasts with Guide RNAs Under Control of RNA Polymerase II Promoters

In most CRISPR/Cas systems studied to date, an RNA Polymerase III (Pol III) promoter is usually used to drive the expression of the guide RNAs. However, Pol III promoters have constitutive expression patterns meaning that the expression levels and tissue specificities are difficult to fine-tune. In this example, several RNA Polymerase II (Pol II) promoters were used to express guide RNAs for CAS12J-2, leading to successful gene editing events in protoplasts. The vast variety of Pol II promoters in plants allows for the potential of further optimization of editing efficiency by CAS12J-2 as well as precise control of the tissue or cell type being edited. The Pol II promoter-gRNA cassettes described in this example do not require special RNA processing, such as that carried out by ribozymes or the CSY4 system, because CAS12J-2 is capable of processing its own gRNAs. However, the addition of ribozyme gRNA processing machinery to the Pol II promoter-gRNA cassette was able to enhance the editing efficiency for all three promoter-gRNA cassettes tested in this Example.

### Materials and Methods

Plasmid Cloning

To build CAS12J-2 vectors with Pot II promoter driving gRNA expression, the following fragments for assembly by TAKARA in-fusion HD cloning kit (cat639650) were obtained as indicated:
(A) Common plasmid backbone with CAS12J-2 expression cassette: pCAMBIA1300 pUB10 pcoCAS12J2 E9t version2 MCS plasmid (See Example 1 for more details of this plasmid) was digested with SpeI and purified.
(B) Promoter and terminator combination fragments were obtained as follows:
   a. CmYLCV promoter and 35S terminator were PCR amplified from pMOD_B2103 plasmid (Addgene 91061).
   b. 2×35S promoter was PCR amplified from PMDC43 (ABRC stock CD3-741), HSP18.2 terminator was PCR amplified from pUBQ10_ZF108-NLS-ntDRM2cd_tHSP18.2 (Gardiner. J.; Zhao, J. M.; Chaffin, K.; Jacobsen, S. E. Promoter and Terminator Optimization for DNA Methylation Targeting in Arabidopsis. Epigenomes 2020, 4, 9).
   c. TBS insulator with UBQ10 promoter was PCR amplified as one fragment from pEG302_22aa_SunTag_nog (Addgene 120251). Rbcs-E9 terminator were amplified from pCAMBIA1300 pUB10 pcoCAS12J2 E9t version2 MCS plasmid.
   During PCR, >=16 bp of sequence was added by the primer to these fragments which are overlapping with the pCAMBIA1300 pUB10 pcoCAS12J2 E9t version2 MCS backbone fragment and with the guide RNA fragment on the corresponding side of fragment end.
(C) Different guide RNA fragments were obtained by synthesizing long DNA primers with 3′ end complementing each other within the primer pair. Then, a PCR with the primer pair without other template was used to obtain the double stranded fragment for assembly.

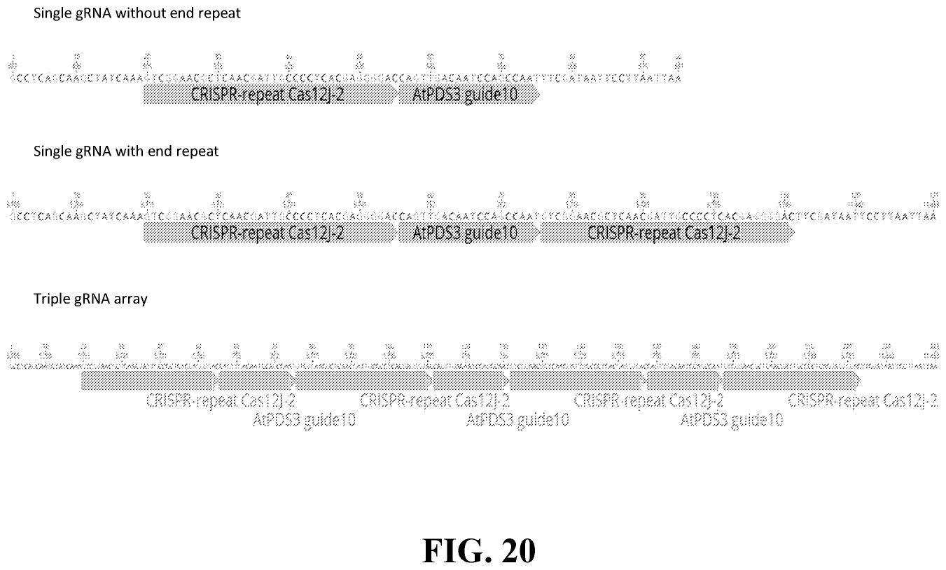

After obtaining these fragments, assembly by TAKARA in-fusion HD cloning kit (cat639650) was performed combining desired promoter-terminator combinations and guide RNA forms listed in FIG. 20. Final plasmid sequences were checked by Sanger sequencing.

The plasmid sequence of pCAMBIA1300 pUB10 pcoCAS12J2 E9t ver2 CmYLCVp AtPDS3 gRNA10 35St is set forth in SEQ ID NO: 28. This plasmid was built starting from pCAMBIA1300 pUB10 pcoCAS12J2 E9t version2, thus plasmid sequences other than the guide RNA cassette are the same as in SEQ ID NO: 14. Refer to SEQ ID NO: 14 for CAS12J coding sequence and IV2 intron sequence (note that CAS12J coding sequencing and IV2 intron sequence are revealed as reverse complement in this sequence compared to SEQ ID NO: 14). Bold letters represent the sequence of the CmYLCV promoter driving guide RNA transcription (also shown in SEQ ID NO: 29). Italic letters represent the 35s terminator sequence used in the guide RNA cassette (also shown in SEQ ID NO: 30). Bold and italic letters represent the guide RNA sequence (the spacer portion)(also shown in SEQ ID NO: 31). Underlined letters represent the CAS12J repeat sequences for the guide RNA (also shown in SEQ ID NO: 32).

The plasmid sequence of pCAMBIA1300 pUB10 pcoCAS12J2 E9t ver2 2×35Sp AtPDS3 gRNA10 HSP18t is set forth in SEQ ID NO: 33. This plasmid was built starting from pCAMBIA1300 pUB10 pcoCAS12J2 E9t version2, thus plasmid sequences other than the guide RNA cassette are the same as in SEQ ID NO: 14. Refer to SEQ ID NO: 14 for CAS12J coding sequence and IV2 intron sequence (note that CAS12J coding sequencing and IV2 intron sequence are revealed as reverse complement in this sequence compared to SEQ ID NO: 14). Bold letters represent the sequence of the 2×35S promoter driving guide RNA transcription (also shown in SEQ ID NO: 34). Italic letters represent the HSP18 terminator sequence used in the guide RNA cassette (also shown in SEQ ID NO: 35). Bold and italic letters represent the guide RNA sequence (the spacer portion)(also shown in SEQ ID NO: 36). Underlined letters represent the CAS12J repeat sequences for the guide RNA (also shown in SEQ ID NO: 37).

The plasmid sequence of pCAMBIA1300 pUB10 pcoCAS12J2 E9t ver2 insulator pUB10 AtPDS3 gRNA10 E9t is set forth in SEQ ID NO: 38. This plasmid was built starting from pCAMBIA1300 pUB10 pcoCAS12J2 E9t version2, thus plasmid sequences other than the guide RNA cassette are the same as in SEQ ID NO: 14. Refer to SEQ ID NO: 14 for CAS12J coding sequence and IV2 intron sequence (note that CAS12J coding sequencing and IV2 intron sequence are revealed as reverse complement in this sequence compared to SEQ ID NO: 14). Bold letters represent the sequence of the UBQ10 promoter driving guide RNA transcription (also shown in SEQ ID NO: 39). Italic letters represent the RbcS-E9 terminator sequence used in the guide RNA cassette (also shown in SEQ ID NO: 40). Bold and italic letters represent the guide RNA sequence (the spacer portion)(also shown in SEQ ID NO: 41). Underlined letters represent the CAS12J repeat sequences for the guide RNA (also shown in SEQ ID NO: 42). The TBS insulator sequence is shown in SEQ ID NO: 43.

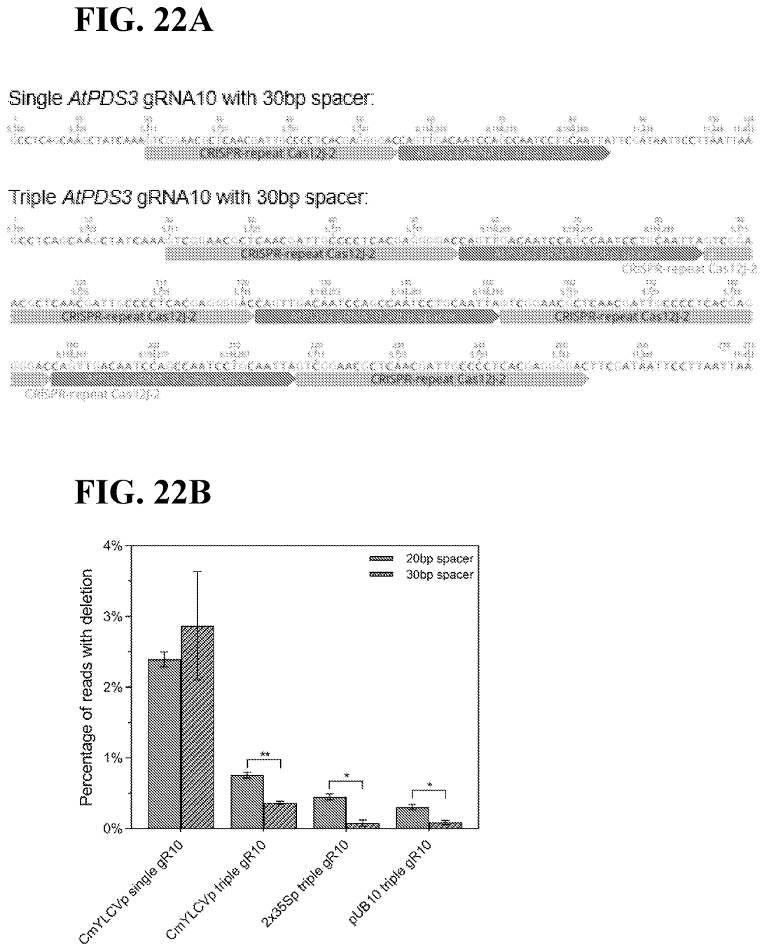

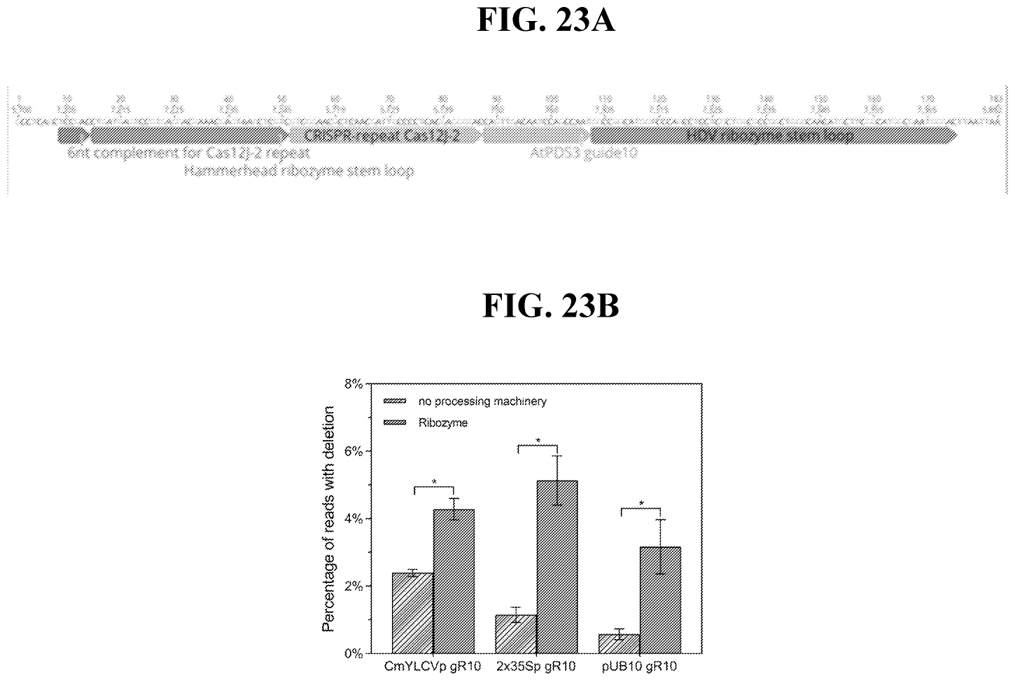

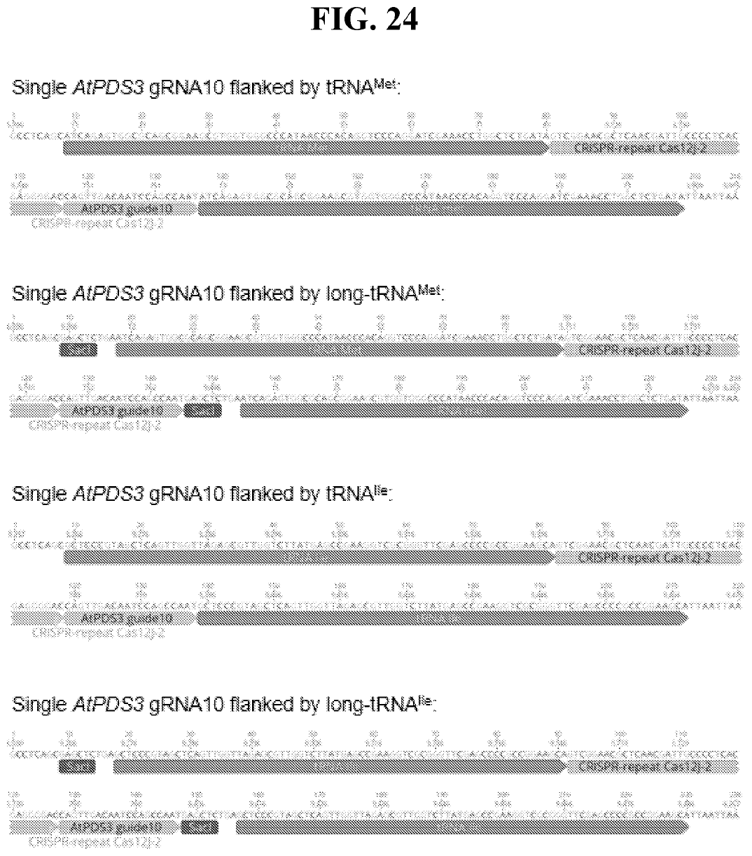

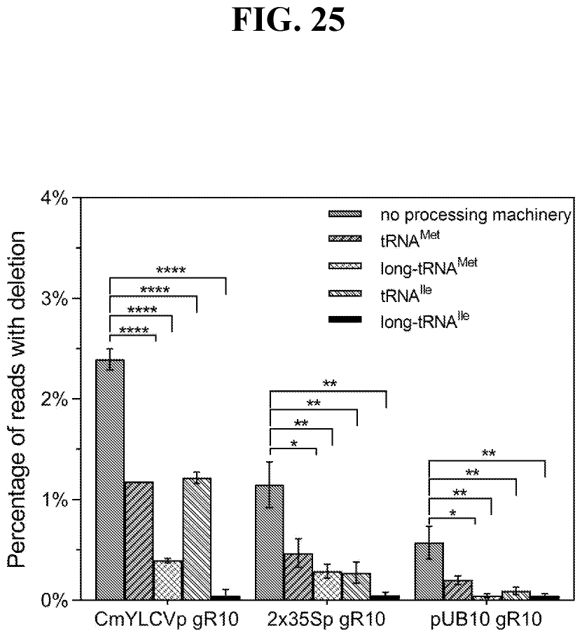

To build CAS12J-2 vectors which contain gRNA with 30 bp spacers (FIG. 22A-FIG. 22B), gRNA flanked by ribozymes (FIG. 23A-FIG. 23B) and gRNA flanked by tRNAs (as in FIG. 24 and FIG. 25) driven by Pot II promoters, the pCAMBIA1300 pUB10 pcoCAS12J2 E9t ver2 CmYLCVp AtPDS3 gRNA10 35St, pCAMBIA1300 pUB10 pcoCAS12J2 E9t ver2 2×35Sp AtPDS3 gRNA10 HSP18t and pCAMBIA1300 pUB10 pcoCAS12J2 E9t ver2 insulator pUB10 AtPDS3 gRNA10 E9t plasmids were digested with BbvCI and PacI and gel extracted for the larger fragments. These larger fragments were the vector backbone without the sequence coding the gRNA, but with the Pol II promoters and terminators for the gRNA expression. The fragments of single AtPDS3 gRNA10 with 30 bp spacer, triple AtPDS3 gRNA10 array with 30 bp spacer, ribozymes flanking single AtPDS3 gRNA10 and tRNA flanking single AtPDS3 gRNA10 were obtained by synthesizing long DNA primers with 3′ end complementing each other within the primer pair. Also, BbvCI and Pac restriction sites were included in the DNA primers on the corresponding ends. Then, PCR with the primer pairs without another template was used to obtain the double stranded fragments. The double stranded fragments were digested with BbvCI and PacI, gel extracted and ligated with the corresponding vector backbones mentioned above to generate desired constructs.

To clone the Csy4 protein coding sequence on the N-terminal of the CAS12J-2 protein coding sequence, the pCAMBIA1300 pUB10 pcoCAS12J2 E9t ver2 CmYLCVp AtPDS3 gRNA10 35St plasmid was digested with KpnI to remove the UBQ10 promoter (pUB10) and the sequence encoding the N terminal of the CAS12J-2 protein. Then, this vector backbone was mixed with the following fragments for assembly by the TAKARA in-fusion HD cloning kit (cat639650); (1) PCR amplified UBQ10 promoter (pUB10); (2) Csy4 protein coding sequence amplified from pMOD_A0801 plasmid (Addgene 91022); (3) The sequence coding for the N terminal of CAS12J-2 protein. These fragments have sequences overlapping with each other and with the vector backbone on corresponding ends added by the PCR primers. The overlapping sequence between fragment (2) and fragment (3) also contained sequences encoding an HA tag and P2A self-cleaving peptide. The resulting vector from this assembly reaction was the pCAMBIA1300 pUB10 Csy4-pcoCAS12J2 E9t ver2 CmYLCVp AtPDS3 gRNA10 35St plasmid. At this stage, Csy4 binding sites had not been added to the gRNA expression cassette yet. Then, this vector was digested with KpnI to obtain the fragment of pUB10 Csy4-pcoCAS12J2 (N-terminal). The pCAMBIA1300 pUB10 pcoCAS12J2 E9t ver2 2×35Sp AtPDS3 gRNA10 HSP18t and pCAMBIA1300 pUB10 pcoCAS12J2 E9t ver2 insulator pUB10 AtPDS3 gRNA10 E9t plasmids were also digested with KpnI and extracted for the larger fragments (vector backbone). These vector backbone fragments were ligated with the pUB10 Csy4-pcoCAS12J2 (N-terminal) fragment to obtain the pCAMBIA1300 pUB10 Csy4-pcoCAS12J2 E9t ver2 2×35Sp AtPDS3 gRNA10 HSP18t and pCAMBIA1300 pUB10 Csy4-pcoCAS12J2 E9t ver2 insulator pUB10 AtPDS3 gRNA10 E9t vectors. The detailed DNA sequence of the Csy4-CAS12J-2 expression cassette driven by UBQ10 promoter (pUB10) is indicated in SEQ ID NO: 44. Features of this expression cassette include a UBQ10 promoter (pUB10), sequence encoding Csy4 protein, sequence encoding P2A self-cleaving peptide, CAS12J coding sequence and IV2 intron sequence (same as in SEQ ID NO: 14), and E9 terminator (E9t).

To clone the Csy4 binding sites into the gRNA expression cassettes, the pCAMBIA1300 pUB10 Csy4-pcoCAS12J2 E9t ver2 CmYLCVp AtPDS3 gRNA10 35St, pCAMBIA1300 pUB10 Csy4-pcoCAS12J2 E9t ver2 2×35Sp AtPDS3 gRNA10 HSP18t and pCAMBIA1300 pUB10 Csy4-pcoCAS12J2 E9t ver2 insulator pUB10 AtPDS3 gRNA10 E9t plasmids were digested with BbvCI and PacI, and gel extracted for the larger fragments (vector backbone without the sequence coding the gRNA, but with the Pol II promoters and terminators for the gRNA expression). The fragments of single AtPDS3 gRNA10 flanked by Csy4 binding sites and triple AtPDS3 gRNA10 array with Csy4 binding sites were obtained by synthesizing long DNA primers with 3′ end complementing each other within the primer pair. Also, BbvCI and Pac restriction sites were included in the DNA primers on the corresponding ends. Then, a PCR with the primer pair without another template was used to obtain the double stranded fragments. The double stranded fragments were digested with BbvCI and PacI, gel extracted and ligated with the corresponding vector backbones to generate desired constructs.

Protoplast Isolation and Transfection

Protoplast isolation was performed strictly according to the following publication: PMID: 17585298. Special care was performed for an overall sterile environment when preparing protoplast.

For transfection of plasmids to test editing efficiency, protoplasts were resuspended to a final concentration of 2×10⁵ cells/mi and, for transfection of plasmids for RNA extraction, protoplasts were resuspended to a final concentration of 5×10⁵ cells/ml. Transfection of protoplasts was performed by adding 20 μl of plasmid to 200 μl of protoplasts. Plasmid amounts are approximately the same within each experiment so that results are comparable. The plasmids and cells were mixed by gently tapping the tube 3-4 times. Then 220 μl of fresh and sterile PEG-CaCl₂ solution (PMID: 17585298) was added to the protoplast-plasmid mixture and mixed well by gently tapping tubes. The protoplasts with PEG were incubated at RT for 10 min, then 880 μl W5 solution (PMID: 17585298) was added and mixed with the protoplasts by inverting the tube 2-3 times to stop the transfection. Protoplasts were harvested by centrifuging tubes at 100 rcf for 2 min and resuspended in 1 ml of WI solution. They were then plated in 6-well plates pre-coated with 5% calf serum.

To harvest transfected protoplasts testing editing efficiency, protoplasts were either incubated at 23° C. for 48 hours (23° C. set) or incubated first at 23° C. for 12 hours, then moved to 37° C. for 2.5 hours, and finally, moved back to 23° C. for the remaining 33.5 hours (37° C. set). At the end of the incubations, the protoplasts were harvested by centrifugation at 100 rcf for 2-3 min. The resulting supernatant was moved to another tube and went through another centrifugation at 3000 rcf for 3 min to collect any residual protoplasts. Pellets from these two centrifugations were combined and flash frozen for further analysis.

To harvest transfected protoplasts for RNA extraction, protoplasts were incubated at room temperature (23° C.) for 36 hours. At the end of incubations, protoplasts were harvested by centrifugation at 100 rcf for 10 min. For RNA extraction, 6 wells of protoplasts transfected with the same plasmid were pooled.

Amplicon Sequencing

DNA of protoplast samples were extracted with Qiagen DNeasy plant mini kit. The amplicon was obtained using two rounds of PCR. Amplification primers for the first round of PCR were designed to have the 3′ sequence of the primer flanking a 200-300 bp fragment of the AtPDS3 gene around the area targeted by the guide RNA of interest. The 5′ part of the primer contains a sequence which will be bound by common sequencing primers (for reading paired-end reads, read 1 and read 2). The primers were designed so that the gRNA target sequence starts from within 100 bp of the beginning of read 1. The first round of PCR was done with the Thermo Phusion enzyme and half of all DNA extracted from a protoplast sample as template. After 25 cycles of amplification, the reaction was cleaned using 1× Ampure XP beads. The eluate was used as template for the second round of PCR using the Phusion enzyme and 12 cycles of amplification. The second round of PCR was designed so that indexes were added to each sample. The samples were then purified using 0.8× Ampure XP. Then amplicons were sent for next generation sequencing.

Amplicon Sequencing Result Analysis

Reads were first quality and adaptor trimmed with trim-galore and then mapped to the AtPDS3 genomic region by BWA aligner. Sorted and indexed bam files were used as input files for further analysis by the CrispRvariants R package. Each mutation pattern with corresponding read counts were exported by the CrispRvariants R package. After assessing all control samples, a criterion to classify reads as reads with a deletion was established: only reads with a >=3 bp deletion of the same pattern (deletion of the same size starting at the same location) with >=100 read counts from a sample are counted as reads with a deletion. This criterion is established due to the observation of 1 bp indels and occasionally 2 bp deletions with read numbers>100 in control samples. Also observed were larger deletions that happen at very low frequencies (much lower than 100 reads) in control samples. These observations indicate that occasional PCR inaccuracy and low-quality sequencing in a small fraction of reads can result in deletion patterns with corresponding read number ranges as stated above in control samples. By employing such stringent criteria, it is believed that the deletion signals counted are true signal indicating editing events.

RNA Extraction and QPCR

RNA was extracted with trizol (Ambion 15596018) and Direct-zol RNA miniprep kit (ZYMO R2052). cDNA was synthesized with iScript cDNA synthesis kit (BIO-RAD 1708891) and QPCR was performed with guide RNA specific primers with IQ SYBR Green Supermix (BIO-RAD 1708882).

### Results

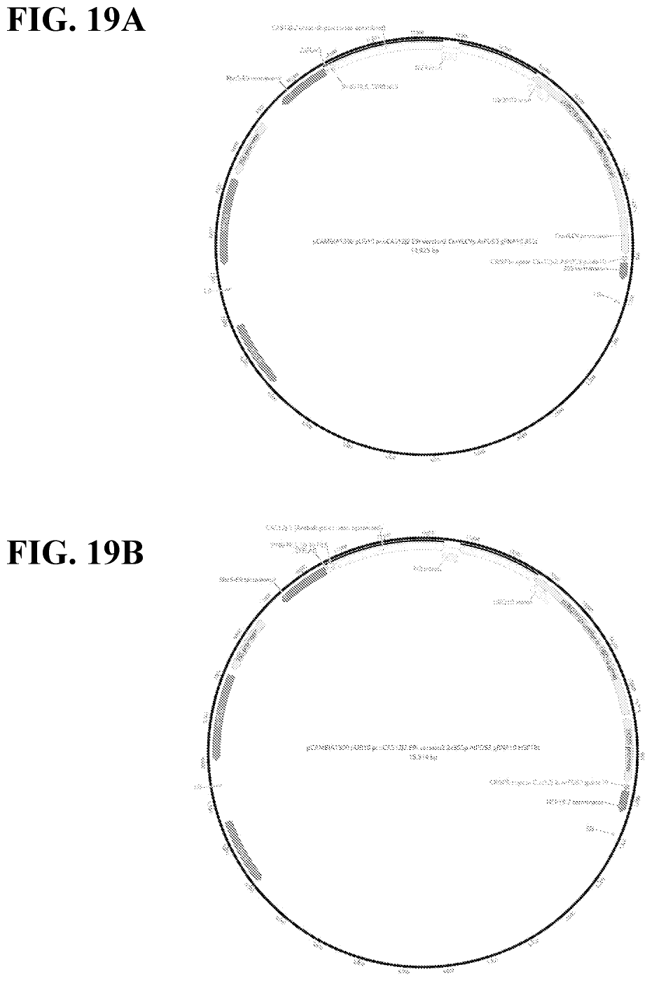

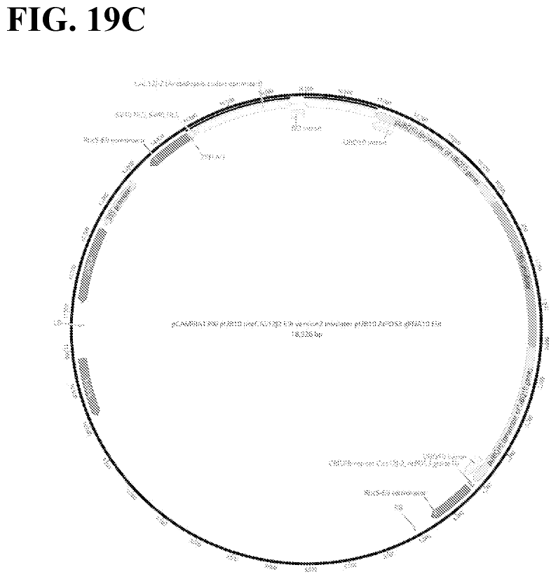

To test if Pol II promoters are able to drive CAS12J-2 guide RNA expression for editing, three combinations of constitutive Pol II promoter and terminator sets were selected: CmYLCV promoter+35S terminator, 2×35S promoter+HSP18.2 terminator and UBQ10 promoter+RbcS-E9 terminator. The constructed plasmids are shown in FIG. 19A-FIG. 19C. Since CAS12J-2 has intrinsic pre-crRNA processing activity (PMID: 32675376), it is likely not necessary to employ a secondary RNA processing mechanism to release the guide RNA from the Pol II transcript. Three gRNA configurations were tested with the Pol II promoter terminator combinations mentioned above: (1) a single CAS12J-2 repeat followed by AtPDS3 gRNA10; (2) a CAS12J-2 repeat followed by AtPDS3 gRNA10 with another CAS12J-2 repeat at the end; (3) a triple array of CAS12J-2 repeats followed by AtPDS3 gRNA10 with another CAS12J-2 repeat at the end (FIG. 20).

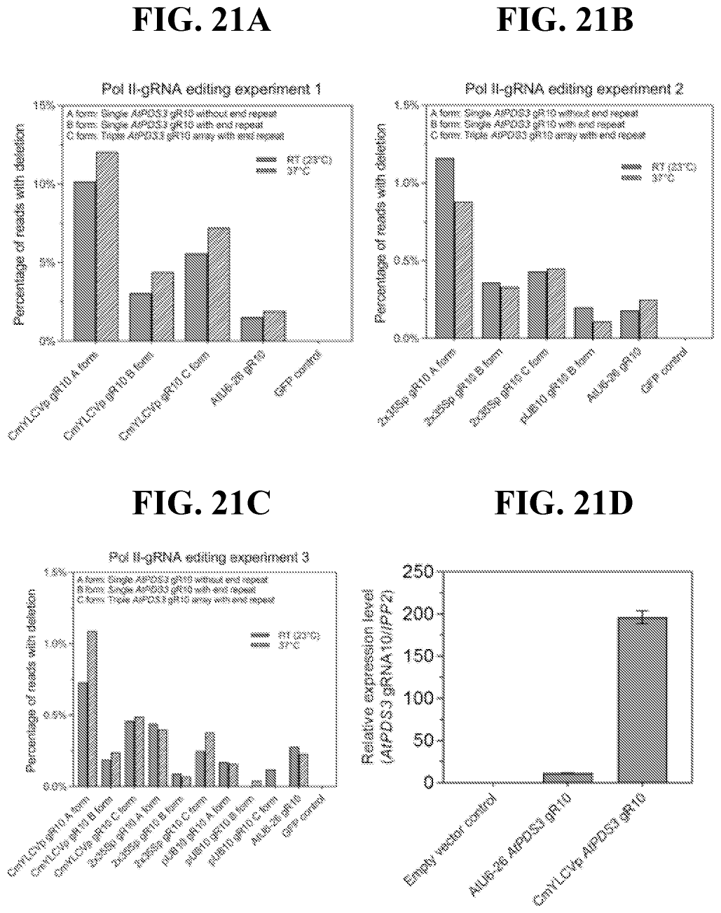

Three independent protoplast transfection experiments were performed to compare the editing efficiencies from different combinations with the original pCAMBIA1300 pUB10 pcoCAS12J2 E9t version2 AtU6-26 AtPDS3 gR10 plasmid transfection as control (FIG. 21A-FIG. 21C). Target gene editing was observed with all combinations of Pol II promoters and terminators, as well as gRNA configurations (FIG. 21A, FIG. 21B, FIG. 21C). Among the three combinations of Pol II promoters and terminators, the CmYLCV promoter with the 35S terminator led to the highest editing efficiency, while the UBQ10 promoter with the RbCS-E9 terminator led to the lowest editing efficiency (FIG. 21C). Out of the three different gRNA configurations, the single CAS12J-2 repeat followed by the AtPDS3 gRNA10 exhibited the highest editing efficiency, while the CAS12J-2 repeat followed by the AtPDS3 gRNA10 with another CAS12J-2 repeat at the end exhibited the lowest editing efficiency (FIG. 21A, FIG. 21B, FIG. 21C). When combining the CmYLCV promoter/35S terminator with the single CAS12J-2 repeat followed by the AtPDS3 gRNA10, the target gene editing efficiency was much higher than that of the AtU6-26 AtPDS3 gRNA10 cassette (FIG. 21A and FIG. 21C). The combination of 2×35S promoter/HSP18.2 terminator and a single CAS12J-2 repeat followed by the AtPDS3 gRNA10 also led to higher editing efficiency compared to the AtU6-26 AtPDS3 gRNA10 cassette (FIG. 21B and FIG. 21C). Consistent with the higher levels of editing observed, a higher level of AtPDS3 gRNA10 in protoplasts transfected with plasmid carrying the cassette with the CmYLCV promoter and single CAS12J-2 repeat followed by the AtPDS3 gRNA10 was also observed than in protoplasts transfected with the AtU6-26 AtPDS3 gRNA10 construct (FIG. 211)). This data suggests that boosting the levels of gRNAs can increase the efficiency of gene editing by CAS12J-2.

The fact that the single AtPDS3 gRNA10 without another CAS12J-2 repeat at the end exhibited the highest editing efficiency among the three gRNA configurations in FIG. 20 suggests that either CAS12J-2 processing is not efficient enough to fully release gRNA from Pol II transcript in planta, or more CAS12J-2 CRISPR repeats led to undesired complex RNA structures. The 20 bp spacer between the two CAS12J-2 CRISPR repeats could be too short to allow CAS12J-2 proteins binding simultaneously to both of the repeats for pre-crRNA processing without hindering each other's function. Also, adding in an efficient secondary gRNA processing machinery might be able to assist the release of free gRNA and further enhance editing efficiency. To examine this further, AtPDS3 gRNA10 with 30 bp spacer was used to test if longer spacer could assist the self-processing of pre-crRNA by CAS12J-2. Also, three secondary gRNA processing machineries were tested: (1) Ribozyme system (PMID 24373158); (2) Csy4 system (PMID 28522548); and (3) tRNA system (PMID 32483329).

When a single AtPDS3 gRNA10 without another CAS12J-2 repeat at the end was driven by CmYLCV promoter, no difference was observed between the editing efficiencies by the gRNA with 30 bp spacer and the gRNA with 20 bp spacer (FIG. 22B). This result suggests that the 30 bp spacer was not affecting the efficiency of target DNA editing. If the 30 bp spacer could enhance pre-crRNA processing by CAS12J, triple AtPDS3 gRNA10 with 30 bp spacer should yield more of the free gRNA compared to the triple AtPDS3 gRNA10 with 20 bp spacer and thus lead to higher editing efficiency. However, the triple AtPDS3 gRNA10 array with 30 bp spacer exhibited lower editing efficiency compared to the triple AtPDS3 gRNA10 array with 20 bp spacer (FIG. 22B), indicating that the longer 30 bp spacer was not promoting the processing of pre-crRNA by CAS12J-2.

To examine whether a secondary gRNA processing system is able to enhance editing efficiency, a ribozyme processing system was first used to assist the gRNA processing. The ribozyme processing system tested in this example employed a Hammerhead (HH) type ribozyme on the 5′ end of CAS12J-2 gRNA coding sequence and a hepatitis delta virus (HD) ribozyme on the 3′ end (FIG. 23A). A single CAS12J-2 AtPDS3 gRNA10 flanked by these ribozymes was cloned into the constructs with Pol II promoter gRNA cassettes, replacing the gRNA coding sequences without the processing machinery. Constructs with ribozymes led to significantly higher editing efficiency compared to the constructs without additional gRNA processing machinery, with all three promoter-terminator combinations tested (FIG. 23B). These results suggest that ribozymes were able to promote the processing of gRNA and the release of gRNA from the Pol II transcripts, leading to a higher editing efficiency.

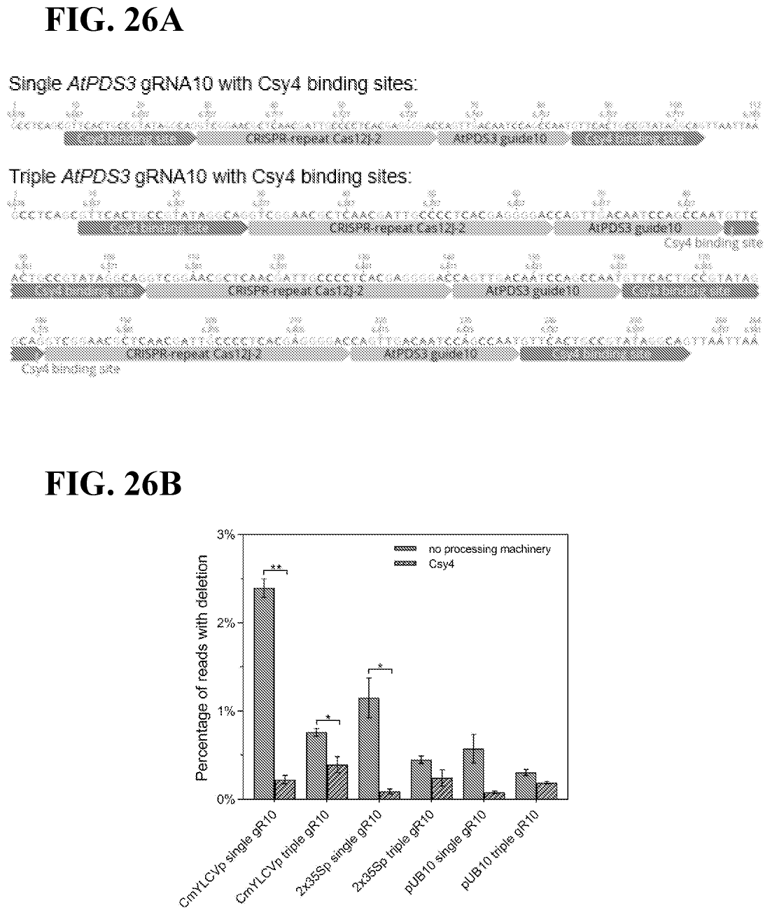

Csy4 gRNA processing system utilizes Csy-type ribonuclease 4 (Csy4) from Pseudomonas aeruginosa to bind the Csy4 recognition site and cleave the RNA at the 3′ end of the Csy4 recognition site (PMID 20829488, PMID 24770325). To examine if the Csy4 system could assist CAS12J-2 gRNA processing. Csy4 protein coding sequence was cloned at the N terminal of CAS12J-2 coding sequence separated by a 2A self-cleaving peptide (P2A) (See SEQ ID NO: 44), and the Csy4 binding sites were cloned to flank a single AtPDS3 gRNA10 or in the cased of the triple AtPDS3 gRNA10 array, flanking, as well as in between each gRNA (FIG. 26A). For all the three promoter-terminator combinations tested and for both single AtPDS3 gRNA10 or triple AtPDS3 gRNA10 array, either a decrease or non-significant difference in the editing efficiency was observed with the Csy4 processing system compared to the no secondary processing machinery control (FIG. 26B). Thus, these particular Csy4 constructions failed to enhance the editing efficiency by CAS12J-2.

As tRNA processing systems are also widely used for gRNA processing and multiplexing, it was also examined if the addition of tRNA processing system could increase the editing efficiency by CAS12J-2. Sequences encoding the full-length primary transcripts of methionine and isoleucine tRNAs were cloned to flank a single AtPDS3 gRNA10 (tRNAMet and tRNAIle) (FIG. 24). Also, a SacI restriction site (GAGCTC) and three nucleotides (TGA) were added to the 5′ side of the DNA sequences encoding the full-length primary transcripts of methionine and isoleucine tRNAs as in PMID 32483329. These longer tRNA sequences were named as long-tRNAMet and long-tRNAIle in this example. Long-tRNAMet and long-tRNAIle were also cloned to flank a single AtPDS3 gRNA10 (FIG. 24). CmYLCVp, 2×35Sp and pUB10 were also used to drive the expression of gRNA flanked by tRNAs. When the single AtPDS3 gRNA10 was flanked by all tRNA forms tested in this example, a significant decrease in editing efficiency was observed compared to the no processing machinery control (FIG. 25). This result suggests that the particular tRNA constructions used in this example were not able to promote processing of CAS12J-2 gRNA.

This example shows that Pol II promoters are able to effectively drive guide RNA expression for CAS12J-2 and cause target gene editing in vivo, without employing a separate guide RNA processing system such as ribozymes or Csy4. However, combining ribozyme gRNA processing machinery with Pol II promoters can further enhance the editing efficiency.

### Example 7: The Effect of Transgene Silencing on the Efficiency of CAS12J-2 Mediated Gene Editing

Plants have evolved to recognize genes from exogenous sources such as transgenes, viruses, and transposons, and are able to silence these exogenous genes. In this Example, CAS12J-2 transgenic plants were generated in Col-0 (WT) background and rdr6 mutant background and higher editing efficiencies were observed in transgenic plants in rdr6 mutant background. Thus, CAS12J-2 transgenes are also significantly affected by silencing mechanisms.

### Materials and Methods

Agrobacterium-Mediated Transformation and Selection of Transgenic T1 Plants were Performed as Described in Example 4.

The T1 plants in this example were generated by Agrobacterium-mediated transformation of pCAMBIA1300_pUB10_-pcoCAS12J2_E9t_version1_AtPDS3_gRNA10 and pCAMBIA1300_pUB10_pcoCAS12J2_E9t_version2_AtPDS3_gRNA10 plasmids in Col-0 (WT) and rdr6-15 mutant (PMID 15565108) background. Ten transgenic T1 plants for each plasmid in each background were randomly selected for amplicon sequencing after genotyping confirmation of the transgene and the genetic background. For transgenic T1 plants of pCAMBIA1300_pUB10_pcoCAS12J2_E9t_version2_AtPDS3_gRNA10 plasmid in rdr6-15 mutant background, only 9 transgenic plants were obtained after genotyping.

DNA Extraction and Amplicon Sequencing

To extract DNA from the transgenic plants, 2-3 cauline leaves were collected from each T1 plant. The cauline leaves from the same T1 plant were pooled together for DNA extraction.

Amplicon sequencing and amplicon sequencing result analysis were performed as described in Example 4.

### Results

Transgene silencing in plants is a prevalent phenomenon. While it is a well-evolved protection mechanism, transgene silencing poses many problems to research and agriculture applications. Transgene silencing occurs at multiple levels, including post transcriptional transgene silencing (PTGS), translational gene silencing and DNA methylation mediated transgene silencing. In Arabidopsis, RNA-dependent RNA polymerase 6 (RDR6) generates double stranded-RNA (dsRNA) using single-stranded RNA (ssRNA), such as the transcript from a transgene as template (PMID 10850496. PMID 10850495). The dsRNA products serve as substrate for the production of various kinds of siRNAs which trigger transgene silencing at multiple levels.

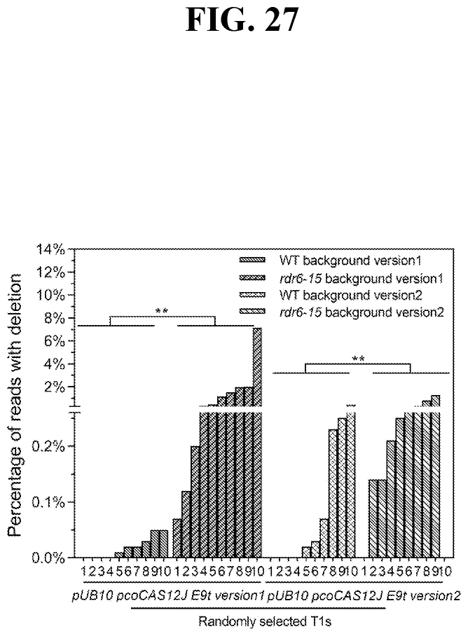

To evaluate if the CAS12J-2 transgene is also affected by transgene silencing, the editing efficiencies in CAS12J-2 transgenic plants were compared between the transgenic plant populations generated in Col-0 (WT) background and in the rdr6-15 mutant background. For transgenic plants generated from both the pCAMBIA1300 pUB10 pcoCAS12J2 E9t version1 AtPDS3 gRNA 10 plasmid and the pCAMBIA1300 pUB10 pcoCAS12J2 E9t version2 AtPDS3 gRNA 10 plasmid, significant increase in CAS12J-2 editing efficiency was detected in the population of T1 transgenic plants in the rdr6-1S mutant background compared to the WT background (FIG. 27). This result suggests that RDR6 mediated silencing mechanism negatively influenced the editing efficiency in CAS12J-2 transgenic plants.

The results of this example suggest that editing efficiency of CAS12J-2 transgenic plants is affected by transgene silencing. Thus, when high editing efficiency by CAS12J-2 is desired, strategies against transgene silencing may want to be considered. The rdr6 mutant is an exemplary and desirable genetic background to use which has minimal transgene silencing. In Arabidopsis, the rdr6 mutant is viable without many growth defects under lab conditions. Thus, use of the rdr6 mutant background may present a viable solution to transgene silencing.

What is claimed is:

1. A method for modifying a target nucleic acid in a plant cell, the method comprising:
a) providing a plant cell comprising a recombinant Cas12J polypeptide and a guide RNA;
b) cultivating the plant cell under conditions whereby the Cas12J polypeptide and guide RNA are present as a complex that targets the target nucleic acid to generate a modification in the target nucleic acid.

2. The method of claim 1, wherein the recombinant Cas12J polypeptide comprises an amino acid sequence having at least 80% amino acid identity to SEQ ID NO: 2.

3. The method of any one of claims 1-2, wherein the recombinant Cas12J polypeptide comprises a nuclear localization signal (NLS).

4. The method of claim 3, wherein the nuclear localization signal is an SV40-type NLS.

5. The method of any one of claims 1-4, wherein the recombinant Cas12J polypeptide and guide RNA are encoded from one or more recombinant nucleic acids in the plant cell.

6. The method of claim 5, wherein one of more of the recombinant nucleic acids comprise at least one intron.

7. The method of claim 5, wherein one of more of the recombinant nucleic acids comprise a promoter that is functional in plants.

8. The method of claim 7, wherein the promoter is a UBQ10 promoter.

9. The method of claim 8, wherein the UBQ10 promoter comprises a nucleic acid sequence that is at least 80% identical to SEQ ID NO: 23.

10. The method of any one of claims 5-9, wherein expression of the guide RNA is driven by an RNA Polymerase II promoter.

11. The method of claim 10, wherein the RNA Polymerase II promoter is a CmYLCV promoter or a 2×35S promoter.

12. The method of claim 11, wherein the promoter comprises a nucleic acid sequence that is at least 80% identical to SEQ ID NO: 29 or SEQ ID NO: 34.

13. The method of any one of claims 1-12, wherein the plant cell is cultivated at a temperature in the range of about 23° C. to about 37° C.

14. The method of any one of claims 1-12, wherein the plant cell is cultivated at a temperature in the range of about 20° C. to about 25° C.

15. The method of any one of claims 1-14, wherein the modification comprises a deletion of one or more nucleotides in the target nucleic acid.

16. The method of claim 15, wherein the deletion comprises deletion of 3-15 nucleotides in the target nucleic acid.

17. The method of claim 16, wherein the deletion comprises deletion of 9 nucleotides in the target nucleic acid.

18. The method of any one of claims 1-17, wherein the target nucleic acid sequence is located in a region of repressive chromatin.

19. The method of any one of claims 1-18, wherein the target nucleic acid sequence is located in a region of open chromatin.

20. The method of any one of claims 1-19, wherein the guide RNA is recombinantly fused to a ribozyme.

21. The method of any of claims 1-20, wherein the plant cell comprises a genetic background that exhibits reduced susceptibility to transgene silencing.

22. A recombinant vector comprising a nucleic acid sequence that includes a promoter that is functional in plants and that encodes a recombinant Cas12J polypeptide and a guide RNA.

23. A plant cell comprising a recombinant Cas12J polypeptide and a guide RNA, wherein the Cas12J polypeptide and guide RNA are capable of existing in a complex that targets a target nucleic acid to generate a modification in the target nucleic acid.

24. A plant comprising the plant cell of claim 23, wherein the plant comprises a modified nucleic acid.

25. A progeny plant of the plant of claim 24, wherein the progeny plant comprises a modified nucleic acid.
